(19) United States
(12) Patent Application Publication
(10) Pub. No.: US 2022/0089752 A1
(43) Pub. Date: Mar. 24, 2022

(54) COMPOSITIONS AND METHODS FOR MODULATING CELLULAR INTERNALIZATION

(71) Applicant: The Regents of the University of California, Oakland, CA (US)

(72) Inventors: Bin Liu, San Francisco, CA (US); Namkyung Lee, San Francisco, CA (US); Yang Su, San Francisco, CA (US); Scott Bidlingmaier, San Francisco, CA (US)

(21) Appl. No.: 17/422,591

(22) PCT Filed: Jan. 14, 2020
(86) PCT No.: PCT/US20/13433

§ 371 Date: Jul. 13, 2021

Related U.S. Application Data
(60) Provisional application No. 62/792,359, filed on Jan. 14, 2019.

(51) Int. Cl. C07K 16/28; A61K 47/68; A61P 35/00

(52) U.S. Cl. CPC C07K 16/2866; A61K 47/6849; A61K 47/6805; A61K 47/6817; A61K 47/6803; A61K 47/6809; A61K 2039/505; A61P 35/00; C07K 2317/56; C07K 2317/565; C07K 2317/73; C07K 2317/622; C07K 16/2803

(57) ABSTRACT

Provided herein are compositions and methods for modulating internalization properties of cell surface molecules, e.g., converting a non-internalizing cell surface antigen into an internalizing one, and vice versa. In some embodiments, provided are engineered antibodies each containing an antigen binding moiety specific for a guide-antigen and another antigen binding moiety specific for an effector antigen, wherein the internalization property of the engineered antibody or functional fragment thereof is determined by a relative surface density ratio of the guide antigen to the effector antigen. Also provided are recombinant cells, recombinant nucleic acids encoding such engineered antibodies, as well as pharmaceutical compositions containing same. The disclosure also provides methods useful for modulating cellular internalization in a cell or a subject, as well as methods for modulating cell-type selective signaling in a subject and/or for the treatment of diseases.

### CROSS-REFERENCE TO RELATED APPLICATIONS

This application claims the benefit of priority to U.S. Provisional Patent Application Ser. No. 62/792,359, filed on Jan. 14, 2019, which is herein expressly incorporated by reference in its entirety, including any drawings.

### STATEMENT REGARDING FEDERALLY SPONSORED R&D

This invention was made with government support under grant no. R01 CA118919, R01 CA129491 and R01 CA171315 awarded by the National Institutes of Health. The government has certain rights in the invention.

### INCORPORATION OF THE SEQUENCE LISTING

The present application is being filed along with a Sequence Listing in electronic format. The Sequence Listing is provided as a file entitled “Sequence Listing_048536-628001WO.txt”, created Dec. 23, 2019, which is approximately 124 KB in size. The information in the electronic format of the Sequence Listing is incorporated herein by reference in its entirety.

### FIELD

Aspects of the present application relate to the fields of cell biology and immunology. More particularly, provided herein are engineered antibodies that modulate and/or amplify cell type-specific internalization, e.g., converting a non-internalizing cell type-selective surface antigen into an internalizing one, and vice versa. The disclosure also provides compositions and methods useful for producing such engineered antibodies, as well as methods for the treatment of health disorders or diseases, such as diseases associated with cancer, including solid tumor and hematologic malignancy.

### BACKGROUND

Biopharmaceuticals or the use of pharmaceutical compositions comprising a therapeutic protein for the treatment of diseases, disorders, or health conditions is a core strategy for a number of pharmaceutical and biotechnology companies. For example, in cancer immunotherapy, the development of antibodies and antibody-drug conjugates (ADCs) that can target specific cancerous cells to prevent their proliferation and/or to kill the same, has emerged as a promising therapeutic approach to complement existing treatment strategies.

In particular, the high specificity of monoclonal antibodies is often exploited for targeted therapy development. Ideally, a potent cytotoxic agent attached to a cell-type specific antibody can route the cytotoxic agent to the target cell and preferentially accumulate in the target tissue. Another example of targeted therapeutics involves antibody-drug conjugates (ADCs) which have shown promising effectiveness in a number of clinical studies.

Although conceptually straightforward, target selection in therapeutic antibodies and ADCs is hindered by the findings that it is rare to find the so-called tumor specific antigens, and even rarer to find those that bear features desired for therapeutic targeting, i.e., uniformly expressed at high levels by cancer cells, and efficiently internalizing. In addition, translocation through the plasma membrane is a major limiting step for the cellular delivery of macromolecules. Therefore, the effectiveness of therapies relying on cellular internalization of a therapeutic depends on both the quality of their target on the surface of the target cells and the rate of cellular internalization of surface-bound therapeutic complexed with its target. In addition, internalizing therapeutic antibody is often desired to achieve efficient intracellular payload delivery and tumor killing, although the requirement is not absolute for certain drugs such as monomethyl auristatin E (MMAE) that can diffuse through cell membrane to cause a bystander effect. In some cases, in targeted therapy where intracellular payload delivery is required, many tumor antigens are highly expressed but poorly internalizing. In other cases, receptor internalization, which is a receptor-mediated endocytosis process that results in the movement of receptors from the plasma membrane to the inside of the cell, is also used to shut down signaling pathways, resulting in desensitization.

Thus, there is an ongoing need for new approaches and compositions for the treatment of diseases, disorders, or health conditions such as, e.g., inflammatory diseases, immune diseases, and cancers. In particular, a need exists in the art for more effective compositions and methods that treat diseases, disorders, or health conditions by improving internalization properties of therapeutic antibodies and ADCs.

### SUMMARY

This section provides a general summary of the disclosure and is not comprehensive of its full scope or all of its features.

The present disclosure relates to the compositions and methods for manipulation of cell-type selective antibody internalization by a guide-effector bispecific antibody design. In particular, provided herein are engineered antibodies that are capable of co-engaging a pair of antigens, termed “guide antigen” and “effector antigen,” that are expressed on the surface of the same cell. When being co-engaged by the engineered antibodies, the guide antigen can influence cell surface dynamics and/or signaling function of the effector antigen. In some particular designs, the effector antigen is an antigen associated with a target signaling pathway, and the guide antigen provides cell-type specificity to redirect and enhance the effector function to cells of interest. For example, a non-internalizing effector antigen can be converted to an internalizing effector antigen by using a guide-effector bispecific antibody design which is capable of binding (i) the non-internalizing effector antigen and (ii) an internalizing guide antigen. Similarly, an internalizing effector antigen can be converted to a non-internalizing effector antigen by using a guide-effector bispecific antibody design which is capable of binding (i) the internalizing effector antigen and (ii) a non-internalizing guide antigen, As described in greater detail below, modulation of internalization in some cases directly affects intracellular payload delivery and receptor signaling. Also provided are recombinant cells, recombinant nucleic acids encoding such engineered antibodies, as well as pharmaceutical compositions containing same. The disclosure also provides compositions and methods useful for modulating cellular internalization in a cell or a subject by using such engineered antibodies, as well as methods for modulating cell-type selective signaling in a subject and/or for the treatment of health disorders and diseases, such as diseases associated with cancer, including solid tumor and hematologic malignancy.

In one aspect, some embodiments of the present disclosure relate to an engineered antibody or functional fragment thereof including: a) a first antigen binding moiety capable of binding to a cell-surface guide antigen having a first rate of cellular internalization; and b) a second antigen binding moiety capable of binding to a cell-surface effector antigen having a second rate of cellular internalization, wherein the internalization property of the engineered antibody or functional fragment thereof is determined by a relative surface density ratio of the guide antigen to the effector antigen, and wherein one of the two cellular internalization rates is at least 50%, at least 70%, at least 80%, or at least 90% greater than the other rate.

Implementations of embodiments of the engineered antibody of the present disclosure can include one or more of the following features. In some embodiments, the cell-surface guide antigen is an internalizing cell surface antigen. In some embodiments, the cell-surface effector antigen is a non-internalizing cell surface antigen. In some embodiments, the relative surface density ratio of the guide antigen to the effector antigen is greater than a threshold value. In some embodiments, the relative surface density ratio of the guide antigen to the effector antigen is below a threshold value. In some embodiments, the threshold value is about 1:1, about 1:2, about 1:3, about 1:4, about 1:5, about 1:10, about 1:20, or about 1:30. In some embodiments, the first antigen binding moiety and the second antigen binding moiety are independently selected from the group consisting of an antigen-binding fragment (Fab), a single-chain variable fragment (scFv), a full-length immunoglobulin, a nanobody, a single domain antibody (sdAb), a variable new antigen receptor (VNAR) domain, and a VHH domain, a multispecific antibody, a diabody, or a functional fragment thereof. In some embodiments, the guide antigen and the effector antigen are independently selected from the group consisting of activated leukocyte cell adhesion molecule (ALCAM), neural cell adhesion molecule (NCAM), calcium-activated chloride channel 2 (CaCC), carbonic anhydrase IX, carcinoembroyonic antigen (CEA), cathepsin G, CD19, CD20, CD22, CD30, CD33, CD38, CD44, CD44v6, CD46, CD52, CD71, CD73, CD272, CD276, B-cell maturation antigen (BCMA), epithelial cell adhesion molecule (EpCAM), ephrin type-A receptor 2 (EphA2), ephrin type-A receptor 3 (EphA3), ephrin type-A receptor 4 (EphA4), ephrin B2, receptor tyrosine kinase like orphan receptor 1 (ROR1), folate receptor, FLT3 (CD135), KIT (CD117), CD213A2, IL-1Ra, PRSS21, VEGFR2, CD24, PDGFR-beta, SSEA-4, epidermal growth factor receptor (EGFR), Erb-B2 receptor tyrosine kinase 2 (ErbB2), Erb-B2 receptor tyrosine kinase 3 (ErbB3), Erb-B2 receptor tyrosine kinase 4 (ErbB4), folate binding proteins (folate receptors), ganglioside, gangliosides, gp100, gpA33, immature laminin receptor, intercellular adhesion molecule 1 (ICAM-1), Lewis-Y, mesothelin, prostate stem cell antigen (PSCA), mucin 16 (MUC16 or CA-125), mucin 1 cell-surface associated (MUC1), mucin 2 oligomeric mucus gel-forming (MUC2), mucins, prostate membrane specific antigen (PSMA), TEM1/CD248, TEM7R, CLDN6, thyroid stimulating hormone receptor (TSHR), GPRC5D, CD97, CD179a, anaplastic lymphoma kinase (ALK or CD246), immunoglobulin lambda like polypeptide 1 (IGLL1), P-selectin, c-Met, fibroblast growth factor receptors (FGFRs), insulin-like growth factor 1 receptor (IGF-1R), tumor-associated calcium signal transducer 2 (Trop-2), and tumor associated glycoprotein 72 (TAG-72).

In some embodiments, the guide antigen is a cancer-associated antigen selected from the group consisting of CD19, CD22, HER2 (ErbB2/neu), mesothelin, PSCA, CD123, CD30, CD71, CD171, CS-1, CLECL1, CD33, EGFRvIII, GD2, GD3, BCMA, PSMA, receptor tyrosine kinase like orphan receptor 1 (ROR1), folate receptor, FLT3 (CD135), TAG72, CD38, CD44v6, CD46, CEA, EpCAM, CD272, B7H3 (CD276), KIT (CD117), CD213A2, IL-1Ra, PRSS21, VEGFR2, CD24, PDGFR-beta, SSEA-4, CD20, MUC1, MUC16, EGFR, ErbB2, ErbB3, ErbB4, NCAM, prostatic acid phosphatase (PAP), ephrin B2, fibroblast activation protein (FAP), EphA2, c-Met, fibroblast growth factor receptors (FGFRs), insulin-like growth factor 1 receptor (IGF-1R), GM3, TEM1/CD248, TEM7R, CLDN6, thyroid stimulating hormone receptor (TSHR), GPRC5D, CD97, CD179a, anaplastic lymphoma kinase (ALK or CD246), and immunoglobulin lambda like polypeptide 1 (IGLL1).

In some embodiments, the effector antigen is selected from the group consisting of ALCAM, EpCAM, Folate binding proteins, PSMA, PSCA, mesothelin, CD19, CD20, CD22, CD30, CD33, CD38, CD44, CD46, ICAM-1, CD55, CD59, CD70, CD71, CD73, CD97, BCMA, CD272, CD276, MUC1, MUC16, NCAM, CD24, EphA2, EphA3, EphA4, Ephrin B2, CEA, c-Met, FGFRs, IGF-1R, VEGFRs, PDGFRs, Trop-2, TAG-72, P-selectin, EGFR, ErbB2, ErbB3, and ErbB4.

In some embodiments, the antibody or functional fragment thereof is conjugated or covalently bound to at least one moiety-of-interest (MOI) selected from the group consisting of therapeutic moieties, diagnostic agents, and moieties that improve pharmacokinetics. In some embodiments, the at least one MOI is selected from the group consisting of an anticancer agent, an anti-autoimmune disease agent, an anti-inflammatory agent, an anti-bacterial agent, an antimicrobial agent, an antibiotic, an anti-infectious disease agent, and an antiviral agent. In some embodiments, the at least one MOI is selected from the group consisting of cytotoxic anti-cancer agents, DNA chelators, microtubule inhibitors, topoisomerase inhibitors, translation initiation inhibitors, ribosome inactivating molecules, nuclear transport inhibitors, RNA splicing inhibitors, RNA polymerase inhibitors, and DNA polymerase inhibitors.

In some embodiments, the cytotoxic anti-cancer agent is selected from the group consisting of auristatins, dolastatins, tubulysins, maytansinoids, taxanes, vinca alkaloids, amatoxins, anthracyclines, calicheamycins, camptothecins, irinotecan, SN-38, combretastatins, duocarmycins, enediynes, epothilones, ethylenimines, mytomycins, pyrrolobenzodiazepines (PBDs), and calicheamicin.

In some embodiments, the at least one moiety-of-interest (MOI) is conjugated or covalently bound to a constant region of the engineered antibody or functional fragment thereof. In some embodiments, the at least one moiety-of-interest (MOI) is conjugated or covalently bound to a heavy chain constant (e.g., CH1, CH2, or CH3) region of the antibody or functional fragment thereof. In some embodiments, the at least one moiety-of-interest (MOI) is conjugated or covalently bound to a heavy chain constant (CH1) region of the antibody or functional fragment thereof. In some embodiments, the at least one moiety-of-interest (MOI) is conjugated or covalently bound to a light chain constant (CL) region of the antibody or functional fragment thereof. In some embodiments, the mean number of MOIs per antibody (e.g., mean drug to antibody ratio, DAR) ranges from 1 to 20. In some embodiments, the mean DAR is about 1 to 5 about, about 2 to about 6, about 3 to about 7, about 3 to about 8, about 4 to about 9, about 5 to about 10, about 10 to about 15, about 15 to about 20, or about 10 to about 20.

In some embodiments, the engineered antibody or functional fragment disclosed herein includes a first antigen binding moiety capable of binding to an EphA2 expressed on the surface of a cell; and a second antigen binding moiety capable of binding to an ALCAM expressed on the surface of the same cell. In some embodiments, the surface density ratio of EphA2 to ALCAM is greater than a threshold value of about 1:5. In some embodiments, the engineered antibody or functional fragment thereof as described herein includes an amino acid sequence having at least 80% sequence identity to any one of the amino acid sequences identified in Table 4. In some embodiments, the first antigen binding moiety includes a heavy chain variable (VH) region having at least 80% sequence identity to a VH sequence identified in Table 4. In some embodiments, the first antigen binding moiety includes a VH region having at least 80% sequence identity to SEQ ID NO: 81 or SEQ ID NO: 96. In some embodiments, the VH region of the first antigen binding moiety includes three complementary determining regions (HCDRs) as identified in the Sequence Listing. In some embodiments, the VH region of the first antigen binding moiety includes HCDR1, HCDR2, and HCDR3 including SEQ ID NO: 104, SEQ ID NO: 105, and SEQ ID NO: 106, respectively; or SEQ ID NO: 104, SEQ ID NO: 105, and SEQ ID NO: 110, respectively. In some embodiments, the first antigen binding moiety includes a light chain variable (VL) region having at least 80% sequence identity to a VH sequence identified in Table 4. In some embodiments, the first antigen binding moiety includes a VL region having at least 80% sequence identity to SEQ ID NO: 82 or SEQ ID NO: 97. In some embodiments, the VL region of the first antigen binding moiety includes three LCDRs as identified in the Sequence Listing. In some embodiments, the VL region of the first antigen binding moiety includes LCDR1, LCDR2, and LCDR3 including SEQ ID NO: 107, SEQ ID NO: 108, and SEQ ID NO: 109, respectively.

In some embodiments, the second antigen binding moiety includes a VH region having at least 80% sequence identity to a VH sequence identified in Table 4. In some embodiments, the second antigen binding moiety includes a VH region having at least 80% sequence identity to SEQ ID NO: 73 or SEQ ID NO: 75. In some embodiments, the VH region of the second antigen binding moiety includes three HCDRs as identified in the Sequence Listing. In some embodiments, the VH region of the second antigen binding moiety includes HCDR1, HCDR2, and HCDR3 including SEQ ID NO: 98, SEQ ID NO: 99, and SEQ ID NO: 100, respectively. In some embodiments, the second antigen binding moiety includes a VL region having at least 80% sequence identity to a VH sequence identified in Table 4. In some embodiments, the second antigen binding moiety includes a VL region having at least 80% sequence identity to SEC ID NO: 74 or SEQ ID NO: 76. In some embodiments, the VL region of the second antigen binding moiety includes three LCDRs as identified in the Sequence Listing. In some embodiments, the VL region of the second antigen binding moiety includes LCDR1, LCDR2, and LCDR3 including SEQ ID NO: 101, SEQ ID NO: 102, and SEQ ID NO: 103, respectively.

In one aspect, some embodiments of the present disclosure relate to a recombinant nucleic acid molecule including a nucleic acid sequence that encodes an engineered antibody or functional fragment thereof as disclosed herein. In some embodiments, the recombinant nucleic acid molecule is operably linked to a heterologous nucleic acid sequence. In some embodiments, the recombinant nucleic acid molecule is further defined as an expression cassette or a vector.

In one aspect, some embodiments of the present disclosure relate to a recombinant cell including (a) an engineered antibody or functional fragment thereof as disclosed herein; and/o (b) a nucleic acid molecule as disclosed herein. In some embodiments, the recombinant cell is a prokaryotic cell or a eukaryotic cell. In a related aspect, some embodiments of the present disclosure relate to a cell culture including at least one recombinant cell as disclosed herein and a culture medium.

In one aspect, some embodiments of the present disclosure relate to a pharmaceutical composition including one or more of the following: (a) an engineered antibody or functional fragment thereof as disclosed herein; (b) a nucleic acid molecule as disclosed herein; and (c) a recombinant cell as disclosed herein, and a pharmaceutically acceptable carrier.

In another aspect, some embodiments of the present disclosure relate to a method for modulating cellular internalization, including administering to a cell one or more of the following: (a) an engineered antibody or functional fragment thereof as disclosed herein; (b) a nucleic acid molecule as disclosed herein; and (c) a pharmaceutical composition as disclosed herein.

In another aspect, some embodiments of the present disclosure relate to a method for modulating cellular internalization, the method includes administering to a cell an engineered antibody or functional fragment thereof including: (a) a first antigen binding moiety capable of binding to a cell-surface guide antigen having a first rate of cellular internalization; and (b) a second antigen binding moiety capable of binding to a cell-surface effector antigen having a second rate of cellular internalization, wherein the internalization property of the engineered antibody or functional fragment thereof is determined by a relative surface density ratio of the guide antigen to the effector antigen, and wherein one of the two cellular internalization rates is at least 50%, at least 70%, at least 80%, or at least 90% greater than the other rate.

In yet another aspect, some embodiments of the present disclosure relate to a method for modulating cell-type selective signaling in a subject, the method includes administering to a cell an engineered antibody or functional fragment thereof including: (a) a first antigen binding moiety capable of binding to a cell-surface guide antigen, wherein the guide antigen is expressed in the subject in a cell-type selective manner and has a first rate of cellular internalization; and (b) a second antigen binding moiety capable of binding to a cell-surface effector antigen having a second rate of cellular internalization, wherein the internalization property of the engineered antibody or functional fragment thereof is determined by a relative surface density ratio of the guide antigen to the effector antigen, and wherein one of the two cellular internalization rates is at least 50%, at least 70%, at least 80%, or at least 90% greater than the other rate.

In yet another aspect, some embodiments of the present disclosure relate to a method for treating a health condition or diseases in a subject in need thereof, the method includes administering to the subject a therapeutically effective amount of an engineered antibody or functional fragment thereof as disclosed herein. In some embodiments, engineered antibody or functional fragment thereof includes: (a) a first antigen binding moiety capable of binding to a cell-surface guide antigen having a first rate of cellular internalization; and (b) a second antigen binding moiety capable of binding to a cell-surface effector antigen having a second rate of cellular internalization, wherein the internalization property of the engineered antibody or functional fragment thereof is determined by a relative surface density ratio of the guide antigen to the effector antigen; and wherein one of the two cellular internalization rates is at least 50%, at least 70%, at least 80%, or at least 90% greater than the other rate. In some embodiments, the health condition or disease is a cancer.

In yet another aspect, some embodiments of the present disclosure relate to a method for killing a cancer cell, the method includes administering to said cell an engineered antibody or functional fragment thereof as disclosed herein. In some embodiments, the engineered antibody or functional fragment thereof includes: (a) a first antigen binding moiety capable of binding to a cell-surface guide antigen having a first rate of cellular internalization; and (b) a second antigen binding moiety capable of binding to a cell-surface effector antigen having a second rate of cellular internalization.

In yet another aspect, some embodiments of the present disclosure relate to a method for killing a tumor cell, the method includes administering to said tumor cell an engineered antibody or functional fragment thereof as disclosed herein. In some embodiments of the disclosed methods, the engineered antibody or functional fragment thereof includes a first antigen binding moiety capable of binding to an ephrin receptor A2 (EphA2) expressed on the surface of said tumor cell; and a second antigen binding moiety capable of binding to an ALCAM expressed on the surface of the same tumor cell. In some embodiments, the surface density ratio of EphA2 to ALCAM is greater than a threshold value of about 1:5.

In some embodiments of the disclosed methods, the engineered antibody or functional fragment thereof includes an amino acid sequence having at least 80% sequence identity to any one of the amino acid sequences identified in Table 4. In some embodiments, the first antigen binding moiety includes a VH region having at least 80% sequence identity to a VH sequence identified in Table 4. In some embodiments, the first antigen binding moiety includes a VH region having at least 80% sequence identity to SEQ ID NO: 81 or SEQ ID NO: 96. In some embodiments, the VH region of the first antigen binding moiety includes three HCDRs as identified in the Sequence Listing. In some embodiments, the VH region of the first antigen binding moiety includes HCDR1, HCDR2, and HCDR3 including: (a) SEQ ID NO: 104, SEQ ID NO: 105, and SEQ ID NO: 106, respectively; or (b) SEQ ID NO: 104, SEQ ID NO: 105, and SEQ ID NO: 110, respectively. In some embodiments, the first antigen binding moiety includes a VL region having at least 80% sequence identity to a VL sequence identified in Table 4. In some embodiments, the first antigen binding moiety includes a VL region having at least 80% sequence identity to SEQ ID NO: 82 or SEQ ID NO: 97. In some embodiments, the VL region of the first antigen binding moiety includes three LCDRs as identified in the Sequence Listing. In some embodiments, the VL region of the first antigen binding moiety includes LCDR1, LCDR2, and LCDR3 including SEQ ID NO: 107, SEQ ID NO: 108, and SEQ ID NO: 109, respectively.

In some embodiments of the disclosed methods, the second antigen binding moiety includes a VH region having at least 80% sequence identity to a VH sequence identified in Table 4. In some embodiments, the second antigen binding moiety includes a VH region having at least 80% sequence identity to SEQ ID NO: 73 or SEQ ID NO: 75. In some embodiments, the VH region of the second antigen binding moiety includes three HCDRs as identified in the Sequence Listing. In some embodiments, the VH region of the second antigen binding moiety includes HCDR1, HCDR2, and HCDR3 including SEQ ID NO: 98, SEQ ID NO: 99, and SEQ ID NO: 100, respectively. In some embodiments, the second antigen binding moiety includes a VL region having at least 80% sequence identity to a VL sequence identified in Table 4. In some embodiments, the second antigen binding moiety includes a VL region having at least 80% sequence identity to SEC ID NO: 74 or SEQ ID NO: 76. In some embodiments, the VL region of the second antigen binding moiety includes three LCDRs as identified in the Sequence Listing. In some embodiments, the VL region of the second antigen binding moiety includes LCDR1, LCDR2, and LCDR3 including SEQ ID NO: 101, SEQ ID NO: 102, and SEQ ID NO: 103, respectively.

In some embodiments, the cancer is a pancreatic cancer, a colon cancer, an ovarian cancer, a prostate cancer, a lung cancer, mesothelioma, a breast cancer, an urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the cell-surface guide antigen is an internalizing cell surface antigen. In some embodiments, the cell-surface effector antigen is a non-internalizing cell surface antigen. In some embodiments, the relative surface density ratio of the guide antigen to the effector antigen is greater than a threshold value. In some embodiments, the relative surface density ratio of the guide antigen to the effector antigen is below a threshold value. In some embodiments, the threshold value is about 1:1, about 1:2, about 1:3, about 1:4, about 1:5, about 1:10, about 1:20, or about 1:30. In some embodiments, the methods as disclosed herein further include modulating cell surface density of the guide antigen and/or cell surface density of the effector antigen. In some embodiments, the internalization property of the engineered antibody as disclosed herein is converted from internalizing to non-internalizing. In some other embodiments, the internalization property of the engineered antibody disclosed herein is converted from non-internalizing to internalizing. In some embodiments, the expression of the guide antigen and/or the effector antigen is cell-type selective.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1G summarize results from experiments performed to illustrate that a bispecific antibody based on the guide-effector design in accordance to some non-limiting embodiments of the disclosure can profoundly impact internalization dynamics of cell surface antigen. FIG. 1A is an illustration of the tetravalent ALCAMxEphA2 bsIgG. The IgG backbone is based on the non-internalizing anti-ALCAM antibody 3F1. The internalizing anti-EphA2 scFv is fused to the end of light chain C-terminus. FIG. 1B shows confocal microscopy study of antibody internalization. HEK293 or HEK293-EphA2 #2 cells were incubated with indicated IgG or bsIgG (100 nM) at 37° C. for 2 hours. Antibodies (red) were detected using Alexa® 647-labeled anti-human IgG secondary antibody, and cell images were analyzed using a digital laser confocal microscope. Scale bar: 20 μm. FIG. 1C depicts kinetics of ALCAM cell-surface removal by the bispecific antibody. HEK293-EphA2 #2 cells were incubated with indicated IgG or bsIgG for 1, 4, and 24 hours and surface ALCAM levels determined by FACS. The non-internalizing ALCAM is removed from cell surface by the bispecific (3F1/RYR) but not monoclonal antibodies. FIG. 1D depicts correlation between surface antigen (ALCAM) removal efficiency and EphA2/ALCAM (E/A) expression ratio. HEK293 cell models with varying EphA2/ALCAM ratios were incubated with 3F1, 3F1/RYR, and C10/RYR (all at 100 nM), and antigens remaining on the cell surface were determined by anti-ALCAM antibodies that bind to a different epitope than 3F1. Pearson's correlation coefficient (r) was calculated (0.3266, −0.7550, and −0.1896 for 3F1, 3F1/RYR, and C10/RYR, respectively) and trend-lines were depicted according to linear regression analysis. Data represent mean±SD (duplicate). FIG. 1E is an illustration depicting bispecific-induced ALCAM internalization when the guide to effector ratio >threshold. CM: cell membrane. FIG. 1F shows significant retardation of EphA2 internalization by the bispecific 3F1/RYR when guide to effector ratio falls below a threshold value. HEK293 cells that possess a low EphA2/ALCAM ratio (<0.2) were incubated with indicated antibodies (100 nM), and surface EphA2 levels were measured by FACS. P values were determined using two-tailed Student's t-test. *P<0.05, and ***P<0.001. FIG. 1G is an illustration of the phenomenon shown in FIG. 1F where EphA2 internalization is retarded (e.g., reduced) when the EphA2 to ALCAM (E/A) ratio falls below a threshold.

FIGS. 2A-2F summarize results from experiments performed to demonstrate that a bispecific antibody (3F1/RYR) based on the guide-effector design in accordance to some non-limiting embodiments of the disclosure effectively removes a non-internalizing antigen (ALCAM) from pancreatic cancer cell surface. FIG. 2A: ALCAM cell-surface level post antibody treatment. Pancreatic cancer cell lines L3.6pl (left bars on X-axis), Capan-1 (middle bars), and Panc-1 (right bars) were incubated with 3F1, 3F1/RYR, C10/RYR, or a mixture of 3F1 and C10/RYR. Following wash post treatment, cell-surface ALCAM level was determined using an Alexa® 647-labeled IgG that bind to a different epitope on ALCAM than 3F1. MFI values were normalized against cells without antibody treatment. **P<0.01, and ***P<0.001. Duplicates. FIG. 2B: Confocal microscopy study of cell-type selective internalization mediated by the bispecific antibody. L3.6pl (E/A ratio >0.2) and Panc-1 (E/A ratio <0.2) cells were incubated with 3F1, 3F1/RYR, or C10/RYR, and internalizing antibodies were stained with FITC-labeled anti-human IgG. Scale bar: 20 μm. FIG. 2C: Co-localization of antibodies and macropinocytotic vesicles. L3.6pl cells were incubated with 3F1, 3F1/RYR, or C10/RYR at 100 nM and ND70-TR (TR-Dextran, red) for 2 hours. Antibodies were detected by FITC-labeled anti-human IgG (green). Nuclei were labeled with Hoechst 33342 (blue). Scale bar: 10 μm. FIG. 2D: Lysosomal trafficking post internalization. L3.6pl cells were incubated with indicated antibodies (100 nM) for 2 hours. Internalized antibodies (green) and nuclei (blue) were stained as described in C), and lysosomes were detected using rabbit anti-LAMP1 primary IgG, followed by Alexa® 647-labeled anti-rabbit IgG (red). Scale bar: 10 μm. FIG. 2E: Retarded EphA2 internalization on Panc-1 cell when targeted by the bispecific antibody. **P<0.01, and ***P<0.001. Duplicates. FIG. 2F: A time course of EphA2 removal from Panc-1 cell surface at 0.5, 1, and 4 hours post antibody treatment.

FIGS. 3A-3E summarize results from experiments performed to demonstrate that the removal of bispecific-induced cell-surface ALCAM in accordance with some non-limiting embodiments of the disclosure has an anti-clonogenic effect against pancreatic tumor-spheres. FIG. 3A: Significant ALCAM upregulation on L3.6pl sphere cells compared to non-sphere tumor cells. Adherent- or sphere-cultured L3.6pl cells were separated into single cells and antigen expression was measured using 3F1 or RYR IgG, followed by Alexa® 647-labeled anti-human IgG. FIG. 3B: ALCAM removal from the surface of sphere-forming cells by 3F1/RYR. Single cell population of L3.6pl (200 cells/well) was incubated with indicated antibodies (100 nM) for 2 weeks in an ultra-low attachment well plate. Cell-surface levels of ALCAM post antibody treatment were determined by FACS. MFI values were normalized against control (no antibody treatment). **P<0.01. Duplicates. FIG. 3C: Antibody internalization into L3.6pl spheres. Tumor spheres incubated with indicated antibodies were collected by centrifugation, fixed, and permeabilized for confocal microscopy analysis. Antibodies and nuclei were stained with Alexa® 647-labeled anti-human IgG (red) and Hoechst 33342 (cyan), respectively. Scale bar: 10 μm. Intracellular antibody fluorescence intensity was quantified by Image J and shown in the right panel. ***P<0.001. FIG. 3D: Inhibition of L3.6pl tumor sphere formation by 3F1/RYR—reduction in number. Tumor sphere numbers (>100 μm) were counted 14 days post antibody treatment (left) with representative well images shown (right). Error bars represent SD of a duplicate. *P<0.05. FIG. 3E: Inhibition of L3.6pl tumor sphere formation by 3F1/RYR-reduction in size. **P<0.01. Duplicates. Scale bar: 100 μm.

FIGS. 4A-4E illustrates in vitro potency and selectivity of exemplary antibody-drug conjugates (ADCs) with site-specific conjugation on tumor cell lines with varying EphA2/ALCAM ratio in accordance with some non-limiting embodiments of the disclosure. Cytotoxicity of indicated ADCs or a mixture was studied on L3.6pl (FIG. 4A) and Capan-1 (FIG. 4B) cell lines with relatively high EphA2/ALCAM ratios, and Panc-1 (FIG. 4C) cell line that has a low EphA2/ALCAM ratio. MIA PaCa2 (FIG. 4D) and C4-2B (FIG. 4E) cell lines were utilized as an ALCAM-low/negative and EphA2-low/negative cancer cell model, respectively. Cell viability (%) was normalized against a control group without ADC treatment.

FIGS. 5A-5B illustrate the anti-tumor efficacy of an exemplary bispecific 3F1/RYR antibody-drug conjugate (ADC) in a pancreatic cancer xenograft model in accordance with some non-limiting embodiments of the disclosure. FIG. 5A: Effect on tumor growth. Mice were inoculated subcutaneously with 1×10⁶ Capan-1 cells and randomly divided into 4 groups (6 mice/group) with similar average tumor size. Vehicle (PBS) or ADCs (3 mg/kg) were intravenously injected at indicated time points (arrow head). The mean tumor volumes±SEM (mm³) was plotted. FIG. 5B: Body weight was monitored and plotted to assess toxicity of ADC treatment. No significant body weight loss (e.g., >15%) was seen for any of the groups studied.

FIGS. 6A-6D graphically illustrate the selection and characterization of anti-ALCAM scFvs from phage display library. FIG. 6A: Enrichment of ALCAM-binding phage through three rounds of selection. Recombinant Fc-fusion of ALCAM-V domain was immobilized on magnetic beads and utilized for scFv phage display library selection. Enrichment was calculated by dividing phage output titer by input phage titer (y-axis on the left). Binding activity of polyclonal phages amplified from each round output was described as folds against the binding of the unselected phage library (y-axis on the right). FIG. 6B: After three rounds of selection, FACS was performed to screen out monoclonal phage binding to the ALCAM^{high} DU145 cell line. FIG. 6C: Apparent K_{D} of 3F1 IgG on live ALCAM-expressing cells. DU145 cells were incubated with varying concentrations of 3F1 IgG at 4° C. overnight and analyzed by FACS using Alexa® 647-conjugated anti-human IgG. K_{D} value was estimated by curve fitting using GraphPad Prism (GraphPad Software). FIG. 6D: Confocal microscopy study of cellular localization of anti-ALCAM 3F1 IgG. Tumor cell lines were seeded in chamber-well slide and incubated with 3F1 IgG at 37° C. for 2 hours. Antibodies were stained with Alexa® 647-conjugated anti-human antibody (red). Nuclei were stained with Hoechst dye (cyan). Scale bar: m. ALCAM expression measured using 3F1 IgG was shown below microscopic images (lower panel).

FIGS. 7A-7B graphically illustrate the characterization of exemplary anti-ALCAMxEphA2 bispecific in accordance with some embodiments of the present disclosure. FIG. 7A: Reducing SDS-PAGE analysis of monoclonal (3F1 and C10) and bispecific (3F1/RYR and C10/RYR) antibodies. 3F1 or C10 IgG is composed of heavy (˜50 kDa) and light (˜25 kDa) chains. 3F1/RYR or C10/RYR bsIgG is composed of two similar-sized bands (˜50 kDa), a heavy chain and a light chain fused with a scFv. FIG. 7A: FACS analysis of binding specificity. Bispecific and monoclonal antibodies were incubated with the HEK293-EphA2 #2 cell line that stably expresses EphA2 and the parental HEK293 (as a specificity control), and analyzed by FACS.

FIGS. 8A-8C graphically illustrate the removal of a surface antigen in accordance with some embodiments of the present disclosure. FIG. 8A: Inefficient surface ALCAM removal on HEK293 cells that lack expression of the guide antigen EphA2. HEK293 cells were incubated with indicated IgG or bsIgG at 37° C. for 1, 4, and 24 hours, washed and analyzed by FACS to determine cell-surface ALCAM level post antibody treatment. FIG. 8B: EphA2 cell-surface removal in pancreatic cancer cell lines with varying EphA2 to ALCAM ratio. The anti-ALCAM 3F1 IgG did not reduce surface EphA2 as expected, but the 3F1/RYR and the control C10/RYR that binds to EphA2 removed EphA2 efficiently from the cell surface. The ability of the bispecific 3F1/RYR to remove surface ALCAM is affected by the ratio of EphA2 to ALCAM (guide to effector antigen ratio). FIG. 8C: EphA2 surface removal from L3.6pl (left) and Capan-1 (right) cells following antibody treatment. E/A ratio: EphA2 to ALCAM ratio. Data represent mean±SD (duplicate). *P<0.05, **P<0.01, and ***P<0.001.

FIG. 9 graphically illustrates the importance of the guide antigen in cell-selective cytotoxicity of exemplary antibody-drug conjugates (ADC) in accordance with some embodiments of the present disclosure. In these experiments, varying concentrations of indicated ADCs were incubated with HEK293 cells (ALCAM^{high}EphA21^{low}, where insufficient guide antigen was presence) at 37° C. for 96 hours. Cell viability was determined by Calcein-AM and normalized against a control group without ADC treatment.

FIGS. 10A-10B graphically illustrate the selection and characterization of anti-EphA2 scFvs from yeast display mutagenesis libraries. FIG. 10A: Apparent K_{D} measurement of four new EphA2 scFvs binding affinity to human recombinant EphA2 protein. In this experiment, RYRgerm is the germline version of RYR. The remaining samples were RYRgerm derivatives with high binding affinity. Apparent K_{D} values were estimated by curve-fitting of normalized MFI values. FIG. 10B: Apparent K_{D} measurement of four new EphA2 scFvs binding affinity to mouse recombinant EphA2-Fc fusion protein. Apparent K_{D} values were estimated by curve-fitting of normalized MFI values.

FIG. 11 summarizes the results of experiments performed in human prostate cancer cell lines DU145 to assess the affinities of recombinant IgG1s between the original RYR and the newly improved RYR-binding scFv RYRgerm_102919_15 described in FIGS. 10A-10B.

FIG. 12 summarizes the results of experiments performed to assess the affinities of the IgG1s described in FIGS. 10A-10B on recombinant human EphA2.

### DETAILED DESCRIPTION OF THE DISCLOSURE

The present disclosure relates generally to the fields of cell biology and immunology. More particularly, provided herein are compositions and methods for modulating internalization properties of cell-surface molecules, e.g., converting a non-internalizing cell surface antigen into an internalizing one, and vice versa. For example, in some embodiments of the disclosure, the conversion is achieved through a guide/effector system where when a set of conditions is met, the internalization property of the guide antigen is imparted onto the effector antigen. In some embodiments of the disclosure, provided are engineered antibodies each containing an antigen binding moiety specific for a cell type-selective antigen (guide-antigen) and another antigen binding moiety specific for an effector antigen, wherein the internalization property of the engineered antibody or functional fragment thereof is determined by a relative surface density ratio of the guide antigen to the effector antigen. Also provided are recombinant cells, recombinant nucleic acids encoding such engineered antibodies, as well as pharmaceutical compositions containing same. The disclosure also provides methods useful for modulating cellular internalization in a cell or a subject, as well as methods for modulating cell-type selective signaling in a subject, and/or for the treatment of health disorders and diseases, such as, for example, diseases associated with cancer, including solid tumor and hematologic malignancy.

Substantial efforts have been made to exploit antibodies to carry highly toxic payloads to infected or cancerous cells to bring and release drugs inside of cells thus acting like prodrugs. The “antibody-drug conjugate” or “ADC” approach is one such example. In this case, cell-type selective intracellular payload delivery is desired for antibody-based targeted therapy development. However, tumor-specific internalizing antigens are rare to find, and even rarer for those that are expressed at uniformly high levels. Compositions and methods disclosed herein address at least two unmet needs: (1) in targeted therapy where intracellular payload delivery is required, many tumor antigens are highly expressed but poorly internalizing. By converting them into internalizing antigens, new targeted therapies can be developed. (2) In some cases, receptor internalization is also used to shut down signaling pathways, resulting in desensitization. By converting an internalizing receptor into a non-internalizing receptor, signaling pathway can be persistently activated.

As described in greater detail below, an exemplary bispecific antibody has been constructed with a rapidly internalizing antibody binding to a tumor-associated antigen EphA2 and a non-internalizing antibody binding to a highly expressed tumor-associated antigen ALCAM. It has been observed that the overall internalization property of the bispecific is profoundly impacted by the relative surface expression level (antigen density ratio) of EphA2 vs. ALCAM. When the EphA2 to ALCAM ratio is greater than a threshold value (e.g., about 1:5), the amount of the bispecific taken into the tumor cell exceeds what is achieved by either the monoclonal internalizing antibody or a mixture of the two antibodies, showing a bispecific-dependent amplification effect where a small amount of the internalizing antigen EphA2 induces internalization of a larger amount of non-internalizing antigen ALCAM. When the ratio is below the threshold, EphA2 can be rendered non-internalizing by the presence of excess ALCAM on the same cell surface. In some illustrative experiments described below, a bispecific antibody-drug conjugate (ADC) has been constructed based on the above bispecific design, and found that the bispecific ADC is more potent than monospecific ADCs in tumor cell killing both in vitro and in vivo. Thus, the internalizing property of a cell surface antigen can be manipulated in either direction by a neighboring antigen, and this phenomenon can be exploited for therapeutic targeting.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”. In this disclosure, the use of “or” means “and/or” unless stated otherwise. Furthermore, use of the term “including” as well as other forms, such as “include”, “includes,” and “included,” is not limiting.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The term “engineered” or “recombinant” when used with reference to a cell, a nucleic acid, a protein, or a vector, indicates that the cell, nucleic acid, protein or vector has been altered through human intervention such as, for example, has been modified by or is the result of laboratory methods. Thus, for example, recombinant or engineered proteins and nucleic acids include proteins and nucleic acids produced by laboratory methods. Recombinant or engineered proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; and addition, deletion, and/or substitution of one or more of nucleic acids in the nucleic acid sequence. As such, an engineered antibody refers to a recombinant polypeptide that comprises at least an antibody fragment comprising an antigen-binding site derived from the variable domain of the heavy chain (VL) and/or light chain (VH) of an antibody and may optionally comprise the entire or part of the variable and/or constant domains of an antibody from any of the Ig classes (for example IgA, IgD, IgE, IgG, IgM and IgY). The term “engineered” when used in reference to a cell is not intended to include naturally-occurring cells but encompass cells that have been modified to include or express a polypeptide or nucleic acid that would not be present in the cell if it was not engineered.

As used herein, the term “functional fragment thereof” refers to a molecule having qualitative biological activity in common with the wild-type molecule from which the fragment or variant was derived. For example, a functional fragment of an antibody is one which retains essentially the same ability to bind to the same epitope as the antibody from which the functional fragment was derived. For example an antibody capable of binding to an epitope of a cell surface antigen may be truncated at the N-terminus and/or C-terminus, and the retention of its epitope binding activity assessed using assays known to those of skill in the art, including the exemplary assays provided herein.

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous. In addition, in the context of a polypeptide, “operably linked” refers to a physical linkage (e.g., directly or indirectly linked) between amino acid sequences (e.g., different fragments, regions, portions, or domains) to provide for a desired activity of the polypeptide. In the present disclosure, various segments, regions, or domains of the engineered antibodies of the disclosure may be operably linked to retain proper folding, processing, targeting, expression, binding, and other functional properties of the engineered antibodies in the cell. Unless stated otherwise, various regions, domains, fragments, and portions of the engineered antibodies of the disclosure are operably linked to each other. Operably linked r regions, domains, fragments, and portions of the engineered antibodies of the disclosure may be contiguous or non-contiguous (e.g., linked to one another through a linker).

The terms “percent identity”, in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity typically exists over a region that is at least about 20 amino acids or nucleotides in length, or over a region that is 10-100 amino acids or nucleotides in length, or over the entire length of a given sequence.

If necessary, sequence identity can be calculated using published techniques and widely available computer programs, such as the GCS program package (Devereux et al, Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, and unless otherwise specified, a “therapeutically effective amount” of a therapeutic agent is an amount sufficient to provide a therapeutic benefit in the treatment or management of a disease, e.g., the cancer, or to delay or minimize one or more symptoms associated with the disease. A therapeutically effective amount of a compound means an amount of therapeutic agent, alone or in combination with other therapeutic agents, which provides a therapeutic benefit in the treatment or management of the disease. The term “therapeutically effective amount” can encompass an amount that improves overall therapy of the disease, reduces or avoids symptoms or causes of the disease, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, sheep, dogs, cows, chickens, amphibians, reptiles, etc.

The term “vector” is used herein to refer to a nucleic acid molecule or sequence capable of transferring or transporting another nucleic acid molecule. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector. In some embodiments, a vector is used as a gene delivery vehicle to transfer a gene into a cell.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof Δny listed range can be recognized as sufficiently describing and enabling the same range being broken down into at least equal halves, thirds, quarters, fifths, tenths, etc. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is understood that aspects and embodiments of the disclosure described herein include “comprising,” “consisting,” and “consisting essentially of” aspects and embodiments.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Cellular Internalization for Targeted Therapy

The high specificity of monoclonal antibody is often exploited for targeted therapy development. Ideally, a potent cytotoxic attached to a cell-type selective antibody can route the cytotoxic to the target cell and preferentially accumulate in the target tissue. Antibody-drug conjugate (ADC) is a class of targeted therapeutic that has shown effectiveness in the clinic. Internalizing antibody is often desired to achieve efficient intracellular payload delivery and tumor killing, although the requirement is not absolute for certain drugs such as MMAE that can diffuse through cell membrane to cause a bystander effect.

Although conceptually straightforward, target selection in ADC is hindered by the fact that it is rare to find the so-called tumor specific antigens, and even rarer to find those that bear features desired for therapeutic targeting, e.g., uniformly expressed at high levels by cancer cells, and efficiently internalizing. Several approaches have been developed to improve antibody internalization and ADC efficacy. For example, through the biparatopic design and the consequent crosslinking effect, HER2 antigen has been targeted for improved ADC internalization. In another example, a bispecific composed of a moderately internalizing antibody arm (anti-HER2) and an internalization-inducing antibody arm (anti-CD63, anti-PRLR, or anti-APLP2) was constructed and used to improve ADC uptake. However, despite those efforts, the bispecific ADC only showed limited improvement over the parental monospecific anti-HER2 ADC, suggesting that key parameters regarding this design remain to be delineated.

One important question is if the internalization propensity of a given cell surface antigen can be impacted by its neighboring surface antigens, and if so what are the parameters that govern the conversion of a non-internalizing antigen to an internalizing antigen, and vice versa. Guide-effector bispecific systems to achieve cell-type specific signaling modulation have been reported previously. Key to these designs is the guide-to-effector ratio and the threshold of guide antigen expression. It is hypothesized that internalization can be manipulated by a guide-effector-based bispecific antibody approach. As described below, an exemplary bispecific antibody has been developed, which targeted EphA2, a rapidly internalizing antigen, and ALCAM, a non- or slowly internalizing antigen. It was found that this bispecific antibody becomes internalized when the ratio of EphA2 to ALCAM is greater than ˜1:5. It has been further showed that the bispecific effect is different from that of a simple mixture of the two monoclonal antibodies: the number of bispecific molecules delivered into the tumor cell is greater than that of the antibody mix. Therefore, the guide-effector design can cause an amplification effect, starting with a small number of seed internalizing antigen and propagating the internalizing effect cross the more abundant non-internalizing antigen. Remarkably, when the ratio of EphA2 to ALCAM is below the threshold (1:5), the internalizing EphA2 can be rendered non- or slowly internalizing by ALCAM, demonstrating that the conversion is reciprocal depending on the ratio of the guide to effector antigen. Thus when targeted by a bispecific antibody, internalization of a cell-surface antigen can be readily manipulated by its neighboring antigen, resulting in either amplified intracellular uptake or greatly retarded internalization depending on the relative abundance of the two antigens, providing an opportunity for therapeutic exploitation of cell membrane dynamics induced by the guide/effector-based bispecific disclosed herein.

As illustrated in the Examples below, a guide-effector bispecific antibody design developed previously has been adopted for cell-type selective signaling modulation to achieve cell-type selective modulation of internalization. In particular, when the guide to effector ratio crosses over a threshold (e.g., 1:5 in the EphA2/ALCAM example), a non-internalizing antigen (ALCAM) can be rendered internalizing by the bispecific antibody. When the guide to effector ratio falls below the threshold, an internalizing antigen (EphA2) can be rendered non- or slowly internalizing by the bispecific antibody. Thus in the context of bispecific targeting, the internalization behavior of a cell surface antigen is significantly impacted by its neighboring antigens, and can be readily manipulated in either direction through bispecific-based targeting of appropriately selected guide/effector pairs.

The present disclosure has relevance to therapeutic development. In the direction of converting a non-internalizing into an internalizing antigen, the present disclosure has direct relevance to ADC development. ADC is a class of anti-cancer agents that utilize the specificity of the antibody to deliver cytotoxic drug to tumor cells. Although appealing in concept, clinical development of this class of anti-cancer agents has encountered various challenges. So far, there are only 4 ADCs that are approved by the FDA for clinical use. Early problems such as drug and linker stability have been addressed but other issues remain. Although very potent drugs such as DNA chelators have been used for ADC generation, those drugs cause accumulative toxicity, restricting the therapeutic window. Microtubule inhibitors such as auristatin derivatives are less potent compared to DNA chelators and their toxicity is not accumulative except for peripheral nerve damage. Because of the reduced potency of auristatin and the limited amount of drug delivered into tumor cell, the therapeutic window remains narrow. Increasing DAR can result in more drug molecules delivered to a tumor cell in vitro, but in vivo ADCs with high DARs are rapidly cleared from the circulation, thus reducing efficacy and increasing toxicity. Site-specific conjugation achieves near uniform DAR (n=2), improves pharmacokinetic (PK) but the total number of drug molecules delivered into a tumor cell remains limited. In principle, ways to improve the therapeutic window of ADC include (1) increase cell-surface target density; and (2) improve target internalization. Both should result in a higher number of ADCs delivered intracellularly into the tumor cell. While the use of macropinocytosing antibodies for ADC construction to improve internalization was reported previously, the present disclosure provides an approach to increase target density through a guide-effector bispecific design.

As a non-limiting example, a rapidly internalizing macropinocytosing anti-EphA2 (the guide) antibody and a non/slowly internalizing anti-ALCAM (the effector) antibody are used as a model system to study the bispecific effect. It was found that when the antigen density ratio of EphA2/ALCAM is greater than a threshold (e.g., 1:5 in experimental systems described herein), a bispecific anti-ALCAMxEphA2 antibody can induce internalization of both EphA2 and ALCAM. Stated differently, the bispecific can turn a non-internalizing antigen (ALCAM) to an internalizing antigen. It was found that the bispecific ADC is more potent than either of the monospecific ADCs and furthermore the mixture of these ADCs in in vitro cytotoxicity assays, consistent with increased amount of internalized ADC delivered by the bispecific antibody. Thus, there is an amplification effect that is unique to the bispecific but not the monospecific antibodies or their mix, where a small number of internalizing antigen (the guide, EphA2), upon targeted by the bispecific antibody, can induce the internalization of a large number of non-internalizing antigen (the effector, ALCAM), resulting in greater amount of ADC and drug molecule delivered to the tumor cell compared to monoclonal ADCs and their mixture.

In addition to enhanced potency through amplified internalization, the compositions and methods disclosed herein have implication for expanding the range and type of cell-surface targets for ADC. A key challenge for current ADC is how to deliver payload specifically and in high amount to target cells. In a monoclonal antibody setting, the target antigen needs to be expressed both specifically and at a uniformly high level on the tumor surface. In practice, however, antigen with both absolute specificity and uniformly high level of expression is rare to find. As such, lineage markers, which are expressed by the tissue from which the tumor is derived, have often been used for tumor targeting. There are two limitations for those lineage markers: (1) they tend to show decreased or heterogeneous expression in late-stage cancers as they are not functionally required for tumor survival. For example, PSMA expression in late stage prostate cancer is heterogeneous and is downregulated in androgen signaling inhibitor-resistant small cell type; (2) they are often expressed in more than one normal tissue type. For example, while mesothelin is expressed by a number of tumors such as mesothelioma, ovarian cancer, and pancreatic cancer, it is also expressed by the normal mesothelium. PSMA is expressed by prostate tumor but also by a number of normal tissues. Likewise, CD19 is expressed by normal tissues other than B cells. It seems that by the monoclonal antibody approach, the target selection is rather restricted or sub-optimal. In the context of ADC, efforts have been directed to increase potency of the payload but the therapeutic window remains narrow as discussed above. An alternative approach is to identify targets that expand the difference between payloads delivered to tumor vs. normal cells. The present disclosure is particularly relevant to this approach as the guide-effector bispecific design described herein allows a large number of non-internalizing tumor-associated antigens to become internalized and thus contributing to increased intracellular delivery of ADC. The amplification effect is unique to tumor cells due to co-expression of both the guide and effector antigens.

Although the guide-effector bispecific design for cell-type selective Wnt signaling pathway modulation has been reported previously (see, e.g., Lee N K et al., Sci Rep. 2018 Jan. 15; 8(1):766.), the present disclosure expands the applicability of the bispecific approach to antigen internalization and ADC. The essence of guide-effector bispecific system disclosed herein is that the behavior of a given antigen (effector) can be shaped by the neighboring antigen (guide) when the ratio of guide to effector exceeds a threshold value. In the Wnt signaling study, when the guide/effector ratio exceeds 5-10:1, there is a thousand-fold increase in potency of the bispecific over the monoclonal antibody and the enhancement is cell-type selective. In the present disclosure, it has been showed that when the guide/effector ratio exceeds 1:5, a small number of the guide antigen (internalizing) can convert a large number of effector antigen (non-internalizing) to internalizing antigen.

Remarkably, although some experiments described below are focused on ADC and conversion of a non-internalizing antibody into an internalizing antibody, it has also been showed that the reverse is true: when the ratio of internalizing to non-internalizing antigen is below the threshold value (e.g., 1:5 in systems described herein), the internalizing antigen EphA2 is rendered slowly internalizing by the presence of the non-internalizing ALCAM. This could be useful for applications where it is desirable to have antigen remain on the cell surface to prevent degradation, and to prolong signaling functions.

There are a few recent reports of bispecific ADC with the internalizing arm binding to either a lysosomal protein or an antigen that rapidly traffics to the lysosome. In most cases, the observation is empirical and the bispecific effect is rather moderate, suggesting that key parameters affecting bispecific-induced internalization have not been fully delineated. For example, it is unclear if lysosomal antigen is required for this phenomenon. It is also unclear why the bispecific works on some cells but not the other. The present disclosure shows that a key variable in the bispecific design is the ratio of the guide to effector antigen, and there is no special feature beyond internalization that is required for the internalizing arm. The guide antigen (the internalizing arm) needs not to be a lysosomal protein to induce internalization and lysosomal trafficking. For example, in the present disclosure, micropinocytosis is exploited for selection of a macropinocytosing antibody against the cell surface antigen EphA2 as the guide to route the bispecific to the lysosomal compartment.

In summary, in the present disclosure illustrates that in the context of bispecific targeting, internalization is no longer an intrinsic property of a given antigen. Instead, antigen internalization is heavily influenced by its neighboring antigens and can be readily manipulated in either direction in a cell-type selective manner using properly selected guide/effector pairs. This bispecific-induced plasticity of cell surface dynamics can be exploited for therapeutic development.

### Compositions of the Disclosure

### Engineered Antibodies

As described in greater detail below, the present disclosure provides a new class of antibodies engineered to modulate internalization properties of cell-surface molecules, e.g., converting a non-internalizing cell surface antigen into an internalizing one, and vice versa. For example, in some embodiments of the disclosure, this conversion is achieved through a guide/effector system where when a set of conditions is met, the internalization property of the guide antigen is imparted onto the effector antigen. In some embodiments of the disclosure, an engineered antibody as disclosed herein is capable of co-engaging a cell type-selective internalizing antigen (e.g., guide antigen) and an abundantly expressed receptor (e.g., effector antigen) on a target cell.

In one aspect, some embodiments disclosed herein relate to an engineered antibody or functional fragment thereof including: a) a first antigen binding moiety capable of binding to a cell-surface guide antigen having a first rate of cellular internalization; and b) a second antigen binding moiety capable of binding to a cell-surface effector antigen having a second rate of cellular internalization, wherein the internalization property of the engineered antibody or functional fragment thereof is determined by a relative surface density ratio of the guide antigen to the effector antigen, and wherein one of the two cellular internalization rates is at least 50%, at least 70%, at least 80%, or at least 90% greater than the other rate. The term “internalization” refers to the transport of a moiety from the outside to the inside of a cell. The internalized moiety can be located in an intracellular compartment. An antigen or antibody that is “internalized” or “internalizing” refers to an antigen or antibody that is capable of being transported from the outside to the inside of a target cell.

In the engineered antibody disclosed herein, the antigen having greater cellular internalization rate is defined as a rapidly internalizing antigen, and the antigen having lower cellular internalization rate is defined as a slowly internalizing antigen. As such, in some embodiments, the internalization rate of the rapidly internalizing antigen is at least 50%, at least 70%, at least 80%, or at least 90% greater than the slowly internalizing antigen. In some particular embodiments, if >50% of surface bound antibody is internalized within 4 hours at 37° C., then the antibody is said to be rapidly internalizing. In some embodiments, if <30% of surface bound antibody is internalized after >24 hours at 37° C., then the antibody is said to be slowly internalizing. In some embodiments, if <10% of surface bound antibody is internalized after >24 hours at 37° C., then the antibody is said to be non-internalizing.

A skilled artisan will readily understand that the process of cellular internalization generally refers to the translocation of a cell-surface molecule across the plasma membrane from the cell surface to inside of the cell. Post internalization, endosomes can either traffic to lysosomes for degradation or recycle to the cell surface. The cellular internalization rate of a given cell-surface molecule provides a measurement of the kinetics of the translocation through the plasma membrane of the molecule from the surface to inside of cell. Internalization rate of antigens and antibodies can be monitored and/or measured experimentally by a number of techniques known in the art, including acid dissociation (Li N. et al., Methods Mol. Biol., 457:305-17, 2008) and toxin-killing assays (Pahara J. et al. Exp Cell Res., 316:2237-50, 2010; and Mazor et al., J Immunol. Methods, 321:41-59, 2007). Many antibody labeling technologies, dyes, and kits for antibody labeling that can be used to quantify and monitor internalization are commercially available (e.g., pHrodo iFL antibody labeling methods, reagents, and kits marketed by Thermo Fisher Scientific). For examples, the cellular internalization kinetics of the antigens and engineered antibodies of the present disclosure can be assessed and quantified by confocal microscopy or flow cytometry. For example, confocal laser scanning microscopy (CLSM) has been widely used for verify cellular internalization. Other suitable techniques, imaging flow cytometry (IFC) techniques, which provide quantitative FACS data and images of cells, can also be used to quantify cellular internalization kinetics. Additional information in this regard can be found in, for example, Ha et al., Mol Cell Proteomics, 13(12):3320-31, 2014 and Vainshtein et al., Pharm. Res. 32:286-299, 2015. In some embodiments, the cellular internalization kinetics of the engineered antibodies of the disclosure can be quantified by using the method previously described by Vainshtein et al. (Pharm Res. 2015, 32:286-299), which is herein incorporated by reference, where a confocal microscopy imaging technique is deployed to record the internalization kinetics of a fluorescence-tagged antibody in live cells and a quantitative image-analysis algorithm is used for the determination of an internalization rate constant (Kᵢₙₜ). In some embodiments, the internalization rate constant Kᵢₙₜ of an engineered antibody as disclosed herein is calculated from internalization time course by curve fitting of the data using the equation: S_{cyt}(t)=S_{0,cyt}+(1−e−^{Kint.t})·S_{max,cyt}; wherein S_{cyt}(t) is the cytoplasmic fluorescence signal at time t; S_{0,cyt} and S_{max,cyt} are initial cytoplasmic fluorescence signal and maximal signal, respectively (see, Vainshtein et al. 2015).

Designation of the antigen binding moiety capable of binding to a cell-surface guide antigen as the “first” antigen binding moiety and the antigen binding moiety capable of binding to a cell-surface effector antigen as the “second” antigen binding moiety is not intended to imply any particular structural arrangement of the “first” and “second” antigen binding moieties within the engineered antibody. By way of non-limiting example, in some embodiments of the disclosure, the engineered antibody may include an N-terminal portion including an antigen binding moiety capable of binding to a cell-surface guide antigen and a C-terminal portion including an antigen binding moiety capable of binding to a cell-surface effector antigen. In other embodiments, the engineered antibody may include an N-terminal portion including an antigen binding moiety capable of binding to a cell-surface effector antigen and a C-terminal portion including an antigen binding moiety capable of binding to a cell-surface guide antigen.

As described in greater detail below, the first and/or second antigen binding moiety is multispecific, e.g. capable of binding to more than one antigen, e.g., more than two, more than three, more than four, more than five, or more than six different antigens. For example, in some embodiments, the first antigen binding moiety can be configured to have dual specificity, i.e., is capable of binding to two guide antigens. In some embodiments, the second antigen binding moiety can be configured to have dual specificity, i.e., is capable of binding to two effector antigens. Additional information regarding this two-in-one antibody design can be found in, for example, Schaefer G. et al., Cancer Cell. 2011 Oct. 18; 20(4):472-86; and Lee C V et al., MAbs. 2014; 6(3):622-627.

In addition or alternatively, the engineered antibody may include more than one antigen binding moiety capable of binding to a cell-surface guide antigen, and/or more than one antigen binding moiety capable of binding to a cell-surface effector antigen. Accordingly, in some embodiments, the engineered antibody may include multiple antigen binding moieties each of which is capable of binding to a cell-surface guide antigen. In some embodiments, the engineered antibody may include multiple antigen binding moieties each of which is capable of binding to a cell-surface effector antigen. In some embodiments, the engineered antibody include multiple antigen binding moieties each of which is capable of binding to a cell-surface guide antigen and multiple antigen binding moieties each of which is capable of binding to a cell-surface effector antigen.

In accordance with the present disclosure, it is possible to induce rapid internalization of a slowly internalizing antigen by operably linked an antigen binding moiety specific for such a slowly internalizing antigen with another antigen binding moiety specific for a rapidly internalizing antigen. In some embodiments, it is possible to induce slow internalization of a rapidly internalizing antigen by operably linking an antigen binding moiety specific for such rapidly internalizing antigen with another antigen binding moiety specific for a slowly internalizing antigen.

In some embodiments, the internalization property of the engineered antibody as disclosed herein is converted from internalizing to non-internalizing. In some embodiments, the internalization property of the guide antigen and/or the effector antigen is converted from internalizing to non-internalizing. In some embodiments, the internalization property of an internalizing antigen (e.g., guide antigen or effector antigen) is converted from internalizing to non-internalizing by using an engineered antibody as disclosed herein which includes an antigen binding moiety specific for such an internalizing antigen operably linked with another antigen binding moiety specific for a non-internalizing antigen. For example, in some embodiments, the internalization property of an internalizing guide antigen is converted from internalizing to non-internalizing by using an engineered antibody as disclosed herein which includes an antigen binding moiety specific for the internalizing guide antigen operably linked with another antigen binding moiety specific for a non-internalizing effector antigen. In some embodiments, the internalization property of an internalizing effector antigen is converted from internalizing to non-internalizing by using an engineered antibody as disclosed herein which includes an antigen binding moiety specific for the internalizing effector antigen operably linked with another antigen binding moiety specific for a non-internalizing guide antigen.

In some other embodiments, the internalization property of the engineered antibody disclosed herein is converted from non-internalizing to internalizing. In some embodiments, the internalization property of a non-internalizing antigen (e.g., guide antigen or effector antigen) is converted from non-internalizing to internalizing. In some other embodiments, the internalization property of a non-internalizing guide antigen is converted from non-internalizing to internalizing by using an engineered antibody as disclosed herein which includes an antigen binding moiety specific for such non-internalizing guide antigen operably linked with another antigen binding moiety specific for an internalizing effector antigen. In some other embodiments, the internalization property of a non-internalizing effector antigen is converted from non-internalizing to internalizing by using an engineered antibody as disclosed herein which includes an antigen binding moiety specific for such non-internalizing effector antigen operably linked with another antigen binding moiety specific for an internalizing guide antigen.

In some embodiments, the guide antigen has a rate of cellular internalization that is greater than the cellular internalization rate of the effector antigen, in which case the guide antigen is a rapidly internalizing antigen and the effector antigen is a slowly internalizing antigen. In some embodiments, the guide antigen has a rate of cellular internalization of at least about 50% greater than the cellular internalization rate of the effector antigen. In some embodiments, the guide antigen has a rate of cellular internalization of at least about 50%, 60%, 70%, 80%, or 90% greater than the cellular internalization rate of the effector antigen. In some embodiments, the engineered antibody of the disclosure increases the internalization rate of the slowly internalizing antigen (e.g., effector antigen) by at least 10%, 20%, 30%, 40%, 50%, 60%, 70%, 80%, 90%, 99% or 99.9% as compared to a control (e.g., a monospecific antibody comprising only the slowly internalizing antigen) by operably linked an antigen binding moiety specific for the slowly internalizing antigen with another antigen binding moiety specific for a rapidly internalizing antigen (e.g. guide antigen). In some embodiments, the engineered antibody of the disclosure reduces the internalization rate of the rapidly internalizing antigen (e.g., guide antigen) by at least 10%, 20%, 30%, 40%, 50%, 60%, 70%, 80%, 90%, 99% or 99.9% as compared to a control (e.g., a monospecific antibody comprising only the rapidly internalizing antigen) by operably linking an antigen binding moiety specific for the rapidly internalizing antigen with another antigen binding moiety specific for a slowly internalizing antigen (e.g., effector antigen).

In some embodiments, the effector antigen has a rate of cellular internalization that is greater than the cellular internalization rate of the guide antigen, in which case the effector antigen is a rapidly internalizing antigen and the guide antigen is a slowly internalizing antigen. In some embodiments, the effector antigen has a rate of cellular internalization of at least about 50% greater than the cellular internalization rate of the guide antigen. In some embodiments, the effector antigen has a rate of cellular internalization of at least about 50%, 60%, 70%, 80%, or 90% greater than the cellular internalization rate of the guide antigen. In some embodiments, the engineered antibody of the disclosure increases the internalization rate of the slowly internalizing antigen (e.g., guide antigen) by at least 10%, 20%, 30%, 40%, 50%, 60%, 70%, 80%, 90%, 99% or 99.9% as compared to a control (e.g., a monospecific antibody comprising only the slowly internalizing antigen) by operably linked an antigen binding moiety specific for the slowly internalizing antigen with another antigen binding moiety specific for a rapidly internalizing antigen (e.g. effector antigen). In some embodiments, the engineered antibody of the disclosure reduces the internalization rate of the rapidly internalizing antigen (e.g., effector antigen) by at least 10%, 20%, 30%, 40%, 50%, 60%, 70%, 80%, 90%, 99% or 99.9% as compared to a control (e.g., a monospecific antibody comprising only the rapidly internalizing antigen) by operably linking an antigen binding moiety specific for the rapidly internalizing antigen with another antigen binding moiety specific for a slowly internalizing antigen (e.g., guide antigen). In some embodiments, the cell-surface guide antigen is an internalizing cell surface antigen. In some embodiments, the cell-surface effector antigen is a non-internalizing cell surface antigen.

The internalization property of the engineered antibody as disclosed herein or functional fragment thereof is determined by a relative surface density ratio of the guide antigen to the effector antigen. One skilled in the art will readily appreciate that the surface density a given molecule such as an antigen or a polypeptide refers to a number of the antigen or polypeptide that is measured and/or estimated on a given surface area. For example, a density of antigen present on a cell surface can be presented as about 10,000 copies per cell, meaning the measured and/or estimated number of the antigen molecules present on the cell surface is about 10,000. Many techniques, systems, assays, and procedures for determining and/or measuring the density of molecules present on the cell surface are known in the art. Additional information in this regard can be found at Example 12 below and in, e.g., Lee N K et al., Sci. Rep. January 15; 8(1):766, 2018, and Sherbenou, D W et al., J. Clin. Invest. 2016 Nov. 14. In some embodiments of the present disclosure, the surface density of the guide antigen and the effector antigen are measured. The results are then compiled and interpreted as a single ratio between the surface density of the guide antigen and the surface density of the effector antigen. A decision rule may state that any score above a given threshold indicates internalization of the engineered antibody, while a score below the threshold indicates the lack of internalization, e.g., non-internalization.

In some embodiments, these scores may be compared to threshold values, such that scores above a threshold value are indicative of an increased or decreased internalization as indicated by an engineered antibody. The surface densities, ratios, and appropriate threshold for each guide/effector pair may be determined by collecting data on a small set of samples from both internalizing and non-internalizing antigens, then using a linear model to separate them. The linear model may be generated via a statistical method such as logistic regression or support vector machines with a linear kernel function, or the linear model may be generated by inspection.

In some embodiments, the relative surface density ratio of the guide antigen to the effector antigen is greater than a threshold value. In some embodiments, the threshold value is about 1:1, about 1:2, about 1:3, about 1:4, about 1:5, about 1:10, about 1:20, or about 1:30. Accordingly, in some embodiments, the relative surface density ratio of the guide antigen to the effector antigen is greater than about 1:1, about 1:2, about 1:3, about 1:4, about 1:5, about 1:10, about 1:20, or about 1:30. In some embodiments, the threshold value is about 1:5. In some embodiments, the relative surface density ratio of the guide antigen to the effector antigen is lower than a threshold value. In some embodiments, the threshold value is about 1:1, about 1:2, about 1:3, about 1:4, about 1:5, about 1:10, about 1:20, or about 1:30. Accordingly, in some embodiments, the relative surface density ratio of the guide antigen to the effector antigen is lower than about 1:1, about 1:2, about 1:3, about 1:4, about 1:5, about 1:10, about 1:20, or about 1:30. In some embodiments, the threshold value is about 1:5.

In some other embodiments, the relative surface density ratio of the effector antigen to the guide antigen is greater than a threshold value. In some embodiments, the threshold value is about 1:1, about 1:2, about 1:3, about 1:4, about 1:5, about 1:10, about 1:20, or about 1:30. Accordingly, in some embodiments, the relative surface density ratio of the effector antigen to the guide antigen is greater than about 1:1, about 1:2, about 1:3, about 1:4, about 1:5, about 1:10, about 1:20, or about 1:30. In some embodiments, the threshold value is about 1:5. In some other embodiments, the relative surface density ratio of the effector antigen to the guide antigen is lower than a threshold value. In some embodiments, the threshold value is about 1:1, about 1:2, about 1:3, about 1:4, about 1:5, about 1:10, about 1:20, or about 1:30. Accordingly, in some embodiments, the relative surface density ratio of the effector antigen to the guide antigen is lower than about 1:1, about 1:2, about 1:3, about 1:4, about 1:5, about 1:10, about 1:20, or about 1:30. In some embodiments, the threshold value is about 1:5.

As used herein, the term “antigen binding moiety” refers to a polypeptide that specifically binds to an antigenic determinant, e.g., an antigen. In some embodiments, an antigen binding moiety is able to direct the entity to which it is attached (e.g. an engineered antibody comprising a second antigen binding moiety) to a target site, for example to a specific cell type, such as a type of tumor cell or tumor stroma bearing the antigenic determinant. For example, antibodies, antibody fragments, antibody derivatives, antibody-like scaffolds and alternative scaffolds comprise at least one antigen binding moiety. Antigen-binding moieties can also be incorporated into single domain antibodies, maxibodies, minibodies, intrabodies, diabodies, triabodies, tetrabodies, v-NAR and scFv. Accordingly, in some embodiments, the first antigen binding moiety and the second antigen binding moiety are independently selected from the group consisting of an antigen-binding fragment (Fab), a single-chain variable fragment (scFv), a full-length immunoglobulin, a nanobody, a single domain antibody (sdAb), a VNAR domain, and a VHH domain, a diabody, or a functional fragment thereof. In some embodiments, the first antigen binding moiety and/or the second antigen binding moiety are monovalent. In some embodiments, the first antigen-binding moiety and/or the second antigen binding moiety are multivalent, e.g., including more than one antigen-binding site. In some embodiments, the first antigen-binding moiety and/or the second antigen binding moiety are monospecific. In some embodiments, the first antigen binding moiety and/or the second antigen-binding moiety are multispecific, e.g., including antigen-binding sites with specific binding activity for at least two different target antigens, e.g., at least two, at least three, at least four, at least five target antigens.

The term “antigen-binding site” as used herein refers to a portion of an antigen binding moiety that is responsible for the specific binding between the antigen binding moiety and an antigen determinant. An antigen-binding site may be a single domain, for example an epitope-binding domain, or it may be a paired VH/VL domains as can be found on a standard antibody. Accordingly, in some embodiments, the antigen-binding site of an antibody or a fragment thereof as described herein is formed by amino acid residues of the N-terminal variable regions of the heavy chain (VH) and the light chain (VL). Generally, the variable regions of the VH and the VL each comprise three hypervariable regions, termed complementary determining regions (CDR). The 3 CDRs of the VH (termed HCDR1, HCDR2, and HCDR3) and the 3 CDRs of the VL (termed LCDR1, LCDR2, and LCDR3) are three-dimensionally disposed relative to each other to form an antigen binding surface. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.). While any suitable numbering system may be used to designated CDR regions, in the absence of any other indication, the sequences of the CDRs of the engineered antibodies of the disclosure, according to Kabat definition system, have been summarized in Tables 4 and 5 below.

The binding of the first and second antigen binding moieties to their respective target can be either in a competitive or non-competitive fashion with a natural ligand of the target. Accordingly, in some embodiments of the disclosure, the binding of the first and/or second antigen binding moieties to their respective target can be ligand-blocking. In some other embodiments, the binding of the first and/or second antigen binding moieties to their respective target does not block binding of the natural ligand. In some embodiments of the disclosure, the engineered antibody includes a first amino acid sequence encoding the first antigen binding moiety, which is linked to a second amino acid sequence encoding the second antigen binding moiety with which it is not naturally linked in nature. The amino acid sequences may normally exist in separate proteins that are brought together in the fusion polypeptide or they may normally exist in the same protein but are placed in a new arrangement in the fusion polypeptide. Amino acid sequences encoding the first and second antigen moieties may be created, for example, by chemical synthesis, or by creating and translating a polynucleotide in which the peptide regions are encoded in the desired relationship.

In some embodiments, the first antigen binding moiety is directly linked to the second antigen binding moiety. In some embodiments, the first antigen binding moiety is directly linked to the second antigen binding moiety via at least one covalent bond. In some embodiments, the first antigen binding moiety is directly linked to the second antigen binding moiety via at least one peptide bond. In some embodiments, the C-terminal amino acid of the first antigen binding moiety can be operably linked to the N-terminal amino acid of the second antigen binding moiety. Alternatively, the N-terminal amino acid of the first antigen binding moiety can be operably linked to the C-terminal amino acid of the second antigen binding moiety.

In some embodiments, the first antigen binding moiety is operably linked to the second antigen binding moiety via a linker. There is no particular limitation on the linkers that can be used in the engineered antibodies described herein. In some embodiments, the linker is a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

In some embodiments, the first antigen binding moiety is operably linked to the second antigen binding moiety via a linker peptide sequence. In principle, there are no particular limitations to the length and/or amino acid composition of the linker peptide sequence. In some embodiments, any arbitrary single-chain peptide comprising about one to about 100 amino acid residues (e.g., 2, 3, 4, 5, 6, 7, 8, 9, 10, 11, 12, 13, 14, 15, 16, 17, 18, 19, 20, etc. amino acid residues) can be used as a peptide linker. In some embodiments, the linker peptide sequence includes about 5 to about 50, about 10 to about 60, about 20 to about 70, about 30 to about 80, about 40 to about 90, about 50 to about 100, about 60 to about 80, about 70 to about 100, about 30 to about 60, about 20 to about 80, about 30 to about 90 amino acid residues. In some embodiments, the linker peptide sequence includes about 1 to about 10, about 5 to about 15, about 10 to about 20, about 15 to about 25, about 20 to about 40, about 30 to about 50, about 40 to about 60, about 50 to about 70 amino acid residues. In some embodiments, the linker peptide sequence includes about 40 to about 70, about 50 to about 80, about 60 to 8 about 0, about 70 to about 90, or about 80 to about 100 amino acid residues. In some embodiments, the linker peptide sequence includes about 1 to about 10, about 5 to about 15, about 10 to about 20, about 15 to about 25 amino acid residues.

In some embodiments, the length and amino acid composition of the linker peptide sequence can be optimized to vary the orientation and/or proximity of the first and second antigen binding moieties to one another to achieve a desired activity of the engineered antibody. In some embodiments, the orientation and/or proximity of the first and second antigen binding moieties to one another can be varied as a “tuning” tool to achieve a tuning effect that would enhance or reduce one or more desired activities of the engineered antibody. For examples, in some embodiments, the orientation and/or proximity of the first and second antigen binding moieties to one another can be optimized to create a competitive, partially competitive, or non-competitive versions of the engineered antibodies. In certain embodiments, the linker contains only glycine and/or serine residues (e.g., glycine-serine linker).

Antigens

In some embodiments, the engineered antibodies of the present disclosure, such as bispecific antibodies, can have binding specificity to two separate cell surface antigens, one of which has a more rapidly internalizing rate than that of the other. A bispecific antibody can include at least two components, a first component and a second component, each of which binds to their respective antigen, e.g., a first cell-type-associated antigen (guide antigen) and a second antigen associated with a target signaling pathway (effector antigen), respectively. The first component can include a first antigen binding moiety for the first antigen and the second component can include a second antigen binding moiety for the second antigen. Such bispecific antibody allows for an increased potency to inhibit the target signaling pathway compared to a non-targeted antibody (e.g., an antibody that does not have binding specificity to the effector antigen), and importantly, allows for cell-type specific inhibition.

Non limiting examples of cell surface antigens suitable for the engineered antibodies of the disclosure include activated leukocyte cell adhesion molecule (ALCAM), neural cell adhesion molecule (NCAM), calcium-activated chloride channel 2 (CaCC), carbonic anhydrase IX, carcinoembroyonic antigen (CEA), cathepsin G, CD19, CD20, CD22, CD30, CD33, CD38, CD44, CD44v6, CD46, CD52, CD71, CD73, CD272, CD276, B-cell maturation antigen (BCMA), epithelial cell adhesion molecule (EpCAM), ephrin type-A receptor 2 (EphA2), ephrin type-A receptor 3 (EphA3), ephrin type-A receptor 4 (EphA4), ephrin B2, receptor tyrosine kinase like orphan receptor 1 (ROR1), folate receptor, FLT3 (CD135), KIT (CD117), CD213A2, IL-1Ra, PRSS21, VEGFR2, CD24, PDGFR-beta, SSEA-4, epidermal growth factor receptor (EGFR), Erb-B2 receptor tyrosine kinase 2 (ErbB2), Erb-B2 receptor tyrosine kinase 3 (ErbB3), Erb-B2 receptor tyrosine kinase 4 (ErbB4), folate binding proteins (folate receptors), ganglioside, gangliosides, gp100, gpA33, immature laminin receptor, intercellular adhesion molecule 1 (ICAM-1), Lewis-Y, mesothelin, prostate stem cell antigen (PSCA), mucin 16 (MUC16 or CA-125), mucin 1 cell-surface associated (MUC1), mucin 2 oligomeric mucus gel-forming (MUC2), mucins, prostate membrance specific antigen (PSMA), TEM1/CD248, TEM7R, CLDN6, thyroid stimulating hormone receptor (TSHR), GPRC5D, CD97, CD179a, anaplastic lymphoma kinase (ALK or CD246), immunoglobulin lambda like polypeptide 1 (IGLL1), P-selectin, c-Met, fibroblast growth factor receptors (FGFRs), insulin-like growth factor 1 receptor (IGF-1R), tumor-associated calcium signal transducer 2 (Trop-2), and tumor associated glycoprotein 72 (TAG-72). In some embodiments, the cell surface antigens include ICAM-1, EphA2, and ALCAM.

Guide Antigens

In some embodiments, guide antigen that is recognized by an engineered antibody of the disclosure is a molecule serving as a cell-type associated antigen. The cell-type associated antigen generally refers to a molecule in which the expression level is substantially higher in a certain type of cell(s) of interest (“target cell(s)”) as compared to a non-target cell(s). In some embodiments, the guide antigen can be any cell surface antigen that is overexpressed on the target cell. For example, there are molecules that are overexpressed in cancer cells such as intercellular adhesion molecule 1 (ICAM-1), EphA2, and activated leukocyte. In some embodiments, the guide antigen is a cell adhesion molecule (ALCAM) and these molecules may be considered as cancer-associated antigen or tumor-associated antigens.

In some embodiments, the guide antigen is a cancer-associated antigen. Non-limiting examples of cancer-associated antigens suitable for the compositions and methods of the disclosure include CD19, CD22, HER2 (ErbB2/neu), mesothelin, PSCA, CD123, CD30, CD71, CD171, CS-1, CLECL1, CD33, EGFRvIII, GD2, GD3, BCMA, PSMA, receptor tyrosine kinase like orphan receptor 1 (ROR1), folate receptor, FLT3 (CD135), TAG72, CD38, CD44v6, CD46, CEA, EpCAM, CD272, B7H3 (CD276), KIT (CD117), CD213A2, IL-1Ra, PRSS21, VEGFR2, CD24, PDGFR-beta, SSEA-4, CD20, MUC1, MUC16, EGFR, ErbB2, ErbB3, ErbB4, NCAM, prostatic acid phosphatase (PAP), ephrin B2, fibroblast activation protein (FAP), EphA2, c-Met, fibroblast growth factor receptors (FGFRs), insulin-like growth factor 1 receptor (IGF-1R), GM3, TEM1/CD248, TEM7R, CLDN6, thyroid stimulating hormone receptor (TSHR), GPRC5D, CD97, CD179a, anaplastic lymphoma kinase (ALK or CD246), and immunoglobulin lambda like polypeptide 1 (IGLL1). In some embodiments, the engineered antibody or functional fragment disclosed herein includes an antigen binding moiety capable of binding to an EphA2 expressed on the surface of a cell.

In some embodiments, by specifically recognizing and binding to the cell-type associated antigen (e.g., guide antigen), an engineered antibody of the disclosure can be recruited to a target cell that is associated with the guide antigen, resulting in, e.g., a modulation of a signaling pathway in the target cell. In some embodiments, the guide antigen of an engineered antibody of the present disclosure not only serves as a cell-type selector but also as a potency enhancer, resulting in, e.g., a potent and selective inhibition of a target signaling pathway. In some embodiments, there is a threshold value of the expression for the guide antigen on the surface of a target cell, which results in enhancement in (1) the binding affinity of the engineered antibody to the target cell and (2) the occupancy of the effector antigen by the engineered antibody.

Effector Antigens

In some embodiments, an effector antigen that is recognized by an engineered antibody described herein is a molecule associated with a cellular activity or function such as, a signaling pathway. In some embodiments, an effector antigen is expressed on the surface of a cell of interest. In some embodiments, an effector antigen that is recognized by an engineered antibody described herein is a molecule associated with a signaling pathway of interest (e.g., a target signaling pathway). In some cases, the effector antigen includes a tumor antigen (e.g., a tumor-associated antigen or a tumor-specific antigen). Non-limiting examples of effector antigens suitable for the engineered antibodies of the disclosure include ALCAM, EpCAM, Folate binding proteins, PSMA, PSCA, Mesothelin, CD19, CD20, CD22, CD30, CD33, CD38, CD44, CD46, ICAM-1, CD55, CD59, CD70, CD71, CD73, CD97, BCMA, CD272, CD276, MUC1, MUC16, NCAM, CD24, EphA2, EphA3, EphA4, Ephrin B2, CEA, c-Met, FGFRs, IGF-1R, VEGFRs, PDGFRs, Trop-2, TAG-72, P-selectin. Further examples of suitable effector antigens are further described below and include EGFR, ErbB2, ErbB3, and ErbB4. In some embodiments, the engineered antibody or functional fragment disclosed herein includes an antigen binding moiety capable of binding to an ALCAM expressed on the surface of a cell.

In some particular embodiments, the engineered antibody or functional fragment disclosed herein includes a first antigen binding moiety capable of binding to an EphA2 expressed on the surface of a cell; and a second antigen binding moiety capable of binding to an ALCAM expressed on the surface of the same cell. In some embodiments, the surface density ratio of EphA2 to ALCAM is greater than a threshold value. In some embodiments, the surface density ratio of EphA2 to ALCAM is greater than a threshold value of about 1:1, about 1:2, about 1:3, about 1:4, about 1:5, about 1:10, about 1:20, or about 1:30. In some embodiments, the surface density ratio of EphA2 to ALCAM is greater than a threshold value of about 1:5.

In some embodiments, the engineered antibody or functional fragment thereof as described herein includes an amino acid sequence having at least 80% sequence identity to any one of the amino acid sequences disclosed herein. In some embodiments, the engineered antibody or functional fragment thereof as described herein includes an amino acid sequence having at least 80%, at least 90%, at least 95%, at least 96%, at least 97, at least 98%, or at least 99% sequence identity to any one of the amino acid sequences disclosed herein. In some embodiments, the engineered antibody or functional fragment thereof as described herein includes an amino acid sequence having at least 80%, at least 90%, at least 95%, at least 96%, at least 97, at least 98%, or at least 99% sequence identity to any one of the amino acid sequences identified in Table 4. In some embodiments, the engineered antibody or functional fragment thereof as described herein includes an amino acid sequence having 100% sequence identity to any one of the amino acid sequences identified in Table 4. In some embodiments, the engineered antibody or functional fragment thereof as described herein includes an amino acid sequence corresponding to any one of the amino acid sequences identified in Table 4, wherein one, two, three, four, or five of the amino acid residues in the amino acid sequence is substituted by a different amino acid residue.

First Antigen Binding Moieties

As outlined above, various embodiments and aspects of the present disclosure include an engineered antibody including a first antigen binding moiety capable of binding to a cell-surface guide antigen. In some embodiments, the first antigen binding moiety includes a heavy chain variable (VH) region having at least 80%, at least 90%, at least 95%, at least 96%, at least 97, at least 98%, or at least 99% sequence identity to a VH sequence identified in Table 4. In some embodiments, the first antigen binding moiety includes a VH region having at least 80%, at least 90%, at least 95%, at least 96%, at least 97, at least 98%, or at least 99% sequence identity to SEQ ID NO: 81. In some embodiments, the first antigen binding moiety includes a VH region having at least 80%, at least 90%, at least 95%, at least 96%, at least 97, at least 98%, or at least 99% sequence identity to SEQ ID NO: 96. In some embodiments, the first antigen binding moiety includes a VH region with an amino acid sequence having 100% sequence identity to a VH sequence identified in Table 4. In some embodiments, the first antigen binding moiety includes a VH region with an amino acid sequence having 100% sequence identity to SEQ ID NO: 81. In some embodiments, the first antigen binding moiety includes a VH region with an amino acid sequence having 100% sequence identity to SEQ ID NO: 96. In some embodiments, the first antigen binding moiety includes a VH region having an amino acid sequence corresponding to any one of the VH sequences identified in Table 4, wherein one, two, three, four, or five of the amino acid residues in the amino acid sequence is substituted by a different amino acid residue. In some embodiments, the first antigen binding moiety includes a VH region having an amino acid sequence corresponding to SEQ ID NO: 81, wherein one, two, three, four, or five of the amino acid residues in the amino acid sequence of SEQ ID NO: 81 is substituted by a different amino acid residue. In some embodiments, the first antigen binding moiety includes a VH region having an amino acid sequence corresponding to SEQ ID NO: 96, wherein one, two, three, four, or five of the amino acid residues in the amino acid sequence of SEQ ID NO: 96 is substituted by a different amino acid residue.

In some embodiments, the VH region of the first antigen binding moiety includes three CDRs (e.g., HCDR1, HCDR2, and HCDR3) as identified in each of the VH sequences disclosed in the Sequence Listing. In some embodiments, the HCDR1 of the first antigen binding moiety includes the sequence of SEQ ID NO: 104. In some embodiments, the HCDR2 of the first antigen binding moiety includes the sequence of SEQ ID NO: 105. In some embodiments, the HCDR3 of the first antigen binding moiety includes the sequence of SEQ ID NO: 106 or SEQ ID NO: 110. In some embodiments, the HCDR1, HCDR2, and HCDR3 of the VH region of the first antigen binding moiety includes the sequences of SEQ ID NO: 104, SEQ ID NO: 105, and SEQ ID NO: 106, respectively. In some embodiments, the HCDR1, HCDR2, and HCDR3 of the VH region of the first antigen binding moiety includes the sequences of SEQ ID NO: 104, SEQ ID NO: 105, and SEQ ID NO: 110, respectively. In some embodiments, the VH region of the first antigen binding moiety includes three HCDRs as identified in each of the VH sequences disclosed in the Sequence Listing, wherein one, two, three, four, or five of the amino acid residues in at least one of the HCDRs is substituted by a different amino acid residue. In some embodiments, the HCDR1 of the first antigen binding moiety includes the sequence of SEQ ID NO: 104, wherein one, two, three, four, or five of the amino acid residues in SEQ ID NO: 104 is substituted by a different amino acid residue. In some embodiments, the HCDR2 of the first antigen binding moiety includes the sequence of SEQ ID NO: 105, wherein one, two, three, four, or five of the amino acid residues in SEQ ID NO: 105 is substituted by a different amino acid residue. In some embodiments, the HCDR3 of the first antigen binding moiety includes the sequence of SEQ ID NO: 106, wherein one, two, three, four, or five of the amino acid residues in SEQ ID NO: 106 is substituted by a different amino acid residue. In some embodiments, the HCDR3 of the first antigen binding moiety includes the sequence of SEQ ID NO: 110, wherein one, two, three, four, or five of the amino acid residues in SEQ ID NO: 110 is substituted by a different amino acid residue.

In some embodiments, the first antigen binding moiety includes a light chain variable (VL) region having at least 80%, at least 90%, at least 95%, at least 96%, at least 97, at least 98%, or at least 99% sequence identity to a VL sequence identified in Table 4. In some embodiments, the first antigen binding moiety includes a VL region having at least 80%, at least 90%, at least 95%, at least 96%, at least 97, at least 98%, or at least 99% sequence identity to SEQ ID NO: 82. In some embodiments, the first antigen binding moiety includes a VL region having at least 80%, at least 90%, at least 95%, at least 96%, at least 97, at least 98%, or at least 99% sequence identity to SEQ ID NO: 97. In some embodiments, the first antigen binding moiety includes a VL region with an amino acid sequence having 100% sequence identity to a VL sequence identified in Table 4. In some embodiments, the first antigen binding moiety includes a VL region with an amino acid sequence having 100% sequence identity to SEQ ID NO: 82. In some embodiments, the first antigen binding moiety includes a VL region with an amino acid sequence having 100% sequence identity to SEQ ID NO: 97. In some embodiments, the first antigen binding moiety includes a VL region having an amino acid sequence corresponding to any one of the VL sequences identified in Table 4, wherein one, two, three, four, or five of the amino acid residues in the amino acid sequence is substituted by a different amino acid residue. In some embodiments, the first antigen binding moiety includes a VL region having an amino acid sequence corresponding to SEQ ID NO: 82, wherein one, two, three, four, or five of the amino acid residues in the amino acid sequence of SEQ ID NO: 82 is substituted by a different amino acid residue. In some embodiments, the first antigen binding moiety includes a VL region having an amino acid sequence corresponding to SEQ ID NO: 97, wherein one, two, three, four, or five of the amino acid residues in the amino acid sequence of SEQ ID NO: 97 is substituted by a different amino acid residue.

In some embodiments, the VL region of the first antigen binding moiety includes three CDRs (e.g., LCDR1, LCDR2, and LCDR3) as identified in each of the VL sequences disclosed in the Sequence Listing. In some embodiments, the LCDR1 of the first antigen binding moiety includes the sequence of SEQ ID NO: 107. In some embodiments, the LCDR2 of the first antigen binding moiety includes the sequence of SEQ ID NO: 108. In some embodiments, the LCDR3 of the first antigen binding moiety includes the sequence of SEQ ID NO: 109. In some embodiments, the LCDR1, LCDR2, and LCDR3 of the VL region of the first antigen binding moiety includes the sequences of SEQ ID NO: 107, SEQ ID NO: 108, and SEQ ID NO: 109, respectively. In some embodiments, the first antigen binding moiety includes a VL region having an amino acid sequence corresponding to any one of the VL sequences identified in Table 4, wherein one, two, three, four, or five of the amino acid residues in the amino acid sequence is substituted by a different amino acid residue. In some embodiments, the first antigen binding moiety includes a VL region having an amino acid sequence corresponding to SEQ ID NO: 82, wherein one, two, three, four, or five of the amino acid residues in the amino acid sequence of SEQ ID NO: 82 is substituted by a different amino acid residue. In some embodiments, the first antigen binding moiety includes a VL region having an amino acid sequence corresponding to SEQ ID NO: 97, wherein one, two, three, four, or five of the amino acid residues in the amino acid sequence of SEQ ID NO: 97 is substituted by a different amino acid residue.

In some embodiments, the VL region of the first antigen binding moiety includes three CDRs (e.g., LCDR1, LCDR2, and LCDR3) as identified in each of the VL sequences disclosed in the Sequence Listing, wherein one, two, three, four, or five of the amino acid residues in at least one of the LCDRs is substituted by a different amino acid residue. In some embodiments, the LCDR1 of the first antigen binding moiety includes the sequence of SEQ ID NO: 107, wherein one, two, three, four, or five of the amino acid residues in SEQ ID NO: 107 is substituted by a different amino acid residue. In some embodiments, the LCDR2 of the first antigen binding moiety includes the sequence of SEQ ID NO: 108, wherein one, two, three, four, or five of the amino acid residues in SEQ ID NO: 108 is substituted by a different amino acid residue. In some embodiments, the LCDR3 of the first antigen binding moiety includes the sequence of SEQ ID NO: 109, wherein one, two, three, four, or five of the amino acid residues in SEQ ID NO: 109 is substituted by a different amino acid residue.

Second Antigen Binding Moieties

As outlined above, various embodiments and aspects of the present disclosure include an engineered antibody including a second antigen binding moiety capable of binding to a cell-surface effector antigen. In some embodiments, the second antigen binding moiety includes a VH region having at least 80%, at least 90%, at least 95%, at least 96%, at least 97, at least 98%, or at least 99% sequence identity to a VH sequence identified in Table 4. In some embodiments, the second antigen binding moiety includes a VH region having at least 80%, at least 90%, at least 95%, at least 96%, at least 97, at least 98%, or at least 99% sequence identity to SEQ ID NO: 73. In some embodiments, the second antigen binding moiety includes a VH region having at least 80%, at least 90%, at least 95%, at least 96%, at least 97, at least 98%, or at least 99% sequence identity to SEQ ID NO: 75. In some embodiments, the second antigen binding moiety includes a VH region with an amino acid sequence having 100% sequence identity to a VH sequence identified in Table 4. In some embodiments, the second antigen binding moiety includes a VH region with an amino acid sequence having 100% sequence identity to SEQ ID NO: 73. In some embodiments, the second antigen binding moiety includes a VH region with an amino acid sequence having 100% sequence identity to SEQ ID NO: 75. In some embodiments, the second antigen binding moiety includes a VH region having an amino acid sequence corresponding to any one of the VH sequences identified in Table 4, wherein one, two, three, four, or five of the amino acid residues in the amino acid sequence is substituted by a different amino acid residue. In some embodiments, the second antigen binding moiety includes a VH region having an amino acid sequence corresponding to SEQ ID NO: 73, wherein one, two, three, four, or five of the amino acid residues in the amino acid sequence of SEQ ID NO: 73 is substituted by a different amino acid residue. In some embodiments, the second antigen binding moiety includes a VH region having an amino acid sequence corresponding to SEQ ID NO: 75, wherein one, two, three, four, or five of the amino acid residues in the amino acid sequence of SEQ ID NO: 75 is substituted by a different amino acid residue.

In some embodiments, the VH region of the second antigen binding moiety includes three CDRs (e.g., HCDR1, HCDR2, and HCDR3) as identified in each of the VH sequences disclosed in the Sequence Listing. In some embodiments, the HCDR1 of the second antigen binding moiety includes the sequence of SEQ ID NO: 98. In some embodiments, the HCDR2 of the second antigen binding moiety includes the sequence of SEQ ID NO: 99. In some embodiments, the HCDR3 of the second antigen binding moiety includes the sequence of SEQ ID NO: 106 or SEQ ID NO: 100. In some embodiments, the HCDR1, HCDR2, and HCDR3 of the VH region of the first antigen binding moiety includes the sequences of SEQ ID NO: 98, SEQ ID NO: 99, and SEQ ID NO: 100, respectively. In some embodiments, the VH region of the second antigen binding moiety includes three HCDRs as identified in each of the VH sequences disclosed in the Sequence Listing, wherein one, two, three, four, or five of the amino acid residues in at least one of the HCDRs is substituted by a different amino acid residue. In some embodiments, the HCDR1 of the second antigen binding moiety includes the sequence of SEQ ID NO: 98, wherein one, two, three, four, or five of the amino acid residues in SEQ ID NO: 98 is substituted by a different amino acid residue. In some embodiments, the HCDR2 of the second antigen binding moiety includes the sequence of SEQ ID NO: 99, wherein one, two, three, four, or five of the amino acid residues in SEQ ID NO: 99 is substituted by a different amino acid residue. In some embodiments, the HCDR3 of the second antigen binding moiety includes the sequence of SEQ ID NO: 100, wherein one, two, three, four, or five of the amino acid residues in SEQ ID NO: 100 is substituted by a different amino acid residue.

In some embodiments, the second antigen binding moiety includes a VL region having at least 80% at least 90%, at least 95%, at least 96%, at least 97, at least 98%, or at least 99% sequence identity to a VL sequence identified in Table 4. In some embodiments, the second antigen binding moiety includes a VL region having at least 80%, at least 90%, at least 95%, at least 96%, at least 97, at least 98%, or at least 99% sequence identity to SEQ ID NO: 74. In some embodiments, the second antigen binding moiety includes a VL region having at least 80%, at least 90%, at least 95%, at least 96%, at least 97, at least 98%, or at least 99% sequence identity to SEQ ID NO: 76. In some embodiments, the second antigen binding moiety includes a VL region with an amino acid sequence having 100% sequence identity to a VL sequence identified in Table 4. In some embodiments, the second antigen binding moiety includes a VL region with an amino acid sequence having 100% sequence identity to SEQ ID NO: 74. In some embodiments, the second antigen binding moiety includes a VL region with an amino acid sequence having 100% sequence identity to SEQ ID NO: 76. In some embodiments, the second antigen binding moiety includes a VL region having an amino acid sequence corresponding to any one of the VL sequences identified in Table 4, wherein one, two, three, four, or five of the amino acid residues in the amino acid sequence is substituted by a different amino acid residue. In some embodiments, the second antigen binding moiety includes a VL region having an amino acid sequence corresponding to SEQ ID NO: 74, wherein one, two, three, four, or five of the amino acid residues in the amino acid sequence of SEQ ID NO: 74 is substituted by a different amino acid residue. In some embodiments, the second antigen binding moiety includes a VL region having an amino acid sequence corresponding to SEQ ID NO: 76, wherein one, two, three, four, or five of the amino acid residues in the amino acid sequence of SEQ ID NO: 76 is substituted by a different amino acid residue.

In some embodiments, the VL region of the second antigen binding moiety includes three CDRs (e.g., LCDR1, LCDR2, and LCDR3) as identified i in each of the VL sequences disclosed in the Sequence Listing. In some embodiments, the LCDR1 of the second antigen binding moiety includes the sequence of SEQ ID NO: 101. In some embodiments, the LCDR2 of the second antigen binding moiety includes the sequence of SEQ ID NO: 102. In some embodiments, the LCDR3 of the second antigen binding moiety includes the sequence of SEQ ID NO: 103. In some embodiments, the LCDR1, LCDR2, and LCDR3 of the VL region of the second antigen binding moiety includes the sequences of SEQ ID NO: 101, SEQ ID NO: 102, and SEQ ID NO: 103, respectively. In some embodiments, the VL region of the second antigen binding moiety includes three CDRs as identified in the Sequence Listing, wherein one, two, three, four, or five of the amino acid residues in at least one of the CDRs is substituted by a different amino acid residue. In some embodiments, the LCDR1 of the second antigen binding moiety includes the sequence of SEQ ID NO: 101, wherein one, two, three, four, or five of the amino acid residues in SEQ ID NO: 101 is substituted by a different amino acid residue. In some embodiments, the LCDR2 of the second antigen binding moiety includes the sequence of SEQ ID NO: 102, wherein one, two, three, four, or five of the amino acid residues in SEQ ID NO: 102 is substituted by a different amino acid residue. In some embodiments, the LCDR3 of the second antigen binding moiety includes the sequence of SEQ ID NO: 103, wherein one, two, three, four, or five of the amino acid residues in SEQ ID NO: 103 is substituted by a different amino acid residue.

Moieties of Interest

In some embodiments of the disclosure, the antibody or functional fragment thereof is conjugated or covalently bound to at least one moiety-of-interest (MOI) selected from the group consisting of therapeutic moieties, diagnostic agents, and moieties that improve pharmacokinetics. In some embodiments, the at least one MOI is selected from the group consisting of an anticancer agent, an anti-autoimmune disease agent, an anti-inflammatory agent, an anti-bacterial agent, an antimicrobial agent, an antibiotic, an anti-infectious disease agent, and an antiviral agent. In some embodiments, the at least one MOI is selected from the group consisting of cytotoxic anti-cancer agents, DNA chelators, microtubule inhibitors, topoisomerase inhibitors, translation initiation inhibitors, ribosome inactivating molecules, nuclear transport inhibitors, RNA splicing inhibitors, RNA polymerase inhibitors, and DNA polymerase inhibitors.

In some embodiments, the cytotoxic anti-cancer agent is selected from the group consisting of auristatins, dolastatins, tubulysins, maytansinoids, taxanes, vinca alkaloids, amatoxins, anthracyclines, calicheamycins, camptothecins, irinotecan, SN-38, combretastatins, duocarmycins, enediynes, epothilones, ethylenimines, mytomycins, pyrrolobenzodiazepines (PBDs), and calicheamicin.

In some embodiments, the at least one moiety-of-interest (MOI) is conjugated or covalently bound to a constant region of the engineered antibody or functional fragment thereof. In some embodiments, the at least one moiety-of-interest (MOI) is conjugated or covalently bound to a heavy chain constant (e.g., CH1, CH2, or CH3) region of the antibody or functional fragment thereof. In some embodiments, the at least one moiety-of-interest (MOI) is conjugated or covalently bound to a CH1 region of the antibody or functional fragment thereof. In some embodiments, the at least one moiety-of-interest (MOI) is conjugated or covalently bound to a light chain constant (CL) region of the antibody or functional fragment thereof. In principle, there are no particular restrictions to the number of MOI that can be conjugated or covalently bound to an engineered antibody of the disclosure. In some embodiments, the engineered antibody of the disclosure has an average number of MOIs per antibody (i.e., mean drug-to-antibody ratio, DAR) ranging to 1 to 20. In some embodiments, the engineered antibody of the disclosure has a mean number of MOIs per antibody ranging from about 1 to about 10. In some embodiments, the mean DAR is about 1 to about 5, about 2 to about 6, about 3 to about 7, about 3 to about 8, about 4 to about 9, about 5 to about 10, about 10 to about 15, about 15 to about 20, or about 10 to about 20.

One skilled in the art will appreciate that the complete amino acid sequence of an engineered antibody as disclosed herein can be used to construct a back-translated gene. For example, a DNA oligomer containing a nucleotide sequence coding for a given antibody can be synthesized. For example, several small oligonucleotides coding for portions of the desired antibody can be synthesized and then ligated. The individual oligonucleotides typically contain 5′ or 3′ overhangs for complementary assembly.

In addition to generating engineered antibodies via expression of nucleic acid molecules that have been altered by recombinant molecular biological techniques, a subject engineered antibody or functional fragment thereof in accordance with the present disclosure can be chemically synthesized. Chemically synthesized polypeptides are routinely generated by those of skill in the art.

Once assembled (by synthesis, site-directed mutagenesis or another method), the DNA sequences encoding an engineered antibody or functional fragment thereof as disclosed herein will be inserted into an expression vector and operably linked to an expression control sequence appropriate for expression of the engineered antibody or functional fragment thereof in the desired transformed host. Proper assembly can be confirmed by nucleotide sequencing, restriction mapping, and expression of a biologically active polypeptide in a suitable host. As is known in the art, in order to obtain high expression levels of a transfected gene in a host, the gene must be operably linked to transcriptional and translational expression control sequences that are functional in the chosen expression host.

The binding activity of the engineered antibodies or functional fragments thereof of the disclosure can be assayed by any suitable method known in the art. An antibody or polypeptide that “preferentially binds” or “specifically binds” (used interchangeably herein) to a target antigen or target epitope is a term well understood in the art, and methods to determine such specific or preferential binding are also known in the art. An antibody or polypeptide is said to exhibit “specific binding” or “preferential binding” if it reacts or associates more frequently, more rapidly, with greater duration and/or with greater affinity with a particular antigen or epitope than it does with alternative antigens or epitopes. An antibody or polypeptide “specifically binds” or “preferentially binds” to a target if it binds with greater affinity, avidity, more readily, and/or with greater duration than it binds to other substances. Also, an antibody or polypeptide “specifically binds” or “preferentially binds” to a target if it binds with greater affinity, avidity, more readily, and/or with greater duration to that target in a sample than it binds to other substances present in the sample. For example, an antibody or polypeptide that specifically or preferentially binds to an EphA2 epitope is an antibody or polypeptide that binds this epitope with greater affinity, avidity, more readily, and/or with greater duration than it binds to other EphA2 epitopes or non-EphA2 epitopes. It is also understood by reading this definition, for example, that an antibody or polypeptide (or moiety or epitope) which specifically or preferentially binds to a first target may or may not specifically or preferentially bind to a second target. As such, “specific binding” or “preferential binding” does not necessarily require (although it can include) exclusive binding.

A variety of assay formats may be used to select an antibody or polypeptide that specifically binds a molecule of interest. For example, solid-phase ELISA immunoassay, immunoprecipitation, Biacore™ (GE Healthcare, Piscataway, N.J.), KinExA, fluorescence-activated cell sorting (FACS), Octet™ (ForteBio, Inc., Menlo Park, Calif.) and Western blot analysis are among many assays that may be used to identify an antibody that specifically reacts with an antigen, or ligand binding portion thereof, that specifically binds with a cognate ligand or binding partner. Generally, a specific or selective reaction will be at least twice the background signal or noise, more typically more than 10 times background, even more typically, more than 50 times background, more typically, more than 100 times background, yet more typically, more than 500 times background, even more typically, more than 1000 times background, and even more typically, more than 10,000 times background. Also, in some embodiments, an antibody is said to “specifically bind” an antigen when the equilibrium dissociation constant (K_{D}) is <43 nM, <25 nM, <20 nM, <15 nM, <10 nM, or <7 nM.

The term “binding affinity” is herein used as a measure of the strength of a non-covalent interaction between two molecules, e.g., an antibody or portion thereof and an antigen. The term “binding affinity” is used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of engineered antibodies of the present disclosure towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g. by using a Biacore™ system, or KinExA.

### Nucleic Acid Molecules

In another aspect, provided herein are various recombinant nucleic acid molecules encoding the engineered antibodies of the disclosure, including expression cassettes, and expression vectors containing these nucleic acid molecules operably linked to heterologous nucleic acid sequences such as, for example, regulator sequences which allow expression of the engineered antibodies in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

Nucleic acid molecules of the present disclosure can be nucleic acid molecules of any length, including nucleic acid molecules that are generally between about 0.5 Kb and about 20 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb.

The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques or recombination of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

In some embodiments disclosed herein, the nucleic acid molecules of the disclosure include a nucleotide sequence encoding an engineered antibody having an amino acid sequence having at least 80%, 90%, 95%, 96%, 97, 98%, 99% sequence identity to the amino acid sequence of an engineered antibody as disclosed herein. In some embodiments, the nucleic acid molecules of the disclosure include a nucleotide sequence encoding an engineered antibody having an amino acid sequence having at least 80%, 90%, 95%, 96%, 97, 98%, 99% sequence identity to any one of the amino acid sequences identified in Table 4. In some embodiments, the nucleic acid molecules of the disclosure include a nucleotide sequence encoding an engineered antibody having an amino acid sequence having at least 80%, 90%, 95%, 96%, 97, 98%, 99% sequence identity to any one of the VH amino acid sequences identified in Table 3. In some embodiments, the nucleic acid molecules of the disclosure include a nucleotide sequence encoding an engineered antibody having an amino acid sequence having at least 80%, 90%, 95%, 96%, 97, 98%, 99% sequence identity to any one of the VL amino acid sequences identified in Table 4.

Some embodiments disclosed herein relate to vectors or expression cassettes including a recombinant nucleic acid molecule encoding the engineered antibodies as disclosed herein. As used herein, the term “expression cassette” refers to a construct of genetic material that contains coding sequences and enough regulatory information to direct proper transcription and/or translation of the coding sequences in a recipient cell, in vivo and/or ex vivo. The expression cassette may be inserted into a vector for targeting to a desired host cell and/or into a subject. As such, the term expression cassette may be used interchangeably with the term “expression construct”. As used herein, the term “construct” is intended to mean any recombinant nucleic acid molecule such as an expression cassette, plasmid, cosmid, virus, autonomously replicating polynucleotide molecule, phage, or linear or circular, single-stranded or double-stranded, DNA or RNA polynucleotide molecule, derived from any source, capable of genomic integration or autonomous replication, comprising a nucleic acid molecule where one or more nucleic acid sequences has been linked in a functionally operative manner, e.g. operably linked.

Also provided herein are vectors, plasmids or viruses containing one or more of the nucleic acid molecules encoding any of the engineered antibodies disclosed herein. The nucleic acid molecules described above can be contained within a vector that is capable of directing their expression in, for example, a cell that has been transformed/transduced with the vector. Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,”2nd Ed. (1989).

It should be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered. For example, vectors that can be used include those that allow the DNA encoding the engineered antibodies of the present disclosure to be amplified in copy number. Such amplifiable vectors are known in the art.

Accordingly, in some embodiments, the engineered antibodies as described herein, can be expressed from vectors, for example expression vectors. The vectors are useful for autonomous replication in a host cell or may be integrated into the genome of a host cell upon introduction into the host cell, and thereby are replicated along with the host genome (e.g., non-episomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are also included. Exemplary recombinant expression vectors can include one or more regulatory sequences, selected on the basis of the host cells to be used for expression, operably linked to the nucleic acid sequence to be expressed.

DNA vector can be introduced into prokaryotic or eukaryotic cells via conventional transformation or transfection techniques. Suitable methods for transforming or transfecting host cells can be found in Sambrook et al. (1989, supra) and other standard molecular biology laboratory manuals.

The nucleic acid sequences encoding the engineered antibodies of the disclosure, can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the engineered antibodies disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Vectors suitable for use include T7-based vectors for use in bacteria, the pMSXND expression vector for use in mammalian cells, and baculovirus-derived vectors for use in insect cells. In some embodiments, nucleic acid inserts, which encode the engineered antibody in such vectors, can be operably linked to a promoter, which is selected based on, for example, the cell type in which expression is sought.

In selecting an expression control sequence, a variety of factors should also be considered. These include, for example, the relative strength of the sequence, its controllability, and its compatibility with the actual DNA sequence encoding the subject polypeptide, particularly as regards potential secondary structures. Hosts should be selected by consideration of their compatibility with the chosen vector, the toxicity of the product coded for by the DNA sequences of this disclosure, their secretion characteristics, their ability to fold the polypeptides correctly, their fermentation or culture requirements, and the ease of purification of the products coded for by the DNA sequences.

Within these parameters one of skill in the art may select various vector/expression control sequence/host combinations that will express the desired DNA sequences on fermentation or in large scale animal culture, for example, using CHO cells or COS 7 cells.

The choice of expression control sequence and expression vector, in some embodiments, will depend upon the choice of host. A wide variety of expression host/vector combinations can be employed. Non-limiting examples of useful expression vectors for eukaryotic hosts, include, for example, vectors with expression control sequences from SV40, bovine papilloma virus, adenovirus and cytomegalovirus. Non-limiting examples of useful expression vectors for bacterial hosts include known bacterial plasmids, such as plasmids from E. coli, including col El, pCRI, pER32z, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Non-limiting examples of useful expression vectors for yeast cells include the 2p plasmid and derivatives thereof. Non-limiting examples of useful vectors for insect cells include pVL 941 and pFastBac™ 1.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). In some embodiments, the vector is a lentiviral vector, an adeno virus vector, an adeno-associated virus vector, or a retroviral vector. In some embodiments, the vector is a lentiviral vector.

Recombinant prokaryotic or eukaryotic cells that contain an engineered antibody or functional fragment thereof as disclosed herein, and/or contain and express a nucleic acid molecule that encodes any one of the engineered antibody or functional fragment thereof disclosed herein are also features of the disclosure. In some embodiments, a recombinant cell of the disclosure is a transfected cell, e.g, a cell into which a nucleic acid molecule, for example a nucleic acid molecule encoding an engineered antibody disclosed herein, has been introduced by means of recombinant methodologies and techniques. The progeny of such a cell are also considered within the scope of the disclosure. Cell cultures containing at least one recombinant cell as disclosed herein are also within the scope of the present disclosure. The terms, “cell”, “cell culture”, “cell line”, “recombinant cell”, “recipient cell” and “host cell” as used herein, include the primary subject cells and any progeny thereof, without regard to the number of transfers. It should be understood that not all progeny are exactly identical to the parental cell (due to deliberate or inadvertent mutations or differences in environment); however, such altered progeny are included in these terms, so long as the progeny retain the same functionality as that of the originally transformed cell.

The precise components of the expression system are not critical. For example, an engineered antibody as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed antibody can be purified from the expression system using routine biochemical procedures, and can be used, e.g., as therapeutic agents, as described herein.

In some embodiments, engineered antibodies obtained will be glycosylated or unglycosylated depending on the host organism used to produce the engineered antibodies. If bacteria are chosen as the host then the engineered antibodies produced will be unglycosylated. Eukaryotic cells, on the other hand, will glycosylate the engineered antibodies, although perhaps not in the same way as native polypeptides is glycosylated. The recombinant antibodies produced by the transformed host can be purified according to any suitable methods known in the art. Produced recombinant antibodies can be isolated from inclusion bodies generated in bacteria such as E. coli, or from conditioned medium from either mammalian or yeast cultures producing an engineered antibody of the disclosure using cation exchange, gel filtration, and or reverse phase liquid chromatography.

In addition or alternatively, another exemplary method of constructing a DNA sequence encoding the engineered antibodies of the disclosure is by chemical synthesis. This includes direct synthesis of a peptide by chemical means of the amino acid sequence encoding for an engineered antibody exhibiting the properties described. This method can incorporate both natural and unnatural amino acids at positions that affect the binding affinity of the engineered antibodies with a target protein. Alternatively, a gene which encodes the desired engineered antibodies can be synthesized by chemical means using an oligonucleotide synthesizer. Such oligonucleotides are designed based on the amino acid sequence of the desired engineered antibodies, and generally selecting those codons that are favored in the host cell in which the engineered antibody of the disclosure will be produced. In this regard, it is well recognized in the art that the genetic code is degenerate such that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated by those skilled in the art that for a given DNA sequence encoding a particular engineered antibody, there will be many DNA degenerate sequences that will code for that engineered antibody. For example, it will be appreciated that in addition to the DNA sequences for engineered antibodies provided herein, there will be many degenerate DNA sequences that code for the engineered antibodies disclosed herein. These degenerate DNA sequences are considered within the scope of this disclosure. Therefore, “degenerate variants thereof” in the context of this disclosure means all DNA sequences that code for and thereby enable expression of a particular engineered antibody.

The DNA sequence encoding the subject engineered antibody, whether prepared by site directed mutagenesis, chemical synthesis or other methods, can also include DNA sequences that encode a signal sequence. Such signal sequence, if present, should be one recognized by the cell chosen for expression of the engineered antibody. It can be prokaryotic, eukaryotic or a combination of the two. In general, the inclusion of a signal sequence depends on whether it is desired to secrete the engineered antibody as disclosed herein from the recombinant cells in which it is made. If the chosen cells are prokaryotic, the DNA sequence generally does not encode a signal sequence. If the chosen cells are eukaryotic, a signal sequence is generally included.

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g, either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of an engineered antibody) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary isolated nucleic acid molecules of the present disclosure can include fragments not found as such in the natural state. Thus, this disclosure encompasses recombinant molecules, such as those in which a nucleic acid sequence (for example, a sequence encoding an engineered antibody disclosed herein) is incorporated into a vector (e.g., a plasmid or viral vector) or into the genome of a heterologous cell (or the genome of a homologous cell, at a position other than the natural chromosomal location).

### Pharmaceutical Compositions

In some embodiments, the engineered antibodies, the nucleic acids, and/or the recombinant cells of the disclosure can be incorporated into compositions, including pharmaceutical compositions. Such compositions generally include the engineered antibodies, the nucleic acids, and/or the recombinant cells of the disclosure and a pharmaceutically acceptable carrier. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., anticancer agent) can also be incorporated into the compositions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, one or more isotonic agents, for example, sugars, polyalcohols such as mannitol, sorbitol, sodium chloride is included in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound (e.g., engineered antibodies, and/or nucleic acid molecules of the disclosure) can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches, and the like, can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, the subject engineered antibodies of the disclosure are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the subject engineered antibodies of the disclosure can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the engineered antibodies of the disclosure can also be prepared in the form of suppositories (e.g., with conventional suppository bases such as cocoa butter and other glycerides) or retention enemas for rectal delivery.

In some embodiments, the engineered antibodies of the disclosure can also be administered by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996).

In some embodiments, the subject engineered antibodies of the disclosure are prepared with carriers that will protect the engineered antibodies against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, the engineered antibodies of the disclosure can be further modified to prolong their half-life in vivo and/or ex vivo. Non-limiting examples of known strategies and methodologies suitable for modifying the engineered antibodies of the disclosure include (1) chemical modification of an engineered antibody described herein with highly soluble macromolecules such as polyethylene glycol (“PEG”) which prevents the engineered antibody from contacting with proteases; and (2) covalently linking or conjugating an engineered antibody described herein with a stable protein such as, for example, albumin. Accordingly, in some embodiments, the engineered antibodies of the disclosure can be fused to a stable protein, such as, albumin. For example, human albumin is known as one of the most effective proteins for enhancing the stability of polypeptides fused thereto and there are many such fusion proteins reported.

In some embodiments, the engineered antibodies of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the interferon. In some embodiments, the PEGylated or PASylated antibody contains a PEG or PAS moiety on only one amino acid. In other embodiments, the PEGylated or PASylated antibody contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The engineered antibodies may be coupled directly to PEG or PAS (e.g., without a linking group) through an amino group, a sulfhydryl group, a hydroxyl group, or a carboxyl group.

In some embodiments, the pharmaceutical compositions of the disclosure includes one or more pegylation reagent. As used herein, the term “PEGylation” means and refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the engineered antibodies of the disclosure using a variety of chemistries. In some embodiments, the pegylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the pegylation reagent is methoxy polyethylene glycol-succinimidyl propionate; for example said pegylation reagent is methoxy polyethylene glycol-succinimidyl propionate 5000 with an average molecular weight of 5,000 Daltons.

### Methods of the Disclosure

### Methods for Modulating Cellular Internalization and Modulating Cell-Type Selective Signaling

In various aspects of the disclosure, the engineered antibodies and functional fragments thereof as disclosed herein, nucleic acids encoding such engineered antibodies, and/or pharmaceutical compositions containing same can be used to modulate cellular internalization of cell-surface molecules. The term “modulating” refers to decreasing, reducing, inhibiting, increasing, inducing, activating, or otherwise affecting the cellular internalization of a cell-surface molecule.

In one aspect, some embodiments of the present disclosure relate to a method for modulating cellular internalization, including administering to a cell one or more of the following: (a) an engineered antibody or functional fragment thereof as disclosed herein; (b) a nucleic acid molecule as disclosed herein; and (c) a pharmaceutical composition as disclosed herein.

In another aspect, some embodiments of the present disclosure relate to a method for modulating cellular internalization, the method includes administering to a cell an engineered antibody or functional fragment thereof including: (a) a first antigen binding moiety capable of binding to a cell-surface guide antigen having a first rate of cellular internalization; and (b) a second antigen binding moiety capable of binding to a cell-surface effector antigen having a second rate of cellular internalization, wherein the internalization property of the engineered antibody or functional fragment thereof is determined by a relative surface density ratio of the guide antigen to the effector antigen, and wherein one of the two cellular internalization rates is at least 50%, at least 70%, at least 80%, or at least 90% greater than the other rate.

In yet another aspect, as discussed in greater detail below, some embodiments of the present disclosure relate to a method for treating a health condition or diseases (e.g., cancer) in a subject using an engineered antibody or a conjugate thereof as disclosed herein.

In accordance with the methods disclosed herein, it is possible to induce rapid internalization of a slowly internalizing antigen by operably linked an antigen binding moiety specific for such a slowly internalizing antigen with another antigen binding moiety specific for a rapidly internalizing antigen. In some embodiments, it is possible to induce slow internalization of a rapidly internalizing antigen by operably linking an antigen binding moiety specific for such rapidly internalizing antigen with another antigen binding moiety specific for a slowly internalizing antigen. In some embodiments of the methods described herein, the internalization property of the engineered antibody is converted from internalizing to non-internalizing. In some embodiments, the internalization property of the guide antigen and/or the effector antigen is converted from internalizing to non-internalizing. In some embodiments, the internalization property of an internalizing antigen (e.g., guide antigen or effector antigen) is converted from internalizing to non-internalizing by using an engineered antibody as disclosed herein which includes an antigen binding moiety specific for such an internalizing antigen operably linked with another antigen binding moiety specific for a non-internalizing antigen. For example, in some embodiments of the disclosed methods, the internalization property of an internalizing guide antigen is converted from internalizing to non-internalizing by using an engineered antibody as disclosed herein which includes an antigen binding moiety specific for the internalizing guide antigen operably linked with another antigen binding moiety specific for a non-internalizing effector antigen. In some embodiments, the internalization property of an internalizing effector antigen is converted from internalizing to non-internalizing by using an engineered antibody as disclosed herein which includes an antigen binding moiety specific for the internalizing effector antigen operably linked with another antigen binding moiety specific for a non-internalizing guide antigen. In some embodiments of the disclosed methods, the internalization property of the engineered antibody is converted from non-internalizing to internalizing. In some embodiments, the internalization property of a non-internalizing antigen (e.g., guide antigen or effector antigen) is converted from non-internalizing to internalizing. In some other embodiments, the internalization property of a non-internalizing guide antigen is converted from non-internalizing to internalizing by using an engineered antibody as disclosed herein which includes an antigen binding moiety specific for such non-internalizing guide antigen operably linked with another antigen binding moiety specific for an internalizing effector antigen. In some other embodiments, the internalization property of a non-internalizing effector antigen is converted from non-internalizing to internalizing by using an engineered antibody as disclosed herein which includes an antigen binding moiety specific for such non-internalizing effector antigen operably linked with another antigen binding moiety specific for an internalizing guide antigen.

In some embodiments, the guide antigen has a rate of cellular internalization that is greater than the cellular internalization rate of the effector antigen, in which case the guide antigen is a rapidly internalizing antigen and the effector antigen is a slowly internalizing antigen. In some embodiments, the guide antigen has a rate of cellular internalization of at least about 50% greater than the cellular internalization rate of the effector antigen. In some embodiments, the guide antigen has a rate of cellular internalization of at least about 50%, 60%, 70%, 80%, or 90% greater than the cellular internalization rate of the effector antigen. In some embodiments, the engineered antibody of the disclosure increases the internalization rate of the slowly internalizing antigen (e.g., effector antigen) by at least 10%, 20%, 30%, 40%, 50%, 60%, 70%, 80%, 90%, 99% or 99.9% as compared to a control (e.g., a monospecific antibody comprising only the slowly internalizing antigen) by operably linked an antigen binding moiety specific for the slowly internalizing antigen with another antigen binding moiety specific for a rapidly internalizing antigen (e.g. guide antigen). In some embodiments, the engineered antibody of the disclosure reduces the internalization rate of the rapidly internalizing antigen (e.g., guide antigen) by at least 10%, 20%, 30%, 40%, 50%, 60%, 70%, 80%, 90%, 99% or 99.9% as compared to a control (e.g., a monospecific antibody comprising only the rapidly internalizing antigen) by operably linking an antigen binding moiety specific for the rapidly internalizing antigen with another antigen binding moiety specific for a slowly internalizing antigen (e.g., effector antigen).

In some embodiments, the effector antigen has a rate of cellular internalization that is greater than the cellular internalization rate of the guide antigen, in which case the effector antigen is a rapidly internalizing antigen and the guide antigen is a slowly internalizing antigen. In some embodiments, the effector antigen has a rate of cellular internalization of at least about 50% greater than the cellular internalization rate of the guide antigen. In some embodiments, the effector antigen has a rate of cellular internalization of at least about 50%, 60%, 70%, 80%, or 90% greater than the cellular internalization rate of the guide antigen. In some embodiments, the engineered antibody of the disclosure increases the internalization rate of the slowly internalizing antigen (e.g., guide antigen) by at least 10%, 20%, 30%, 40%, 50%, 60%, 70%, 80%, 90%, 99% or 99.9% as compared to a control (e.g., a monospecific antibody comprising only the slowly internalizing antigen) by operably linked an antigen binding moiety specific for the slowly internalizing antigen with another antigen binding moiety specific for a rapidly internalizing antigen (e.g. effector antigen). In some embodiments, the engineered antibody of the disclosure reduces the internalization rate of the rapidly internalizing antigen (e.g., effector antigen) by at least 10%, 20%, 30%, 40%, 50%, 60%, 70%, 80%, 90%, 99% or 99.9% as compared to a control (e.g., a monospecific antibody comprising only the rapidly internalizing antigen) by operably linking an antigen binding moiety specific for the rapidly internalizing antigen with another antigen binding moiety specific for a slowly internalizing antigen (e.g., guide antigen).

In some embodiments, the cell-surface guide antigen is an internalizing cell surface antigen. In some embodiments, the cell-surface effector antigen is a non-internalizing cell surface antigen. In some embodiments, the relative surface density ratio of the guide antigen to the effector antigen is greater than a threshold value. In some embodiments, the threshold value is about 1:1, about 1:2, about 1:3, about 1:4, about 1:5, about 1:10, about 1:20, or about 1:30. Accordingly, in some embodiments, the relative surface density ratio of the guide antigen to the effector antigen is greater than about 1:1, about 1:2, about 1:3, about 1:4, about 1:5, about 1:10, about 1:20, or about 1:30. In some particular embodiments, the relative surface density ratio of the guide antigen to the effector antigen is greater than about 1:5. In some embodiments, the relative surface density ratio of the guide antigen to the effector antigen is lower than a threshold value. In some embodiments, the threshold value is about 1:1, about 1:2, about 1:3, about 1:4, about 1:5, about 1:10, about 1:20, or about 1:30. Accordingly, in some embodiments, the relative surface density ratio of the guide antigen to the effector antigen is lower than about 1:1, about 1:2, about 1:3, about 1:4, about 1:5, about 1:10, about 1:20, or about 1:30. In some particular embodiments, the relative surface density ratio of the guide antigen to the effector antigen is lower than about 1:5.

In some other embodiments, the relative surface density ratio of the effector antigen to the guide antigen is greater than a threshold value. In some embodiments, the threshold value is about 1:1, about 1:2, about 1:3, about 1:4, about 1:5, about 1:10, about 1:20, or about 1:30. Accordingly, in some embodiments, the relative surface density ratio of the effector antigen to the guide antigen is greater than about 1:1, about 1:2, about 1:3, about 1:4, about 1:5, about 1:10, about 1:20, or about 1:30.

In some embodiments, the methods of the disclosure further include modulating cell surface density of the guide antigen and/or cell surface density of the effector antigen. One skilled in the art will readily appreciate that it is possible to modulate guide-to-effector ratio by using techniques known in the art for modulating expression and/or function of a target gene or target protein. Non-limiting examples of such techniques include gene suppression, small RNA interference, partial gene knock-out, small molecules or protein/peptide with signaling functions that alter cell metabolism, proliferation, migration, death, senescence, differentiation and immune regulation.

In yet another aspect, some embodiments of the present disclosure relate to a method for modulating cell-type selective signaling in a subject, the method includes administering to a cell an engineered antibody or functional fragment thereof including: (a) a first antigen binding moiety capable of binding to a cell-surface guide antigen wherein the guide antigen is expressed in the subject in a cell-type selective manner and has a first rate of cellular internalization; and (b) a second antigen binding moiety capable of binding to a cell-surface effector antigen having a second rate of cellular internalization, wherein the internalization property of the engineered antibody or functional fragment thereof is determined by a relative surface density ratio of the guide antigen to the effector antigen, and wherein one of the two cellular internalization rates is at least 50%, at least 70%, at least 80%, or at least 90% greater than the other rate. In some embodiments, an engineered antibody described herein modulates a signaling pathway, which can be upregulation or downregulation of such signaling pathway. In some embodiments, an engineered antibody of the disclosure can function as an agonist and upregulates (enhances, stimulates, promotes, activates or increases) a signaling pathway of interest, i.e. a target pathway. In some embodiments, an engineered antibody described herein increases the activity of the target pathway by at least 10%, 20%, 30%, 40%, 50%, 60%, 70%, 80%, 90%, 99% or 99.9% as compared to a control (e.g., no antibody or a monospecific antibody). In some other embodiments, upregulation of a target signaling pathway includes turning on or initiating the pathway that was off or substantially not active. In another example, an engineered antibody described herein can function as an antagonist and downregulates (suppresses, inhibits, reduces, decreases or diminishes) the target pathway. In some embodiments, an engineered antibody disclosed herein decreases the activity of the target pathway by at least 10%, 20%, 30%, 40%, 50%, 60%, 70%, 80%, 90%, 99% or 99.9% as compared to a control (e.g., no antibody or a monospecific antibody). In some embodiments, downregulation of a target signaling pathway includes turning off or substantially blocking the pathway that was on or substantially activity.

### Methods of Treatment

As discussed above, experimental results presented herein demonstrate that the guide-effector bispecific antibody designs disclosed herein can be used in development of new tools for manipulation of the internalizing property of a cell surface antigen. In particular, experimental results presented herein illustrate that the internalizing propensity of a given cell surface antigen can be manipulated is significantly impacted by its neighboring antigen(s), and can be readily manipulated in either direction through bispecific-based targeting of appropriately selected guide/effector pairs, and this phenomenon can be exploited for therapeutic targeting.

Some embodiments of the present disclosure relate to a method for treating a health condition or diseases (e.g., cancer) in a subject using an engineered antibody or a conjugate thereof as disclosed herein. In some embodiments, the methods include administering to a subject in need thereof a therapeutically effective amount of an engineered antibody disclosed herein, a conjugate thereof, or a pharmaceutical composition comprising the same, alone (e.g., as a monotherapy) or in combination (e.g., as a combination therapy) with one or more additional agents, e.g. a pharmaceutically acceptable excipient. In certain aspects, an engineered antibody or a pharmaceutical composition that is administered to a subject specifically targets a cell wherein a signaling pathway is modulated as a result of the treatment.

In one aspect, some embodiments of the disclosure relate to a method for treating a health condition or disease in a subject in need thereof, the method includes administering to the subject a therapeutically effective amount of an engineered antibody or functional fragment thereof including: (a) a first antigen binding moiety capable of binding to a cell-surface guide antigen having a first rate of cellular internalization; and (b) a second antigen binding moiety capable of binding to a cell-surface effector antigen having a second rate of cellular internalization, wherein the internalization property of the engineered antibody or functional fragment thereof is determined by a relative surface density ratio of the guide antigen to the effector antigen; and wherein one of the two cellular internalization rates is at least 50%, at least 70%, at least 80%, or at least 90% greater than the other rate.

In another aspect, some embodiments of the present disclosure relate to a method for killing a cancer cell, the method includes administering to said cell an engineered antibody or functional fragment thereof as disclosed herein. In some embodiments, the engineered antibody or functional fragment thereof includes: (a) a first antigen binding moiety capable of binding to a cell-surface guide antigen having a first rate of cellular internalization; and (b) a second antigen binding moiety capable of binding to a cell-surface effector antigen having a second rate of cellular internalization.

In yet another aspect, some embodiments of the present disclosure relate to a method for killing a tumor cell, the method includes administering to said tumor cell an engineered antibody or functional fragment thereof as disclosed herein. In some embodiments of the disclosed methods, the engineered antibody or functional fragment thereof includes a first antigen binding moiety capable of binding to an ephrin receptor A2 (EphA2) expressed on the surface of said tumor cell; and a second antigen binding moiety capable of binding to an ALCAM expressed on the surface of the same tumor cell. In some embodiments, the surface density ratio of EphA2 to ALCAM is greater than a threshold value of about 1:5.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The efficacy of treatment can be determined by a skilled clinician. However, one skilled in the art will appreciate that a treatment is considered effective if any one or all of the signs or symptoms or markers of disease are improved or ameliorated. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount of a composition as disclosed herein includes an amount sufficient to promote a particular beneficial effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments, the health condition or disease is a cancer. In some embodiments, the engineered antibodies, conjugates thereof, and functional fragments thereof as disclosed herein, nucleic acids encoding such engineered antibodies, and/or pharmaceutical compositions containing same are administered to an individual (e.g. a human patient) to, for example, reduce the viability and/or invasiveness of cancerous cells, e.g., to reduce tumor size or metastasis, reduce tumor load, and/or improve the clinical outcome in patients. In certain aspects, antibody compositions can be used to disrupt the cell cycle of the cancer cell, and facilitate entry of the cell into apoptosis, e.g., by inducing cancerous cells to enter the pre-GO cell cycle phase. The methods relating to cancer contemplated herein include, for example, use of antibody therapy alone or in combination with anti-cancer vaccine or therapy, as well as use of antibodies generated using an effector and/or guide antigens in anti-cancer vaccines (e.g., by passive immunization) or therapies. The methods are useful in the context of treating or preventing a wide variety of cancers. In one aspect, cancer refers to a general term encompassing primary cancer and metastatic cancer. In some embodiments, primary cancer may be meant a group of tumor cells, which have acquired at least one characteristic feature of cancer cells, however have not yet invaded the neighboring tissues and hold together in a tumor localized at the place of primary origin. In some other embodiments, metastatic cancer may be meant a group of tumor cells, which originate from the cells of a primary cancer, which have invaded the tissue surrounding said primary cancer, disseminated through the body, adhered at a new distant place and grown to a new tumor. Examples of cancers include, but are not limited to, pancreatic cancers, colon cancers, ovarian cancers, prostate cancers, lung cancers, mesothelioma, breast cancers, urothelial cancers, liver cancers, head and neck cancers, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the engineered antibodies, conjugates thereof, and functional fragments thereof as disclosed herein, nucleic acids encoding such engineered antibodies, and/or pharmaceutical compositions containing same are used in an anti-cancer therapy, where the cancerous cells present a cell-specific marker, which can serve as a guide antigen for a bispecific antibody of the present disclosure on an extracellularly accessible cell surface. Cancers particularly amenable to therapy using bispecific antibody of the present disclosure include those targeted by the antibody through biding to the guide antigen. In some embodiments, the presence or expression level of such a guide antigen in normal human tissue or cells can be transient and low abundance as compared to cancer cells that overexpress the guide antigen. The guide antigen can be prevalent primarily in abnormal cells, such as cancer cells. Since expression of high levels of the guide antigen may exist predominantly in cancer cells, treatment with bispecific antibody of the present disclosure or the composition comprising the antibody can be used to treat the cancer cells with high specificity or selectivity, minimizing non-specific, cytotoxicity to non-cancerous or healthy cells.

In some embodiments, a mode of treatment is to modulate a signaling pathway using an engineered antibody of the present disclosure. Dysregulation of a signaling pathway is often associated with occurrence and/or advancement of a disease or condition in that modulation of such signaling pathway can result in effective treatment of the disease or condition. In some examples, a disease or condition may be related to dysregulation of one or more signaling pathway and such dysregulation may be ameliorated or diminished by modulation of another signaling pathway. In such an occasion, up- or downregulation of a signaling pathway using the engineered antibody of the present disclosure that can counteract or reduce the activity of the dysregulated signaling pathway can provide an effective treatment means.

In some embodiments, the cells subjected to the treatment using an engineered antibody of the present disclosure or a composition comprising the antibody are not limited to cancer cells but encompass any cells where a cellular internalization and signaling modulation may be desired. Such cells include, but not limited, to immune effector cells such as natural killer cell(s), T cell(s), dendritic cell(s) and macrophage(s).

### Dosage

Dosage, toxicity and therapeutic efficacy of the engineered antibodies of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. For example, compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

In the methods of the present disclosure, an effective amount of an engineered antibody of the present disclosure or the composition comprising the antibody is administered to an individual in need thereof. For example, in some embodiments, the engineered antibody inhibits growth, metastasis and/or invasiveness of a cancer cell(s) in an individual when the antibody or the composition thereof is administered in an effective amount. The amount administered varies depending upon the goal of the administration, the health and physical condition of the individual to be treated, age, the taxonomic group of individual to be treated (e.g., human, non-human primate, primate, etc.), the degree of resolution desired, the formulation of the engineered antibody or composition, the treating clinician's assessment of the medical situation, and other relevant factors. It is expected that the amount will fall in a relatively broad range that can be determined through routine trials. For example, the amount of the engineered antibody or composition thereof employed to inhibit cancer cell growth, metastasis and/or invasiveness is not more than about the amount that could otherwise be irreversibly toxic to the subject (i.e., maximum tolerated dose). In other cases the amount is around or even well below the toxic threshold, but still in an immunoeffective concentration range, or even as low as threshold dose.

Individual doses are generally not less than an amount required to produce a measurable effect on the individual, and may be determined based on the pharmacokinetics and pharmacology for absorption, distribution, metabolism, and excretion (“ADME”) of the antibody, and thus based on the disposition of the composition within the individual. This includes consideration of the route of administration as well as dosage amount, which can be adjusted for, e.g., parenteral (applied by routes other than the digestive tract for systemic or local effects) applications. For instance, administration of an engineered antibody or composition thereof is generally via injection and often intravenous, intramuscular, intratumoral, or a combination thereof.

An engineered antibody or composition thereof may be administered by infusion or by local injection, e.g. by infusion at a rate of about 10 mg/h to about 200 mg/h, about 50 mg/h to about 400 mg/h, including about 75 mg/h to about 375 mg/h, about 100 mg/h to about 350 mg/h, about 150 mg/h to about 350 mg/h, about 200 mg/h to about 300 mg/h, about 225 mg/h to about 275 mg/h. Exemplary rates of infusion can achieve a desired therapeutic dose of, for example, about 0.5 mg/m²/day to about 10 mg/m²/day, including about 1 mg/m²/day to about 9 mg/m2/day, about 2 mg/m²/day to about 8 mg/m²/day, about 3 mg/m²/day to about 7 mg/m²/day, about 4 mg/m²/day to about 6 mg/m²/day, about 4.5 mg/m²/day to about 5.5 mg/m²/day. Administration (e.g., by infusion) can be repeated over a desired period, e.g., repeated over a period of about 1 day to about 5 days or once every several days, for example, about five days, over about 1 month, about 2 months, etc. It also can be administered prior, at the time of, or after other therapeutic interventions, such as surgical intervention to remove cancerous cells. An engineered antibody or composition thereof can also be administered as part of a combination therapy, in which at least one of an immunotherapy, a cancer chemotherapy or a radiation therapy is administered to the subject.

### Routes of Administration

In some embodiments of the disclosure, the engineered antibodies, conjugates thereof, and functional fragments thereof as disclosed herein, nucleic acids encoding such engineered antibodies, and/or pharmaceutical compositions containing same can be formulated to be compatible with its intended route of administration. For example, the engineered antibodies, conjugates thereof, and functional fragments thereof as disclosed herein, nucleic acids encoding such engineered antibodies, and/or pharmaceutical compositions containing same may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

In practicing the methods, routes of administration (path by which the engineered antibodies, conjugates thereof, and functional fragments thereof as disclosed herein, nucleic acids encoding such engineered antibodies, and/or pharmaceutical compositions containing same are brought into an individual or a subject) may vary. An engineered antibody or composition thereof can be administered systemically (e.g., by parenteral administration, e.g., by an intravenous route) or locally (e.g., at a local tumor site, e.g., by intratumoral administration (e.g., into a solid tumor, into an involved lymph node in a lymphoma or leukemia), administration into a blood vessel supplying a solid tumor, etc.).

In some embodiments, an engineered antibody described herein is formulated for parenteral administration. In some cases, the engineered antibody is formulated for intravenous, subcutaneous, intramuscular, intra-arterial, intracranial, intracerebral, intracerebroventricular or intrathecal administration. In some instances, the engineered antibody is administered to a subject as an injection. In other instances, the engineered antibody is administered to a subject as an infusion.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The term “unit dosage form,” as used herein, refers to physically discrete units suitable as unitary dosages for human and animal subjects, each unit containing a predetermined quantity of compounds of the present disclosure calculated in an amount sufficient to produce the desired effect in association with a pharmaceutically acceptable diluent, carrier or vehicle. The specifications for the novel unit dosage forms depend on the particular compound employed and the effect to be achieved, and the pharmacodynamics associated with each compound in the individual as well as the target disease or condition and the stage thereof in the individual.

### Systems and Kits

Also provided herein are systems and kits including the engineered antibodies, recombinant nucleic acids, recombinant cells, or pharmaceutical compositions provided and described herein as well as written instructions for making and using the same. For example, provided herein, in some embodiments, are systems and/or kits that include one or more of: an engineered antibody as described herein, a recombinant nucleic acid molecule as described herein, a recombinant cell as described herein, or a pharmaceutical composition as described herein. In some embodiments, the systems and/or kits of the disclosure further include one or more syringes (including pre-filled syringes) and/or catheters (including pre-filled syringes) used to administer one any of the provided engineered antibodies, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating cellular internalization, modulating cell-type selective signaling in a subject, or treating a disease in a subject in need thereof.

Any of the above-described systems and kits can further include one or more additional reagents, where such additional reagents can be selected from: dilution buffers; reconstitution solutions, wash buffers, control reagents, control expression vectors, negative control antibodies, positive control antibodies, reagents for in vitro production of the engineered antibodies.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

Additional embodiments are disclosed in further detail in the following examples, which are provided by way of illustration and are not in any way intended to limit the scope of this disclosure or the claims.

### Example 1

### Identification of a High-Affinity ALCAM Antibody and Generation of the ALCAMxEphA2 Bispecific Antibodies

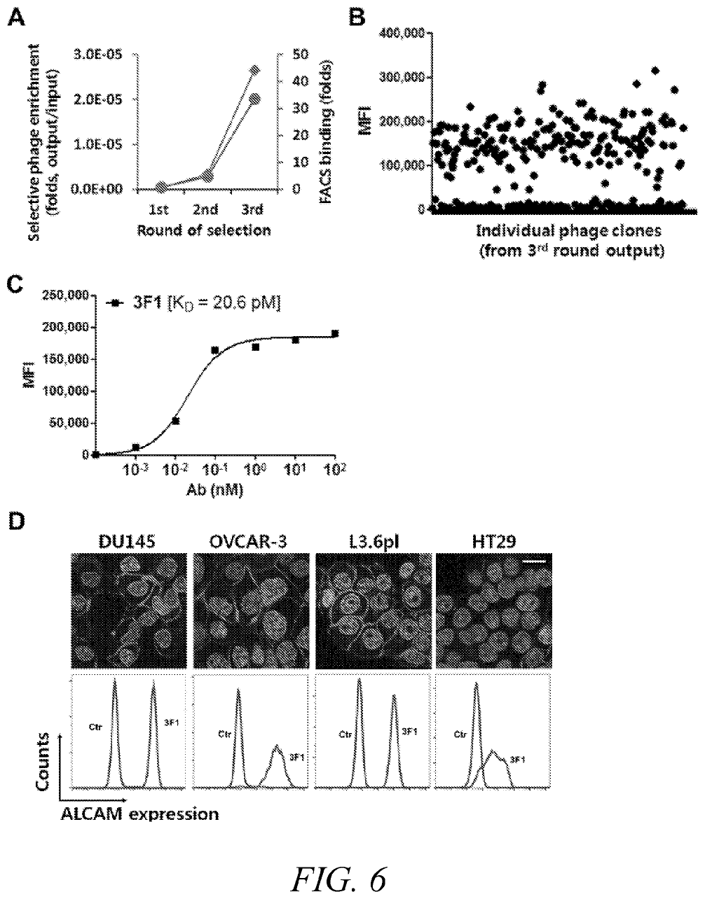

To identify human antibodies against ALCAM, scFv phage display library selection was performed against N-terminal Ig-like V1-V2 domain of ALCAM (FIG. 6A). A panel of binding phage by FACS screening on the ALCAM^{High} DU145 prostate cancer cell line was identified (FIG. 6B), and further identified an antibody 3F1 that binds with high affinity as an IgG1 (apparent K_{D}=20.6 pM) to DU145 cells (FIG. 6C). Internalization of the 3F1 IgG on a panel of tumor cell lines by confocal microscopy was studied and it was found that this antibody is non- or slowly internalizing (FIG. 6D).

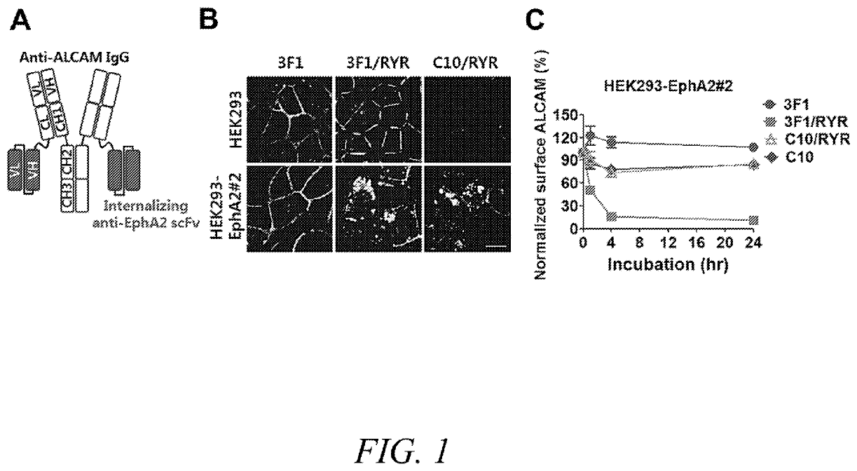

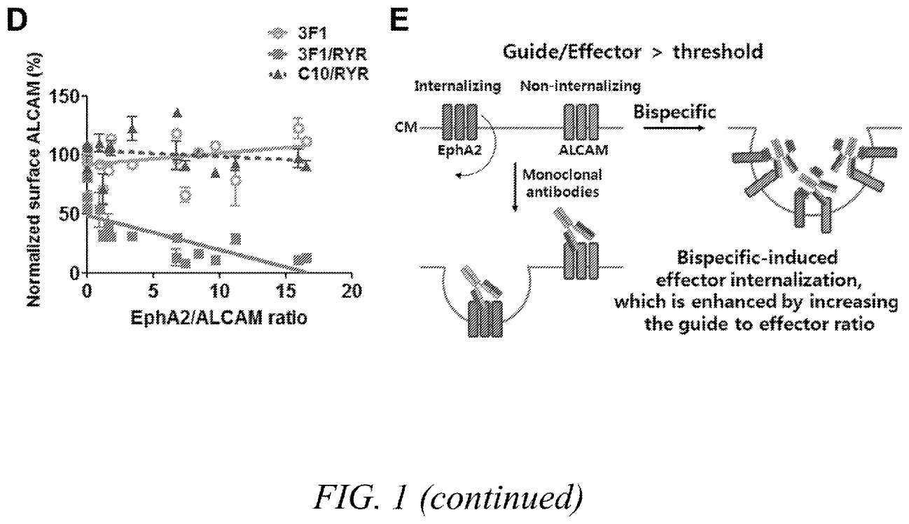

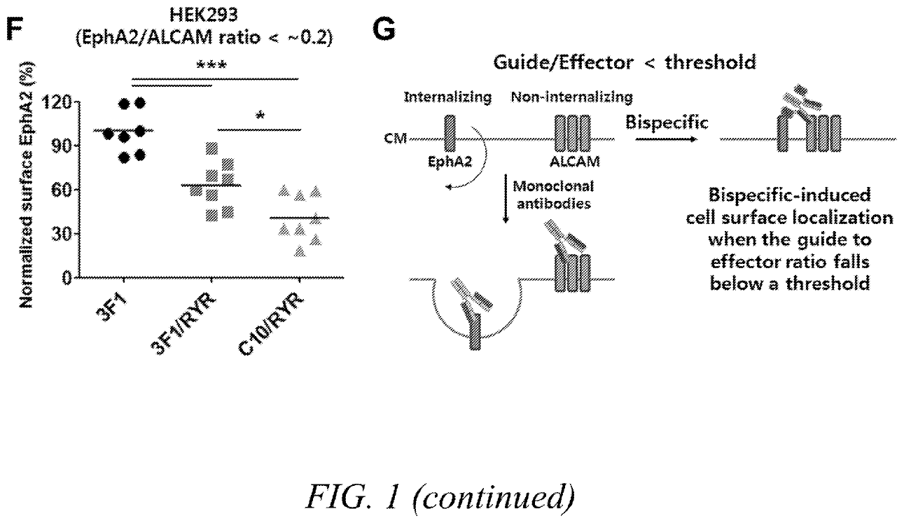

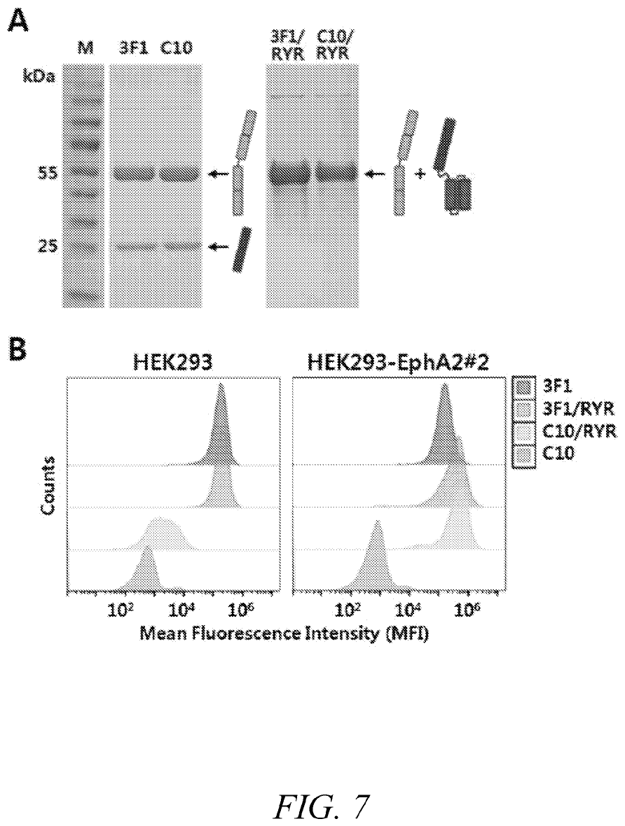

A tetravalent bispecific IgG-scFv (bsIgG) was constructed and composed of the non-internalizing anti-ALCAM 3F1 IgG backbone and an internalizing anti-EphA2 scFv (RYR) fused to the C terminus of the 3F1 light chain (FIG. 1A). The anti-EphA2 scFv (RYR) was identified from a previous study where high-content analysis was used to identify macropinocytosing antibodies. For control, a non-binding C10 IgG was used to construct the control C10/RYR bsIgG (binding to EphA2 only). SDS-PAGE analysis showed the expected electrophoresis pattern of monoclonal and bispecific antibodies (FIG. 7A). The binding specificity of bsIgGs was next studied using the HEK293 cell line that expresses ALCAM, and an engineered HEK293 cell line that stably expresses a high level of EphA2 (HEK293-EphA2 #2). As shown in FIG. 7B, the anti-ALCAM 3F1 IgG bound to both HEK293 and HEK293-EphA2 #2 cells as expected. The 3F1/RYR bsIgG bound at a higher level to HEK293-EphA2 #2 (ALCAM^{high}EphA2^{high}) compared with HEK293 (ALCAM^{high}EphA21^{high}). The control C10/RYR bsIgG that binds to EphA2 only showed specific binding to HEK293-EphA2 #2 but not HEK293 cells. Using these two cell line models, internalization activity of the 3F1/RYR bsIgG was studied by confocal microscopy. As shown in FIG. 1B, the 3F1/RYR bsIgG acquired effective internalization capacity in an EphA2-dependent manner—it is internalized by the HEK293-EphA2 #2 but not the HEK293 cell line. The control C10/RYR bsIgG is internalized by HEK293-EphA2 #2 but not HEK293. The result shows that in the guide-effector bispecific design described herein, the internalizing arm (EphA2, the guide) can impart the non-internalizing arm (ALCAM, the effector) and the bispecific as a whole with internalizing properties.

### Example 2

### The Non-Internalizing Antigen can be Rendered Internalizing by the Bispecific in a Time and Guide to Effector Ratio-Dependent Manner

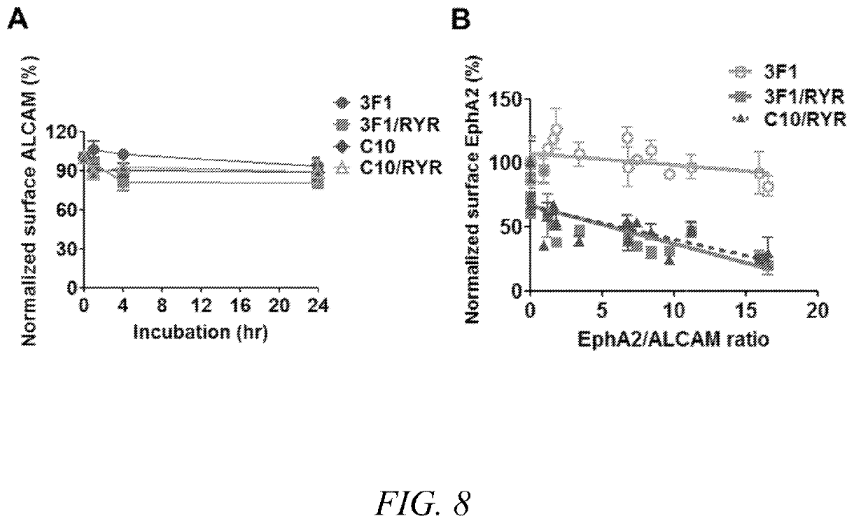

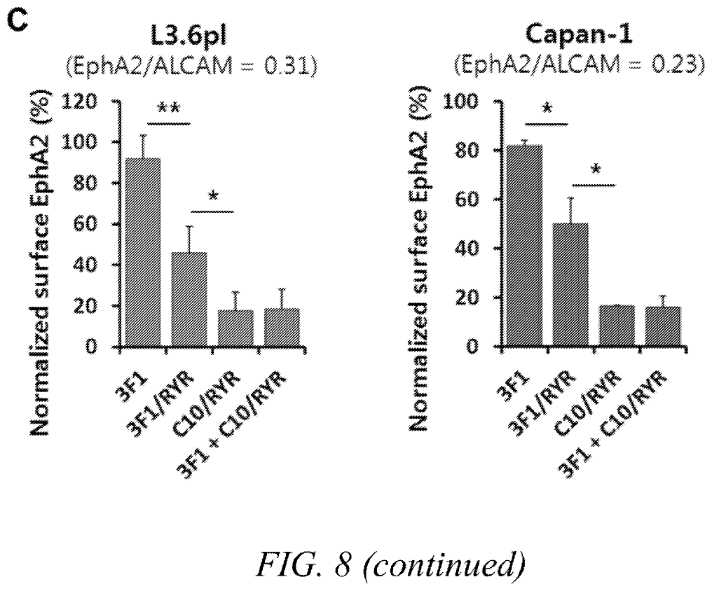

To quantitatively investigate cell surface antigen removal by antibody-induced antigen internalization, quantitative FACS analysis was conducted to measure ALCAM and EphA2 copy numbers on the cell surface (herein referred to as antigen density). As shown in FIG. 1C, the ALCAM level on HEK293-EphA2 #2 cells incubated with the bispecific 3F1/RYR was decreased by ˜90% within the first 4 hours of incubation. There was no significant change of the surface ALCAM level after treatment with monoclonal anti-ALCAM 3F1, the control monoclonal C10, or the control bispecific C10/RYR (FIG. 1C). Efficient ALCAM removal from cell surface by 3F1/RYR was only observed in HEK293-EphA2 #2 (ALCAM^{high}EphA2^{high}) but not HEK293 (ALCAM^{high}EphA2^{low}) (FIG. 8A). It was further sought to determine whether the antigen removal efficiency is influenced by the guide-to-effector ratio (EphA2/ALCAM). To generate HEK293-based cell models with varying EphA2/ALCAM ratios, EphA2 and/or ALCAM levels were manipulated by three ways: 1) transient transfection of EphA2-expressing plasmid, 2) transient co-transfection of EphA2-expressing plasmid and ALCAM-siRNA, and 3) lentiviral transduction of the EphA2 gene to achieve stable EphA2 expression. These cells showed varying EphA2/ALCAM ratios and varying patterns of surface antigen removal following the bispecific 3F1/RYR treatment. For example, the monoclonal anti-ALCAM 3F1 IgG or the control C10/RYR bsIgG did not remove ALCAM from the cell surface, whereas the 3F1/RYR bsIgG efficiently removed surface ALCAM (FIG. 1D). By Pearson's correlation coefficient analysis, the effect significantly increases as the EphA2/ALCAM ratio increases (FIG. 1D). With regard to EphA2, the anti-ALCAM 3F1 IgG did not reduce surface EphA2 as expected, but the 3F1/RYR and the control C10/RYR that binds to EphA2 removed EphA2 efficiently from the cell surface (FIG. 8B). The ability of the bispecific 3F1/RYR to remove surface ALCAM is affected by the ratio of EphA2 to ALCAM (guide to effector antigen ratio, outlined in FIG. 1E). As summarized in Table 1, when the ratio is <1:5 (0.2), only a small fraction of ALCAM is removed (20-35%). When the ratio is between 0.9-3.5, 45-65% surface ALCAM is removed. When the ratio is >3.5, greater than 70% of surface ALCAM is removed.

### Example 3

### Internalization and Non-Internalization are Interconvertible Properties in a Bispecific Design

While it has been shown that a non-internalizing antigen (ALCAM) can be induced to internalize by the anti-EphA2/ALCAM bispecific when the EphA2 to ALCAM ratio is above a threshold, this experiments investigates if the rapidly internalizing EphA2 can be rendered slowly or non-internalizing by the presence of ALCAM at certain EphA2 to ALCAM ratio. Using HEK293 cell line (ALCAM^{high}EphA21^{low}) as the model, it was found that when the EphA2 to ALCAM ratio was <0.2, EphA2 internalization was greatly retarded, resulting in a higher fraction of surface bound EphA2 when targeted by the 3F1/RYR bsIgG but not the control C10/RYR (FIG. 1F), suggesting that internalization and non-internalization are interconvertible properties and the relative abundance of internalizing vs. non-internalizing antigen profoundly impacts cell surface antigen turn over when targeted by bispecific antibodies (as outlined in FIG. 1G).

TABLE 1
Summary of antigen density, guide to effector ratio, and
ALCAM turnover efficiency on various cell line models.

| | | Antigen density and ratio | | | | | |
|---|---|---|---|---|---|---|---|
| | | (Mean ± SD, n = 2) | | | ALCAM Internalization | | |
| | | | | EphA2/ | (% over ctrl, Mean, n = 2) | | |
| Cell models | | EphA2 | ALCAM | ALCAM | 3F1 | 3F1/RYR | C10/RYR |
| HEK293 | | 5,064 ± | 335,719 ± | 0.02 | 6.6 | 19.6 | 10.8 |
| | | 858 | 2,475 | | | | |
| Transiently | HEK293- | 357,994 ± | 286,531 ± | 1.25 | 29.4 | 68.9 | 28.6 |
| EphA2- | EphA2-0.02 | 83,557 | 6,581 | | | | |
| transfected | HEK293- | 1,081,566 ± | 325,339 ± | 3.32 | 7.7 | 68.9 | −22.4 |
| HEK293 | EphA2-0.1 | 89,489 | 17,985 | | | | |
| | HEK293- | 2,130,189 ± | 311,259 ± | 6.84 | 3.5 | 70.1 | −36.3 |
| | EphA2-0.5 | 210,485 | 42,142 | | | | |
| | HEK293- | 3,386,996 ± | 301,854 ± | 11.22 | 21.3 | 71.2 | 7.1 |
| | EphA2-2.5 | 236,937 | 3,913 | | | | |
| Stably | HEK293- | 18,398 ± | 320,850 ± | 0.06 | 2.8 | 20.7 | −9.6 |
| EphA2- | EphA2#C9 | 1,606 | 36,242 | | | | |
| expressing | HEK293- | 23,508 ± | 202,617 ± | 0.12 | 3.7 | 16.5 | −16.5 |
| HEK293 | EphA2#42 | 2,955 | 11,141 | | | | |
| | HEK293- | 1,876,422 ± | 279,389 ± | 6.72 | −17.9 | 87.2 | 0.5 |
| | EphA2#8 | 224,373 | 11,756 | | | | |
| | HEK293- | 2,864,233 ± | 295,696 ± | 9.69 | −7.8 | 89.1 | 14.6 |
| | EphA2#2 | 409,871 | 9,053 | | | | |
| | HEK293- | 4,608,433 ± | 278,564 ± | 16.54 | −11.7 | 87.6 | 9.2 |
| | EphA2#31 | 50,555 | 2,880 | | | | |
| Transiently | HEK293- | 466,203 ± | 296,677 ± | 1.57 | −7.5 | 70.2 | −3.4 |
| ALCAM- | siRNA1 | 82,224 | 63,749 | | | | |
| siRNA/ | HEK293- | 377,816 ± | 208,304 ± | 1.81 | −14.0 | 69.7 | −6.0 |
| EphA2- | SiRNA2 | 95,592 | 23,759 | | | | |
| transfected | HEK293- | 507,760 ± | 60,512 ± | 8.39 | −6.6 | 87.5 | −4.4 |
| HEK293 | siRNA3 | 17,793 | 5,047 | | | | |

### Example 4

### Expanding Beyond Model Cell Lines: Regulation of Internalization Dynamics in Tumor Cells by a Guide/Effector-Based Bispecific

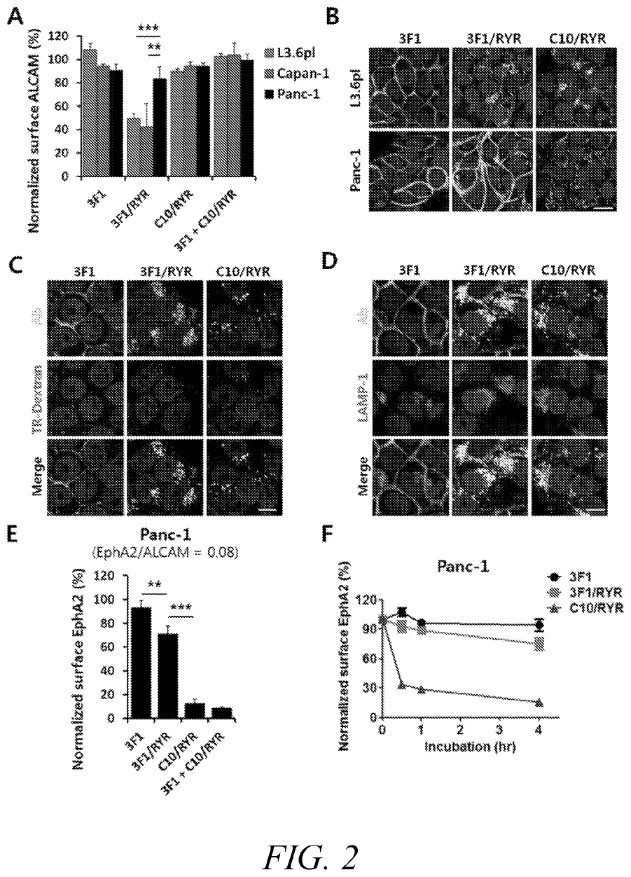

This Examples shows bispecific-induced surface antigen dynamics in a panel of pancreatic cancer cell lines with varying guide to effector ratios. Cell surface antigen density of ALCAM and EphA2 by quantitative FACS was first determined (Table 2). ALCAM was found highly expressed by those cells, and the guide to effector (EphA2 to ALCAM) ratio for L3.6pl, Capan-1, and Panc-1 was estimated to be 0.31, 0.23, and 0.08, respectively. Two sets of experiments were next performed to determine (1) how the non-internalizing ALCAM is converted into an internalizing antigen by the bispecific with an EphA2 to ALCAM ratio above the threshold (>0.2); and (2) how the rapidly internalizing EphA2 is rendered slowly internalizing by the bispecific with an EphA2 to ALCAM ratio below the threshold (<0.2). The internalization dynamics of EphA2 and ALCAM were studied by measuring surface antigen level by FACS following antibody treatment. With regard to ALCAM, the bispecific 3F1/RYR was effective in removing about 60% cell-surface ALCAM in both L3.6pl and Capan-1 cells where the guide to effector ratios are >0.2, but ineffective in Panc-1 cells where the ratio is 0.08, suggesting a cell type selectivity based on the guide to effector ratio (FIG. 2A).

The non-internalizing monoclonal anti-ALCAM antibody 3F1 did not remove any ALCAM antigen from the cell surface. The control C10/RYR or an antibody mixture of 3F1 and C10/RYR removed about 85% of surface EphA2 (FIG. 8C) but failed to remove ALCAM (FIG. 2A), suggesting that ALCAM removal is a bispecific-dependent phenomenon not achievable by oligoclonal antibody mix. The above bispecific effect on antigen internalization was also studied by confocal microscopy. As shown in FIG. 2B, in L3.6pl cells where the EphA2/ALCAM ratio was >0.2 (˜0.31), the anti-ALCAM 3F1 was mostly detected on the cell surface, while the bispecific 3F1/RYR was detected mainly in the cytoplasm with some staining of the cell membrane. The control C10/RYR that binds to EphA2 was detected mainly in the cytoplasm, consistent with its ability to induce rapid EphA2 internalization. In contrast, for the Panc-1 cell line that has an EphA2/ALCAM ratio <0.2 (˜0.08), the bispecific 3F1/RYR was detected mainly on the cell surface (FIG. 2B), again suggesting that internalization of the bispecific depends on the EphA2/ALCAM ratio. Those data confirm that in the guide-effector bispecific design described herein, the ability of the bispecific to convert a non-internalizing to internalizing effector antigen depends on the guide to effector ratio.

TABLE 2
Summary of antigen density and EphA2/ALCAM
ratio in various cell lines studied.

| | Antigen copy number | | |
|---|---|---|---|
| | (mean ± SD, n ≥ 2) | | EphA2/ALCAM |
| Cell lines | EphA2 | ALCAM | ratio |
| L3.6pl | 307,613.74 ± | 1,007,958.84 ± | 0.30518 |
| | 51,947.9 | 374,560.7 | |
| Capan-1 | 148,779.02 ± | 658,071.60 ± | 0.22608 |
| | 36,170.7 | 49,707.2 | |
| Panc-1 | 143,158.43 ± | 1,864,993.96 ± | 0.07676 |
| | 43,021.7 | 107,327.2 | |
| MIA PaCa2* | 44,555,09 ± | ND | ND |
| | 18,731.0 | | |
| C4-2B | 9,690.80 ± | 562,316.24 ± | 0.01724 |
| | 1,022.8 | 53,897.5 | |

*ALCAM copy number is detected as low as that of a control IgG.
ND: not determined (due to low/non ALCAM expression on MIA PaCa2).

To assess pathway of internalization and trafficking to the lysosome, confocal microscopy studies was performed using L3.6pl cells. As shown in FIG. 2C, the bispecific 3F1/RYR was colocalized with the macropinocytosis marker, 70 kDa Neutral Dextran (ND70), suggesting the mode of internalization is macropinocytosis. Post internalization, 3F1/RYR and the control C10/RYR were colocalized with the lysosomal marker lysosomal-associated membrane protein 1 (LAMP1) (FIG. 2D), suggesting that the anti-EphA2 antibody-guided bispecific traffics to the lysosome.

To investigate the other direction of the interconversion between internalization and non-internalization, i.e., conversion of a rapidly internalizing antibody into a slowly or non-internalizing antibody, surface removal of EphA2 by the bispecific in the presence of the neighboring non-internalizing antigen ALCAM was studied. As shown in FIG. 2E, on Panc-1 cell where the EphA2 to ALCAM ratio is <0.2 (˜ 0.08), EphA2 remains mainly on the cell surface when targeted by the bispecific 3F1/RYR. The control C10/RYR removes EphA2 from the cell surface. The C10/RYR plus 3F1 mixture does not retard EphA2 internalization, suggesting that the phenomenon is dependent on the bispecific antibody. A time course internalization study shows that the 3F1/RYR greatly retarded EphA2 internalization kinetics, whereas the control C10/RYR induced rapid EphA2 internalization (FIG. 2F). These studies demonstrate that EphA2 internalization can be significantly retarded by the bispecific when ALCAM is present on the same cell with amounts exceeding the threshold value of EphA2/ALCAM ratio.

### Example 5

### The Bispecific 3F1/RYR Inhibits Pancreatic Tumor-Sphere Formation

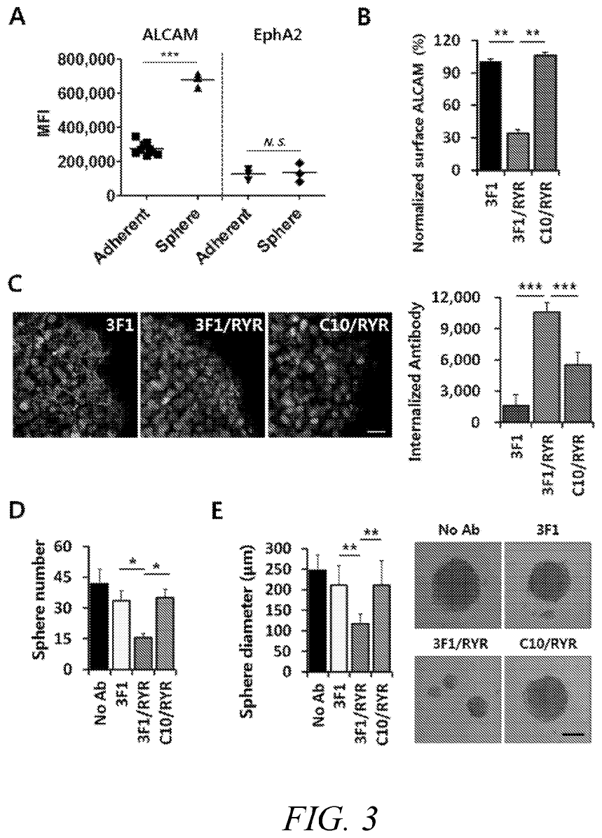

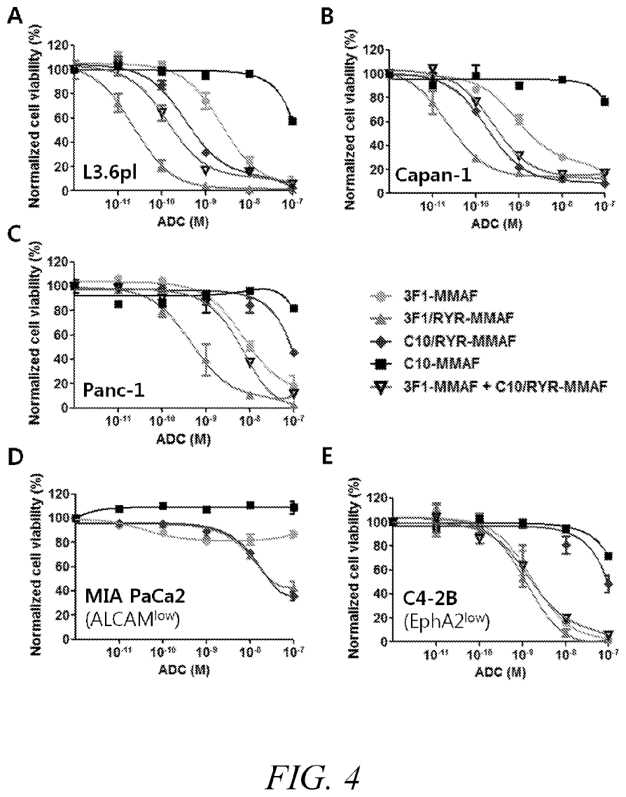

Effects of the anti-EphA2-guided bsIgG (3F1/RYR) on survival and expansion of pancreatic tumor spheres were studied to assess functional consequences of surface antigen removal. Previous studies have shown that cancer cells overexpressing ALCAM aggressively form tumor-spheres, suggesting that ALCAM plays a role in tumor clonogenicity. In this Example, experiments were therefore performed to study whether ALCAM removal by the tetravalent ALCAMxEphA2 bsIgG can inhibit pancreatic tumor sphere formation. First, antigen expression was assessed in L3.6pl tumor spheres and it was found that ALCAM was significantly upregulated on the surface of sphere-forming tumor cells (FIG. 3A). There was no difference of EphA2 surface level between L3.6pl cells in monolayer vs. sphere states (FIG. 3A). Following incubation of L3.6pl tumor spheres with antibodies for 2 weeks, the bispecific 3F1/RYR reduced ALCAM surface density by 70%, whereas the anti-ALCAM mAb 3F1 or the control C10/RYR that binds to EphA2 only showed no effect on ALCAM surface level (FIG. 3B). Confocal microscopy study was next performed to confirm antibody internalization. As shown in FIG. 3C, 3F1/RYR was effectively internalized in L3.6pl sphere forming cells. In contrast, the monoclonal anti-ALCAM antibody 3F1 showed mainly surface staining (FIG. 3C). The control bispecific C10/RYR was internalized, consistent with its monospecific binding to EphA2 (FIG. 3C). Remarkably, based on the fluorescence signal intensity per cell, a greater amount of the 3F1/RYR was taken up by tumor sphere-forming cells compared with the control bispecific C10/RYR (FIG. 3C, right panel), suggesting an amplification effect unique to the bispecific antibody. With regard to functional effect on tumor clonogenic activity, it was found that L3.6pl sphere number (FIG. 3D) and size (FIG. 3E) was significantly decreased by treatment with 3F1/RYR but not 3F1 or C10/RYR, consistent with previous studies of the role of ALCAM in tumor sphere formation and growth. Thus the bispecific antibody with one arm binding to the internalizing antigen EphA2 can effectively remove the non-internalizing antigen ALCAM from tumor cell surface, resulting in inhibition of pancreatic tumor sphere growth.

### Example 6

### Potent and Cell-Type Selective In Vitro Tumor Cell Killing by Bispecific ADC

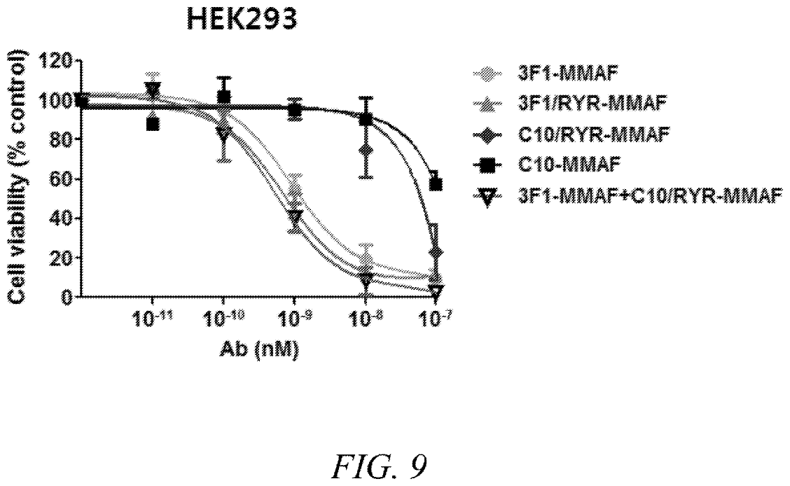

To explore therapeutic potential of the bispecific-induced amplification of intracellular uptake, a number of monospecific and bispecific ADCs were generated by site-specific conjugation of MC-VC-pab-MMAF, analyzed conjugation products by HIC-HPLC, and determined the drug-to-antibody ratio (˜1.9). In these experiments, ADCs were tested on a panel of cancer cell lines that display different levels of cell-surface EphA2 and ALCAM, and EphA2 to ALCAM ratios (see, e.g., Table 2). The 3F1/RYR ADC showed potent cytotoxicity with EC50 of 23 pM on L3.6pl and 22 pM on Capan-1 cells (FIGS. 4A and 4B, and Table 3). These two cell lines have EphA2 to ALCAM ratios above the threshold (0.2, Table 2), resulting in more efficient internalization. In contrast, both 3F1 and C10/RYR ADCs showed lower potency. EC50 values of 3F1 and C10/RYR ADCs on L3.6pl are 2.37 nM and 0.35 nM respectively, and on Capan-1 are 0.87 nM and 0.18 nM, respectively (FIGS. 4A and 4B). Most remarkably, the bispecific ADC is more potent than the mix of monoclonal ADCs (C10/RYR ADC+3F1 ADC, FIGS. 4A and 4B), suggesting again that this enhanced potency is unique to the bispecific antibody. Cytotoxicity of ADCs on Panc-1 cells with a low guide to effector (EphA2 to ALCAM) ratio and MIA PaCa2 cells without the effector antigen (ALCAM) expression was also studied. On Panc-1 cells with a low EphA2/ALCAM ratio (0.08), the bispecific 3F1/RYR ADC showed reduced potency (EC50=0.46 nM) but is still more potent than 3F1 (EC50=9.3 nM) and C10/RYR ADCs (EC50>100 nM) (FIG. 4C and Table 3). Again, the cytotoxic potency of 3F1/RYR ADC was higher than a mixture of the 3F1 and C10/RYR ADCs (EC50=0.46 nM vs. 7.14 nM for the mix) (Table 3). On the ALCAM-negative MIA PaCa2 cell line, the 3F1 ADC showed little cytotoxicity as expected (FIG. 4D). 3F1/RYR and C10/RYR ADCs showed similarly low cytotoxicity due to the lack of expression of ALCAM and low expression level of EphA2 (FIG. 4D). To further evaluate cell type selectivity, LNCaP-C4-2B and HEK293 cell lines, which express very low levels of EphA2, was studied. On LNCaP-C4-2B, as shown in FIG. 4E, the bispecific 3F1/RYR ADC did not show enhanced cytotoxicity vs. the monoclonal 3F1 ADC (EC50=1.25 nM vs. 1.64 nM, Table 3), due to the lack of the guide antigen EphA2 expression, showing a guide antigen-dependent cell type selectivity. Similar results were obtained from studies using HEK293 cells that lack the guide antigen expression (FIG. 9). Taken together, those data show that the bispecific ADC is more potent than mono-specific ADCs or their mixture, and exhibits a cell-type selective potency enhancement depending on the guide to effector antigen ratio.

TABLE 3
In vitro potency of ADCs or mix of ADCs on tumor
cell lines with varying EphA2/ALCAM ratios.

| | | | IC50 (nM) | | | |
|---|---|---|---|---|---|---|
| | | | | | 3F1-MMAF + | |
| | | E/A | | C10/RYR- | C10/RYR- | 3F1/RYR- |
| Cell lines | | ratio | 3F1-MMAF | MMAF | MMAF | MMAF |
| Pancreatic | L3.6pl | 0.31 | 2.372 | 0.345 | 0.135 | 0.023 |
| cancer cell lines | Capan-1 | 0.23 | 0.866 | 0.178 | 0.248 | 0.022 |
| | Panc-1 | 0.08 | ~10 | ~100 | 2.539 | 0.261 |
| Prostate cancer | C4-2B | 0.017 | 1.642 | ~100 | 1.371 | 1.247 |
| cell lines | | | | | | |

### Example 7

### In Vivo Anti-Tumor Efficacy of the ALCAMxEphA2 Bispecific ADC

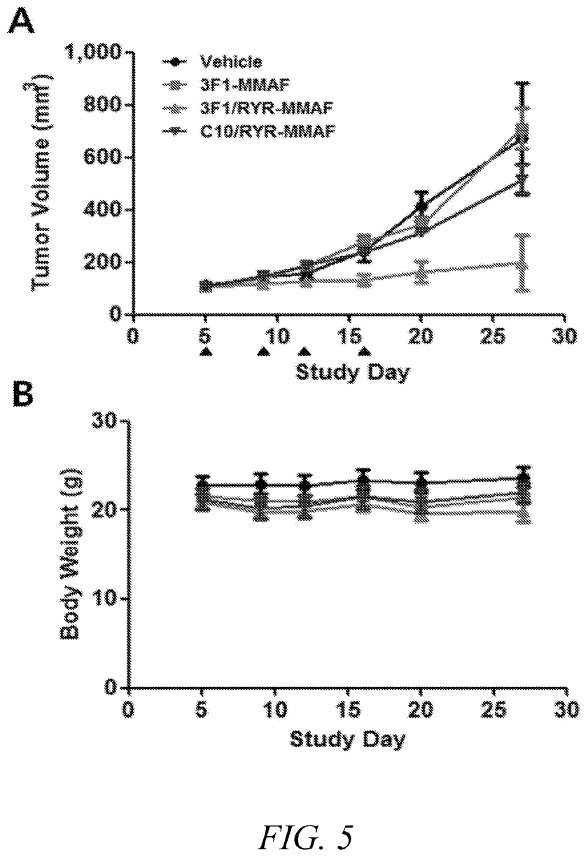

This Example summarizes experiments performed to study in vivo efficacy of the bispecific 3F1/RYR ADC along with control ADCs on pancreatic cancer xenografts. Capan-1 cells were implanted subcutaneously into NSG mice. When the tumor reached an average volume of 110 mm3, 3F1/RYR, 3F1, or C10/RYR ADCs were injected at 3 mg/kg every four days for a total of four times. Tumor status was monitored by caliper measurement. Overt toxicity was monitored by body weight loss. As shown in FIG. 5A, the bispecific 3F1/RYR ADC significantly inhibited tumor growth, while the monoclonal 3F1 ADC or the control bispecific C10/RYR ADC had only a moderate effect on tumor size reduction. There was no significant change of body weights during the course of the study for any of the ADCs studied (FIG. 5B). These data demonstrate that in the guide-effector bispecific design described herein, the rapidly internalizing anti-guide (EphA2) scFv can induce internalization of the otherwise non-internalizing effector antigen (ALCAM), resulting in greater amount of bispecific ADCs taken into the tumor cells compared to monospecific ADCs, thus showing enhanced anti-tumor efficacy in vivo.

### Example 8

### Cell Lines and Plasmids

Human embryonic kidney (HEK) lines HEK293 and HEK293A; prostate cancer cell line DU145 and PC3; and pancreatic cancer cell lines Capan-1, Panc-1 and MIA PaCa2 were obtained from American Type Culture Collection (ATCC). The L3.6pl line was obtained from Dr. Isaiah Fidler (MD Anderson Cancer Center, Houston, Tex.). The LNCap-C4-2B was originally obtained from UroCor Inc. and maintained in the laboratory. Cells were maintained in DMEM or RPMI1640 supplemented with 10% FBS (Fisher Scientific), 100 μg/ml penicillin/streptomycin (Axenia BioLogix) at 37° C., 5% CO₂. Full-length human EphA2 cDNA cloned into pCMV-Entry (Origene) or pLV202 (Origene) was used for transient or stable expression of EphA2, respectively.

### Example 9

### Generation of Anti-ALCAM scFv Antibodies

A naïve scFv-phagemid display library was used for antibody selection. A recombinant human IgG-like V1-V2 domain of ALCAM fused with human IgG2 Fc (A-V-Fc) was produced from HEK293A cells and utilized as an antigen. A-V-Fc was coated on SPHERO™ polystyrene magnetic particles (Spherotech) at 4° C. for overnight. Phage library was depleted with uncoated beads in PBS/2% milk, and unbound phages allowed bind to the A-V-Fc-coated beads. The beads were then washed, eluted, and propagated as described previously. Individual phage binders were screened by FACS using the ALCAM-expressing DU145 cell line, and DNA sequence of scFvs was analyzed by IgAT tool.

### Example 10

### Generation of Anti-EphA2 scFv Antibodies

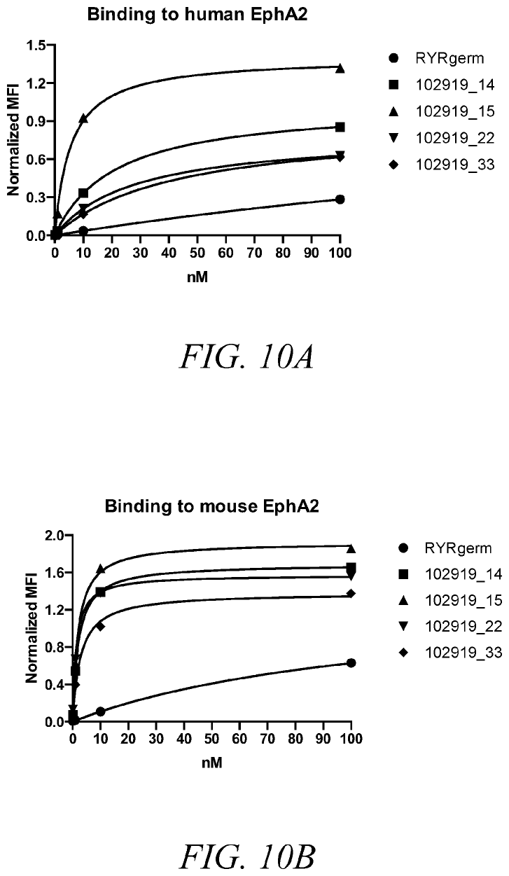

This Example describes experiments performed to identify new versions of EphA2 binding scFv antibodies with improved binding affinity. The original EphA2 binding scFv RYR has been described previously in PCT/US2015/039741, in which EphA2 binding scFv RYR was named HCA-F1 and a germline version of RYR was named RYRgerm. To identify new versions of EphA2 binding scFv antibodies improved binding affinity for EphA2, yeast display mutagenesis libraries based on RYRgerm were generated and selected by FACS for higher affinity binders. Four new EphA2 scFvs with high binding affinity for EphA2 were identified and named RYRgerm_102019_14, RYRgerm_102919_15, RYRgerm_102919_22, and RYRgerm_102919_33, respectively. The amino acid sequences of VH and VL regions as well as the CDRs for these newly identified EphA2 scFvs are presented in Tables 4-5 and the Sequence Listing. In these eperiments, both human and mouse recombinant EphA2 proteins were used in selection to maintain cross-species binding. Apparent affinity was measured by flow cytometry for binding to both human and mouse EphA2. As shown in FIG. 10A, the new versions of EphA2 binding scFv antibodies identified in yeast display mutagenesis libraries demonstrated an enhanced binding affinity for human EphA2 when compared to the original EphA2-binding scFv RYR, with about 8-fold to about 70-fold increase in binding affinity. The apparent K_{D} values for human EphA2 binding affinity were: RYRgerm: 354.9 nM (original EphA2 scFv); RYRgerm_102019_14: 21.27 nM; RYRgerm_102919_15: 5.58 nM; RYRgerm_102919_22: 28.13 nM; RYRgerm_102919_33: 42.49 nM. Similarly, as shown in FIG. 10B, the new versions of EphA2 binding scFv antibodies identified in yeast display mutagenesis libraries demonstrated an improved binding affinity for mouse recombinant EphA2-Fc when compared to the original EphA2 binding scFv RYR, with about 40-fold to about 80-fold increase in binding affinity. The apparent K_{D} values for mouse recombinant EphA2-Fc fusion binding affinity were: RYRgerm (original EphA2 scFv): 114.7 nM; RYRgerm_102019_14: 2.12 nM; RYRgerm_102919_15: 2.01 nM; RYRgerm_102919_22: 1.34 nM; RYRgerm_102919_33: 2.87 nM.

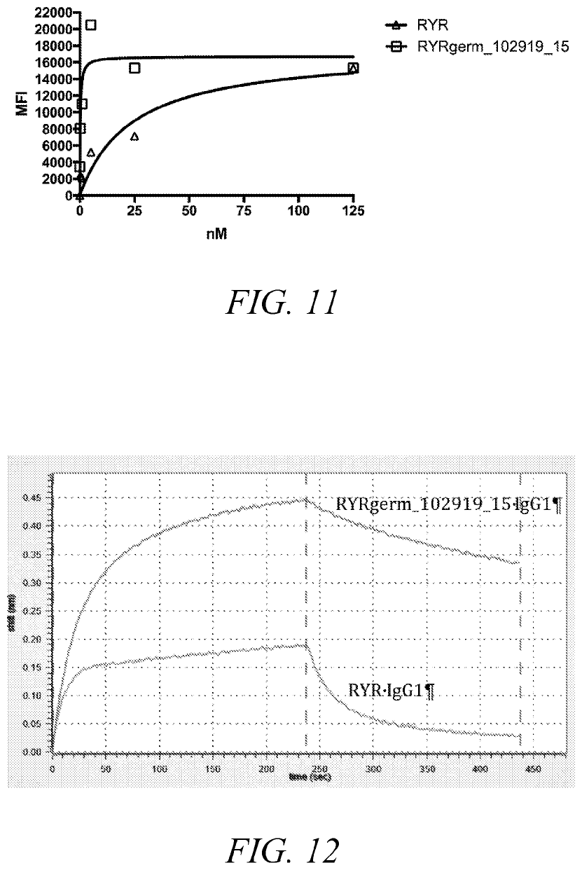

Subsequently, in order to go beyond the assessment of scFv binding activity in yeast cells, additional recombinant human IgG1s were designed and constructed with the original EphA2 scFv (RYR) and the newly improved RYRgerm_102919_15, and studied their binding affinity on living cells and recombinant antigens. FIG. 11 summarizes the results of experiments performed in human prostate cancer cell lines DU145 to compare the affinities of recombinant IgG1s between the original RYR and the newly improved RYR-binding scFv RYRgerm_102919_15 described in FIGS. 10A-10B. In these experiments, apparent binding affinity of RYR IgG1 versus RYRgerm_102919_15 IgG1 on DU145 cells was evaluated. In these experiments, DU145 cells were incubated for 1 h at 25° C. with RYR or RYRgerm_102919_15 at a range of concentrations between 40 pM to 125 nM, washed, and binding detected with anti-human Alexa Fluor 647. MFI values were curve-fit to generate the apparent K_{D} values. The K_{D} values for RYR IgG1 and RYRgerm_102919_15 were 23.7 nM and 0.23 nM, respectively, which indicates an approximately 100-fold increase in binding affinity.

Additional experiments were also performed to evaluate the binding affinity of the new EphA2 scFv RYRgerm_102919_15 on recombinant human EphA2 (see, e.g., FIG. 12. In these experiments, label-free Biolayer interferometry (BLI) analysis was performed using a Probe Life Gator instrument. Anti-human Fc probes were loaded with RYR or RYRgerm_102919_15 IgG1 and 100 nM recombinant human EphA2 (R&D System) was used for the binding assay at 25° C. The apparent affinity K_{D} for the RYR IgG1 was ˜ 28 nM (K_{off}/Kₒₙ=1.45E−02/5.18E+05), whereas the apparent affinity K_{D} for the improved RYRgerm_102919_15 IgG1 was ˜5.0 nM (K_{off}/Kₒₙ=1.32E−03/2.62E+05), indicating an approximately 5-fold increase in binding affinity.

### Example 11

### Recombinant Antibody Production

VH and VL antibody genes were amplified from candidate scFv-phagemids by PCR and sub-cloned into Abvec Ig-γ and -λ expression vectors, respectively. To produce bispecific IgG-scFvs, the anti-ALCAM 3F1 or a non-binding control C10 was utilized as the IgG backbone, and the internalizing scFv was introduced at C-terminus of the λ light chain constant region (CL) by fusion with a (Gly4Ser)₃ linker. HEK293A cells were transfected with antibody expression plasmids mixed with polyethylenimine (Sigma Aldrich) in Opti-MEM (Life Technologies) for 24 hours. Transfection medium was changed to Freestyle® 293 (Gibco) and the cells were further cultured up to 8 days. Secreted antibodies were purified from culture supernatants on protein A agarose (Thermo Scientific) and analyzed on SDS-PAGE gradient gels (4-20%).

### Example 12

### Generation of Stable HEK293-EphA2 Cell Line

HEK293 cells were transduced with EpAh2-expressing lentiviral vector and maintained in regular growth medium containing G418 (Sigma). Stable EphA2-expressing clones were identified by FACS using human anti-EphA2 antibody followed by Alexa Fluor® 647-labeled goat anti-human IgG (Jackson ImmunoResearch). Stable clones were further screened by FACS to obtain those that express varying levels of EphA2.

### Example 13

### Cell Surface Antigen Copy Number Measurement

Cell surface antigen copy number (or antigen density) was measured as described previously. Briefly, cells were dissociated by 0.25% trypsin digestion, washed and resuspended in FACS assay buffer (PBS, 1% FBS, pH 7.4), incubated with anti-EphA2 or ALCAM antibodies that were conjugated with Alexa Fluor® 647 by Monoclonal Antibody Labeling Kit (Molecular Probes) to detect EphA2 or ALCAM, respectively, and analyzed by BD Accuri C6 (BD Biosciences). Median Fluorescence Intensity (MFI) was converted into Antibody Binding Capacity (ABC) using Quantum™ Alexa Fluor® 647 MESF and Quantum™ Simple Cellular® anti-human IgG (Bang's Laboratory) according to manufacturer's recommendations. E/A (EphA2/ALCAM) ratios were calculated by dividing the copy number of EphA2 by that of ALCAM for each cell model studied.

### Example 14

### Apparent K_{D} Determination

Dissociated cells (˜2×10⁵) were incubated with varying concentrations of human IgGs for 16 hours at 4° C. Following three washes with ice-cold PBS, cell-bound IgG was detected by Alexa Fluor® 647-labeled goat anti-human IgG (Jackson ImmunoResearch) and analyzed by FACS. Apparent K_{D} value was calculated by a curve-fitting method using GraphPad Prism software.

### Example 15

### Cell Surface Antigen Depletion

Mono- or bi-specific antibodies (100 nM) were incubated with cells cultured in 24-well plates (˜80% confluence) for 24 hours, and EphA2 or ALCAM remaining on cell surface was determined using Alexa Fluor® 647-labeled L1A1 anti-EphA2 human IgG or L50 anti-ALCAM mouse IgG (Fisher Scientific), respectively. Cell-surface copy number was calculated using methods described above and normalized against a control group without antibody treatment.

### Example 16

### Immunofluorescence Confocal Microscopy

Antibodies were incubated with cells seeded in 8-well culture chamber slides (Fisher Scientific) for the indicated amount of time. To assess pathway of internalization (macropinocytosis), cells were co-incubated with TexasRed-conjugated 70-kDa neutral dextran (ND70-TR, Life Technologies), a marker for macropinocytosis. Post incubation cells were fixed with 4% paraformaldehyde (PFA) and permeabilized with PBS/1% FBS/0.2% Triton-X100. Cell-associated antibodies were stained with Alexa Fluor® 488- or 647-labeled goat anti-human IgG (Jackson ImmunoResearch) for 1 hour at room temperature. Lysosomes were detected by rabbit anti-lysosomal-associated membrane protein 1 (LAMP1) antibody (Cell Signaling) followed by incubation with Alexa Fluor® 647-labeled goat anti-rabbit IgG (Jackson ImmunoResearch). For analysis of antibody localization in tumor spheres, spheres were collected by centrifugation at 500×g for 5 min, washed, fixed, permeabilized, and immunolabeled using antibodies described above. CyGEL® (Abcam) was used to immobilize spheres in 8-well chamber slide for microscope analysis. For imaging, cells or spheres were counterstained using Hoechst 33342 (Thermo Scientific) and imaged by FluoView® FV10i laser confocal microscope (Olympus) with an Olympus 60X phase contrast water-immersion objective.

### Example 17

### Tumor Sphere Formation

Tumor spheres were generated by culturing suspended tumor cells from monolayer culture in serum free medium (SFM) containing DMEM/F12 (Gibco), 20 ng/ml EGF, 10 ng/ml bFGF, 10 ng/ml IGF and 2% B27 supplement (Gibco) in ultra-low attachment 24-well plate (Corning) at 37° C./5% CO2. For sphere propagate assay, the first generation spheres were trypsinized and sieved through a 40-μm nylon mesh cell strainer (Fisher Scientific) to obtain a single cell population. 200 cells per well were resuspended in 500 μl SFM, seeded in ultra-low attachment 24-well plate (Corning) at 37° C./5% C02 for 24 hours, and treated with indicated antibodies for 2 weeks. Cells were fed with 100 μl SFM every 3-4 days. Each well was sectionally scanned using the BIOREVO digital microscope (BZ-9000; Keyence) and merged to display the whole well image. Spheres >100 μm in diameter were counted.

### Example 18

### Site-Specific ADC Generation

A cysteine residue was introduced to heavy chain position 116 (T116C) of IgG or bsIgG, and site-specific ADCs were generated as described previously with modifications. Briefly, antibodies in PBS were reduced by incubating with 10-fold molar excess of Tris(2-carboxyethyl)phosphine hydrochloride (TCEP) (Thermo Scientific) at 37° C. for 2 hours, purified by Zeba spin desalting column (Thermo Scientific) and buffer-exchanged in PBS/5 mM EDTA. To re-oxidize inter-chain disulfide bonds, reduced antibodies were incubated with 20-fold molar excess of dehydroascorbic acid (dhAA) (Sigma) at 25° C. for 3 hours. After buffer-exchange with PBS/5 mM EDTA, the antibody was incubated at 25° C. for 1 hour with 3-fold molar excess of maleimidocaproyl-valine-citrulline-p-aminobenzoyloxycarbonyl monomethyl auristatin F (MC-vc-PAB-MMAF) that was synthesized as previously described. The final conjugation product was purified by running twice through the Zeba™ spin desalting column (Fisher Scientific) and analyzed by hydrophobic interaction chromatography (HIC)-HPLC using the infinity 1220 LC System (Agilent). Drug to antibody ratio (DAR) is estimated from area integration using the OpenLab CDS software (Agilent).

### Example 19

### ADC Cytotoxicity

Cells were seeded at 2×10³/well in 96-well cell culture plates overnight, and incubated with varying concentrations of ADCs for 96 hours. Cell viability was determined using a Calcein-AM cell viability assay kit (Biotium Inc.).

### Example 20

### In Vivo Xenograft Study

All animal studies were approved by the UCSF Animal Care and Use Committee (AN092211) and conducted in adherence to the NIH Guide for the Care and Use of Laboratory Animals. NOD/SCID/IL-2Rγ^{-/-} (NSG) female mice were engrafted with 1×10⁶ Capan-1 cells, randomized into 4 groups at day 5 (n=6 for each group). Mice were treated intravenously with the vehicle PBS or mono- or bi-specific ADCs at 3 mg/kg every 4 days for a total of 4 injections. Tumor size was measured by a caliper, and tumor volume was calculated using the formula V=(Width²×Length)/2. Body weight was monitored during the course of the study.

TABLE 4
Amino acid sequences of exemplary engineered antibodies in accordance to
some non-limiting embodiments of the disclosure. The amino acid sequences of VH and
VL are shown. Regions corresponding to CDR1, CDR2 and CDR3, respectively, are indicated
in the Sequence Listing.

| Antibody | VH | VL | Note |
|---|---|---|---|
| Original 1-13 | QVQLVESGGGLVQPGGSLRLSCAASG | NFMLTQDPAVSVALGQTVRITCQG | |
| (aka | FTFSSYAMSWVRQAPGKGLEWVSAIS | DSLRSYYASWYQQKPGQAPLLVIY | |
| 585II41.1) | GSGGSTYYADSVKGRFTISRDNSKDT | GKNNRPSGIPDRFSGSSSGNTASL | |
| | LYLQMNSLRAEDTAVYYCASRSLLDY | TITGAQAEDEADYYCNSRDSSGNP | |
| | WGQGTLVTVSS | VFGGGTKVTVL | |
| | (SEQ ID NO: 1) | (SEQ ID NO: 2) | |
| H3germ | EVQLVESGGGLVQPGGSLRLSCAASG | SSELTQDPAVSVALGQTVRITCQG | Similar to H3 |
| | FTFSSYAMSWVRQAPGKGLEWVSAIS | DSLRSYYASWYQQKPGQAPVLVIY | with |
| | GSGGSTYYADSVKGRFTISRDNSKNT | GKNNRPSGIPDRFSGSSSGNTASL | differences in |
| | LYLQMNSLRAEDTAVYYCASRSLLDY | TITGAQAEDEADYYCNSRDSSGNP | VH and VL |
| | WGQGTLVTVSS | VFGGGTKVTVL | underlined |
| | (SEQ ID NO: 3) | (SEQ ID NO: 4) | |
| H3like | EVQLVESGGGLVQPGGSQRLSCAASG | QSALTQDPAVSVALGQTVRITCQG | Similar to H3 |
| 01D_ALCNK2 | FTFSSYAMSWVRQAPGKGLEWVSAIS | DSLRSYYASWYQQKPGQAPLLVIY | with |
| | GSGGSTYYADSVKGRFTISRDNSKDT | GKNNRPSGIPDRFSGSSSGNTASL | differences |
| | LYLQMNSLRAEDTAVYYCASRSLLDY | TITGAQAEDEADYYCNSRDSSGNP | in VH and VL |
| | WGQGTLVTVSS | VFGGGTKLTVL | underlined |
| | (SEQ ID NO: 5) | (SEQ ID NO: 6) | |
| H3like | EVQLVESGGGLVQPGGSLRLSCAASG | QSVLTQDPAVSVALGQTVRITCQG | Similar to H3 |
| 03E_ALCNK2 | FTFSSYAMSWVRQAPGKGLEWVSAIS | DSLRSYYASWYQQKPGQAPVLVIY | with |
| | GSGGSTYYADSVKGRFTISRDNSKDT | GKNNRPSGIPDRFSGSSSGNTASL | differences |
| | LYLQMNSLRAEDTAVYYCASRSLLDY | TITGAQAEDEADYYCNSRDSSGNH | in VH and VL |
| | WGQGTLVTVSS | VFGGGTQLTVL | underlined |
| | (SEQ ID NO: 7) | (SEQ ID NO: 8) | |
| H3like | QVQLVESGGGLVQPGGSLRLSCAASG | NFMLTQDPAVSVALGQTVRITCQG | Same VH as of |
| 04E_ALCNK2 | FTFSSYAMSWVRQAPGKGLEWVSAIS | DSLRSYYASWYQQKPGQAPVLVIY | H3; |
| | GSGGSTYYADSVKGRFTISRDNSKDT | GKNNRPSGIPDRFSGSSSGNTASL | difference in |
| | LYLQMNSLRAEDTAVYYCASRSLLDY | TITGAQAEDEADYYCNSRDSSGNH | VL underlined |
| | WGQGTLVTVSS | LFGGGTKLTVL | |
| | (SEQ ID NO: 9) | (SEQ ID NO: 10) | |
| H3like | QVQLVESGGGLVQPGGSLRLSCAASG | NFMLTQDPAVSVALGQTVRITCQG | Same VH as of |
| 02F_ALCNK2 | FTFSSYAMSWVRQAPGKGLEWVSAIS | ESLRRYYGSWYHQRPGQAPLLVFY | H3; |
| | GSGGSTYYADSVKGRFTISRDNSKDT | GKNRRPSGIPDRFSGSSSGDTATL | difference in |
| | LYLQMNSLRAEDTAVYYCASRSLLDY | TITGAQAEDEGAFYCNSRDGSANH | VL underlined |
| | WGQGTLVTVSS | FFGGGTQLTVL | |
| | (SEQ ID NO: 11) | (SEQ ID NO: 12) | |
| 08D_ALCNK2 | QVQLVESGGGLVQPGGSLRLSCAASG | QSALTQPASVSGSPGQSITISCTG | Same VH as H3 |
| | FTFSSYAMSWVRQAPGKGLEWVSAIS | TSTDIGYYNYVSWYQQHPGKAPKL | but a |
| | GSGGSTYYADSVKGRFTISRDNSKDT | MIYDVSKRPSGVSNRFSGSKSGNT | different VL |
| | LYLQMNSLRAEDTAVYYCASRSLLDY | ASLTISGLQAEDEADYYCSSFTSS | |
| | WGQGTLVTVSS | TTYVFGTGTQLTVL | |
| | (SEQ ID NO: 13) | (SEQ ID NO: 14) | |
| 08D_ALCNK2_ | EVQLVESGGGLVQPGGSLRLSCAASG | QSALTQPASVSGSPGQSITISCTG | VH Germline |
| germ | FTFSSYAMSWVRQAPGKGLEWVSAIS | TSTDIGYYNYVSWYQQHPGKAPKL | variant of |
| | GSGGSTYYADSVKGRFTISRDNSKNT | MIYDVSKRPSGVSNRFSGSKSGNT | 08D_ALCNK2 |
| | LYLQMNSLRAEDTAVYYCASRSLLDY | ASLTISGLQAEDEADYYCSSFTSS | |
| | WGQGTLVTVSS | TTYVFGTGTQLTVL | |
| | (SEQ ID NO: 3) | (SEQ ID NO: 14) | |
| 02B_ALCNK1 | QVQLVESGGGLVQPGGSLRLSCAASG | QSALTQPASMSGSPGQSITMSCTG | Same VH as H3 |
| | FTFSSYAMSWVRQAPGKGLEWVSAIS | TSSDVGGSNYVSWYQQHPGKAPKL | but a |
| | GSGGSTYYADSVKGRFTISRDNSKDT | IIYDVTNRPSGVSNRFSGSKSGNT | different VL |
| | LYLQMNSLRAEDTAVYYCASRSLLDY | ASLTISGLQAEDEADYYCSSFTSS | |
| | WGQGTLVTVSS | TTLVVFGGGTKITVL | |
| | (SEQ ID NO: 15) | (SEQ ID NO: 16) | |
| 02B_ALCNK1_ | EVQLVESGGGLVQPGGSLRLSCAASG | QSALTQPASMSGSPGQSITMSCTG | VH Germline |
| germ | FTFSSYAMSWVRQAPGKGLEWVSAIS | TSSDVGGSNYVSWYQQHPGKAPKL | variant of |
| | GSGGSTYYADSVKGRFTISRDNSKNT | IIYDVTNRPSGVSNRFSGSKSGNT | 02B_ALCNK1 |
| | LYLQMNSLRAEDTAVYYCASRSLLDY | ASLTISGLQAEDEADYYCSSFTSS | |
| | WGQGTLVTVSS | TTLVVFGGGTKITVL | |
| | (SEQ ID NO: 3) | (SEQ ID NO: 16) | |
| 03F_ALCNK2 | QVQLVESGGGLVQPGGSLRLSCAASG | QSALTQPASVSGSPGQSITISCTG | Same VH as H3 |
| | FTFSSYAMSWVRQAPGKGLEWVSAIS | TSSDVGGYNYVSWYQQHPGKAPKL | but a |
| | GSGGSTYYADSVKGRFTISRDNSKDT | MIYDVSNRPSGVSNRFSGSKSGNT | different VL |
| | LYLQMNSLRAEDTAVYYCASRSLLDY | ASLTISGLQAEDEADYYCSSFTSS | |
| | WGQGTLVTVSS | STLYVFGTGTQLTVL | |
| | (SEQ ID NO: 17) | (SEQ ID NO: 18) | |
| 03F_ALCNK2_ | EVQLVESGGGLVQPGGSLRLSCAASG | QSALTQPASVSGSPGQSITISCTG | VH Germline |
| germ | FTFSSYAMSWVRQAPGKGLEWVSAIS | TSSDVGGYNYVSWYQQHPGKAPKL | variant of |
| | GSGGSTYYADSVKGRFTISRDNSKNT | MIYDVSNRPSGVSNRFSGSKSGNT | 03F_ALCNK2 |
| | LYLQMNSLRAEDTAVYYCASRSLLDY | ASLTISGLQAEDEADYYCSSFTSS | |
| | WGQGTLVTVSS | STLYVFGTGTQLTVL | |
| | (SEQ ID NO: 19) | (SEQ ID NO: 20) | |
| 10D_ALCNK1 | QVQLVESGGGLVQPGGSLRLSCAASG | QSALTQPASVSGSPGQSITISCTG | Same VH as H3 |
| | FTFSSYAMSWVRQAPGKGLEWVSAIS | TSSDVGGYNYVSWHQQHPGKAPKL | but a |
| | GSGGSTYYADSVKGRFTISRDNSKDT | MIYDVSNRPSGVSNRFSGSKSGNT | different VL |
| | LYLQMNSLRAEDTAVYYCASRSLLDY | ASLTISGLQAEDEADYYCSSYTSS | |
| | WGQGTLVTVSS | SSLYVFGTGTQLTVL | |
| | (SEQ ID NO: 21) | (SEQ ID NO: 22) | |
| 10D_ALCNK1_ | EVQLVESGGGLVQPGGSLRLSCAASG | QSALTQPASVSGSPGQSITISCTG | VH Germline |
| germ | FTFSSYAMSWVRQAPGKGLEWVSAIS | TSSDVGGYNYVSWHQQHPGKAPKL | variant of |
| | GSGGSTYYADSVKGRFTISRDNSKNT | MIYDVSNRPSGVSNRFSGSKSGNT | 10D_ALCNK1 |
| | LYLQMNSLRAEDTAVYYCASRSLLDY | ASLTISGLQAEDEADYYCSSYTSS | |
| | WGQGTLVTVSS | SSLYVFGTGTQLTVL | |
| | (SEQ ID NO: 3) | (SEQ ID NO: 22) | |
| 01F_ALCNK2 | QVQLVESGGGLVQPGGSLRLSCAASG | QSALTQPASVSGSPGQSVTISCTG | Same VH as H3 |
| | FTFSSYAMSWVRQAPGKGLEWVSAIS | TNSDVGAYNYVSWYQQHPGKAPKL | but a |
| | GSGGSTYYADSVKGRFTISRDNSKDT | MIYDVSNRPSGVSNRFSGSKSGNT | different VL |
| | LYLQMNSLRAEDTAVYYCASRSLLDY | ASLTISGLQTEDEADYYCTSYTSS | |
| | WGQGTLVTVSS | NTRVFGGGTQLTVL | |
| | (SEQ ID NO: 23) | (SEQ ID NO: 24) | |
| 01F_ALCNK2_ | EVQLVESGGGLVQPGGSLRLSCAASG | QSALTQPASVSGSPGQSVTISCTG | VH Germline |
| germ | FTFSSYAMSWVRQAPGKGLEWVSAIS | TNSDVGAYNYVSWYQQHPGKAPKL | variant of |
| | GSGGSTYYADSVKGRFTISRDNSKNT | MIYDVSNRPSGVSNRFSGSKSGNT | 01F_ALCNK2 |
| | LYLQMNSLRAEDTAVYYCASRSLLDY | ASLTISGLQTEDEADYYCTSYTSS | |
| | WGQGTLVTVSS | NTRVFGGGTQLTVL | |
| | (SEQ ID NO: 25) | (SEQ ID NO: 26) | |
| 09E_ALCNK2 | QVQLVESGGGLVQPGGSLRLSCAASG | QSALTQPPSASGSPGQSVTISCTG | Same VH as H3 |
| | FTFSSYAMSWVRQAPGKGLEWVSAIS | TSSDVGRYNLVSWYQRHPGKVPKL | but a |
| | GSGGSTYYADSVKGRFTISRDNSKDT | IIYEVTKRPSGVPDRFSGSKSGNT | different VL |
| | LYLQMNSLRAEDTAVYYCASRSLLDY | ASLTVSGLQAEDEADYYCSSHGSS | |
| | WGQGTLVTVSS | NAFYVFGTGTKLTVL | |
| | (SEQ ID NO: 27) | (SEQ ID NO: 28) | |
| 09E_ALCNK2_ | EVQLVESGGGLVQPGGSLRLSCAASG | QSALTQPPSASGSPGQSVTISCTG | VH Germline |
| germ | FTFSSYAMSWVRQAPGKGLEWVSAIS | TSSDVGRYNLVSWYQRHPGKVPKL | variant of |
| | GSGGSTYYADSVKGRFTISRDNSKNT | IIYEVTKRPSGVPDRFSGSKSGNT | 09E_ALCNK2 |
| | LYLQMNSLRAEDTAVYYCASRSLLDY | ASLTVSGLQAEDEADYYCSSHGSS | |
| | WGQGTLVTVSS | NAFYVFGTGTKLTVL | |
| | (SEQ ID NO: 3) | (SEQ ID NO: 28) | |
| 11G_ALCNK2 | QVQLVESGGGLVQPGGSLRLSCAASG | QSALTQPASVSGSPGQSITISCTG | Same VH as H3 |
| | FTFSSYAMSWVRQAPGKGLEWVSAIS | TSRDVGGYDYVSWYQQHPGKAPKF | but a |
| | GSGGSTYYADSVKGRFTISRDNSKDT | IIYDVNKRPSGVSNRFSGSKSGNT | different VL |
| | LYLQMNSLRAEDTAVYYCASRSLLDY | ASLTISGLQPEDEADYICSSFTMY | |
| | WGQGTLVTVSS | STPVIFGGGTQLTVL | |
| | (SEQ ID NO: 29) | (SEQ ID NO: 30) | |
| 11G_ALCNK2_ | EVQLVESGGGLVQPGGSLRLSCAASG | QSALTQPASVSGSPGQSITISCTG | VH Germline |
| germ | FTFSSYAMSWVRQAPGKGLEWVSAIS | TSRDVGGYDYVSWYQQHPGKAPKF | variant of |
| | GSGGSTYYADSVKGRFTISRDNSKNT | IIYDVNKRPSGVSNRFSGSKSGNT | 11G_ALCNK2 |
| | LYLQMNSLRAEDTAVYYCASRSLLDY | ASLTISGLQPEDEADYICSSFTMY | |
| | WGQGTLVTVSS | STPVIFGGGTQLTVL | |
| | (SEQ ID NO: 31) | (SEQ ID NO: 32) | |
| 09C_ALCNK1 | QVQLVESGGGLVQPGGSLRLSCAASG | QSALTQPPSASGSPGQSVTISCTG | Same VH as H3 |
| | FTFSSYAMSWVRQAPGKGLEWVSAIS | TSSDVGGYNYVSWYQRHPGKAPKL | but a |
| | GSGGSTYYADSVKGRFTISRDNSKDT | MIYEVNKRPSGVPDRFSGSKSGDT | different VL |
| | LYLQMNSLRAEDTAVYYCASRSLLDY | ASLTVSGLQAEDEADYYCSSYAGS | |
| | WGQGTLVTVSS | PYVVFGGGTQLTVL | |
| | (SEQ ID NO: 9) | (SEQ ID NO: 95) | |
| 09C_ALCNK1_ | EVQLVESGGGLVQPGGSLRLSCAASG | QSALTQPPSASGSPGQSVTISCTG | VH Germline |
| germ | FTFSSYAMSWVRQAPGKGLEWVSAIS | TSSDVGGYNYVSWYQRHPGKAPKL | variant of |
| | GSGGSTYYADSVKGRFTISRDNSKNT | MIYEVNKRPSGVPDRFSGSKSGDT | 09C_ALCNK1 |
| | LYLQMNSLRAEDTAVYYCASRSLLDY | ASLTVSGLQAEDEADYYCSSYAGS | |
| | WGQGTLVTVSS | PYVVFGGGTQLTVL | |
| | (SEQ ID NO: 3) | (SEQ ID NO: 95) | |
| 01F_ALCNK1 | QVQLVESGGGLVQPGGSLRLSCAASG | QSALTQPASVSGSPGQSITISCTG | Same VH as H3 |
| | FTFSSYAMSWVRQAPGKGLEWVSAIS | TSNDVGNFNYVSWYQQHPGKAPKL | but a |
| | GSGGSTYYADSVKGRFTISRDNSKDT | MIYDVTNRPSGVPDRFSGSKSGNT | different VL |
| | LYLQMNSLRAEDTAVYYCASRSLLDY | ASLTISGLQAEDEAHYYCNSYTNS | |
| | WGQGTLVTVSS | DALILFGTGTKLTVL | |
| | (SEQ ID NO: 33) | (SEQ ID NO: 34) | |
| 01F_ALCNK1_ | EVQLVESGGGLVQPGGSLRLSCAASG | QSALTQPASVSGSPGQSITISCTG | VH Germline |
| germ | FTFSSYAMSWVRQAPGKGLEWVSAIS | TSNDVGNFNYVSWYQQHPGKAPKL | variant of |
| | GSGGSTYYADSVKGRFTISRDNSKNT | MIYDVTNRPSGVPDRFSGSKSGNT | 01F_ALCNK1 |
| | LYLQMNSLRAEDTAVYYCASRSLLDY | ASLTISGLQAEDEAHYYCNSYTNS | |
| | WGQGTLVTVSS | DALILFGTGTKLTVL | |
| | (SEQ ID NO: 3) | (SEQ ID NO: 34) | |
| 06A_ALCNK2 | QVQLVESGGGLVQPGGSLRLSCAASG | QSALTQPPSASGSPGQSVTISCTG | Same VH as H3 |
| | FTFSSYAMSWVRQAPGKGLEWVSAIS | SSSDLGRYDYVSWYQQHPGKGPKL | but a |
| | GSGGSTYYADSVKGRFTISRDNSKDT | MIYARNKRPSGVPDRFSGSKSGST | different VL |
| | LYLQMNSLRAEDTAVYYCASRSLLDY | ASLTIAGLQAEDEAVYYCSSYAGS | |
| | WGQGTLVTVSS | NNFVVFGAGTQLTVL | |
| | (SEQ ID NO: 35) | (SEQ ID NO: 36) | |
| 06A_ALCNK2_ | EVQLVESGGGLVQPGGSLRLSCAASG | QSALTQPPSASGSPGQSVTISCTG | VH Germline |
| germ | FTFSSYAMSWVRQAPGKGLEWVSAIS | SSSDLGRYDYVSWYQQHPGKGPKL | variant of |
| | GSGGSTYYADSVKGRFTISRDNSKNT | MIYARNKRPSGVPDRFSGSKSGST | 06A_ALCNK2 |
| | LYLQMNSLRAEDTAVYYCASRSLLDY | ASLTIAGLQAEDEAVYYCSSYAGS | |
| | WGQGTLVTVSS | NNFVVFGAGTQLTVL | |
| | (SEQ ID NO: 37) | (SEQ ID NO: 38) | |
| 04H_ALCNK1 | QVQLVESGGGLVQPGGSLRLSCAASG | NFMLTQDPAVSVAMGQTVTITCQG | Same VH as H3 |
| | FTFSSYAMSWVRQAPGKGLEWVSAIS | DSLRRYYASWYQKRPGQAPLLVFY | but a |
| | GSGGSTYYADSVKGRFTISRDNSKDT | GSNSRPSGVPDRISASFTWDKASL | different VL |
| | LYLQMNSLRAEDTAVYYCASRSLLDY | TITGAQAEDEADYYCSSMSGDLVV | |
| | WGQGTLVTVSS | FGGGTKLTVL | |
| | (SEQ ID NO: 39) | (SEQ ID NO: 40) | |
| 04H_ALCNK1_ | EVQLVESGGGLVQPGGSLRLSCAASG | NFMLTQDPAVSVAMGQTVTITCQG | VH Germline |
| germ | FTFSSYAMSWVRQAPGKGLEWVSAIS | DSLRRYYASWYQKRPGQAPLLVFY | variant of |
| | GSGGSTYYADSVKGRFTISRDNSKNT | GSNSRPSGVPDRISASFTWDKASL | 04H_ALCNK1 |
| | LYLQMNSLRAEDTAVYYCASRSLLDY | TITGAQAEDEADYYCSSMSGDLVV | |
| | WGQGTLVTVSS | FGGGTKLTVL | |
| | (SEQ ID NO: 41) | (SEQ ID NO: 42) | |
| 10C_ALCNK2 | QVQLVESGGGLVQPGGSLRLSCAASG | NFMLTQPPSLSVPPGQTASISCTA | Same VH as H3 |
| | FTFSSYAMSWVRQAPGKGLEWVSAIS | DRLGSRYVSWYQKKPGQSPVLIIY | but a |
| | GSGGSTYYADSVKGRFTISRDNSKDT | QDSKRPSVIPERFSGSNSGHTATL | different VL |
| | LYLQMNSLRAEDTAVYYCASRSLLDY | TISGTQPVDEADYFCQTWDSGTVA | |
| | WGQGTLVTVSS | FGGGTQLTVL | |
| | (SEQ ID NO: 39) | (SEQ ID NO: 44) | |
| 10C_ALCNK2_ | EVQLVESGGGLVQPGGSLRLSCAASG | NFMLTQPPSLSVPPGQTASISCTA | VH Germline |
| germ | FTFSSYAMSWVRQAPGKGLEWVSAIS | DRLGSRYVSWYQKKPGQSPVLIIY | variant of |
| | GSGGSTYYADSVKGRFTISRDNSKNT | QDSKRPSVIPERFSGSNSGHTATL | 10C_ALCNK2 |
| | LYLQMNSLRAEDTAVYYCASRSLLDY | TISGTQPVDEADYFCQTWDSGTVA | |
| | WGQGTLVTVSS | FGGGTQLTVL | |
| | (SEQ ID NO: 43) | (SEQ ID NO: 44) | |
| 01A_ALCNK2 | QVQLVESGGGLVQPGGSLRLSCAASG | RQSALTQPRSVSGSPGQSVAISCS | Same VH as H3 |
| | FTFSSYAMSWVRQAPGKGLEWVSAIS | GDNTEIYNYVAWYQQLPGQAPKLL | but a |
| | GSGGSTYYADSVKGRFTISRDNSKDT | IYDATERPSGVPDRFSGSKSGTTA | different VL |
| | LYLQMNSLRAEDTAVYYCASRSLLDY | SLTISGLQAEDEADYYCFSHEGSF | |
| | WGQGTLVTVSS | SGVFGGGTQLTVL | |
| | (SEQ ID NO: 45) | (SEQ ID NO: 46) | |
| 01A_ALCNK2_ | EVQLVESGGGLVQPGGSLRLSCAASG | RQSALTQPRSVSGSPGQSVAISCS | VH Germline |
| germ | FTFSSYAMSWVRQAPGKGLEWVSAIS | GDNTEIYNYVAWYQQLPGQAPKLL | variant of |
| | GSGGSTYYADSVKGRFTISRDNSKNT | IYDATERPSGVPDRFSGSKSGTTA | 01A_ALCNK2 |
| | LYLQMNSLRAEDTAVYYCASRSLLDY | SLTISGLQAEDEADYYCFSHEGSF | |
| | WGQGTLVTVSS | SGVFGGGTQLTVL | |
| | (SEQ ID NO: 47) | (SEQ ID NO: 48) | |
| 11D_ALCNK2 | QVQLVESGGGLVQPGGSLRLSCAASG | QSALTQPRSVSGSPGQSVTISCTG | Same VH as H3 |
| | FTFSSYAMSWVRQAPGKGLEWVSAIS | TYSDVGGYRYVSWYQQHPDKAPKL | but a |
| | GSGGSTYYADSVKGRFTISRDNSKDT | IIYDVDTRPSGVPDRFSGSKSGNT | different VL |
| | LYLQMNSLRAEDTAVYYCASRSLLDY | ASLTISGLQAEDDSDYYCFSYAGS | |
| | WGQGTLVTVSS | LTGVFGGGTQLTVL | |
| | (SEQ ID NO: 49) | (SEQ ID NO: 50) | |
| 11D_ALCNK2_ | EVQLVESGGGLVQPGGSLRLSCAASG | QSALTQPRSVSGSPGQSVTISCTG | VH Germline |
| germ | FTFSSYAMSWVRQAPGKGLEWVSAIS | TYSDVGGYRYVSWYQQHPDKAPKL | variant of |
| | GSGGSTYYADSVKGRFTISRDNSKNT | IIYDVDTRPSGVPDRFSGSKSGNT | 11D_ALCNK2 |
| | LYLQMNSLRAEDTAVYYCASRSLLDY | ASLTISGLQAEDDSDYYCFSYAGS | |
| | WGQGTLVTVSS | LTGVFGGGTQLTVL | |
| | (SEQ ID NO: 3) | (SEQ ID NO: 50) | |
| 05B_ALCNK1 | QVQLVESGGGLVQPGGSLRLSCAASG | QSALTQPASVSGSPGQSITISCTG | Same VH as H3 |
| | FTFSSYAMSWVRQAPGKGLEWVSAIS | TSSDLGNYNLVSWYQQHPGRAPKL | but a |
| | GSGGSTYYADSVKGRFTISRDNSKDT | LIYEVTKRPSGVSDRFSGSKSGNT | different VL |
| | LYLQMNSLRAEDTAVYYCASRSLLDY | ASLAISGLQAEDEGDYYCASYAGS | |
| | WGQGTLVTVSS | DFVIFGGGTKLTVL | |
| | (SEQ ID NO: 51) | (SEQ ID NO: 52) | |
| 05B_ALCNK1_ | EVQLVESGGGLVQPGGSLRLSCAASG | QSALTQPASVSGSPGQSITISCTG | VH Germline |
| germ | FTFSSYAMSWVRQAPGKGLEWVSAIS | TSSDLGNYNLVSWYQQHPGRAPKL | variant of |
| | GSGGSTYYADSVKGRFTISRDNSKNT | LIYEVTKRPSGVSDRFSGSKSGNT | 05B_ALCNK1 |
| | LYLQMNSLRAEDTAVYYCASRSLLDY | ASLAISGLQAEDEGDYYCASYAGS | |
| | WGQGTLVTVSS | DFVIFGGGTKLTVL | |
| | (SEQ ID NO: 53) | (SEQ ID NO: 54) | |
| 06B_ALCNK1 | QVQLVESGGGLVQPGGSLRLSCAASG | QSALTQPPSASGSPGQSVTISCTG | Same VH as H3 |
| | FTFSSYAMSWVRQAPGKGLEWVSAIS | TSSDLGRYNYVSWYQQHPGKAPKL | but a |
| | GSGGSTYYADSVKGRFTISRDNSKDT | IIHEVTKRPSGVPDRFSGSKSGNT | different VL |
| | LYLQMNSLRAEDTAVYYCASRSLLDY | ASLTVSGLQAEDEADYYCSSYAGN | |
| | WGQGTLVTVSS | YNWVFGGGTKLTVL | |
| | (SEQ ID NO: 55) | (SEQ ID NO: 56) | |
| 06B_ALCNK1_ | EVQLVESGGGLVQPGGSLRLSCAASG | QSALTQPPSASGSPGQSVTISCTG | VH Germline |
| germ | FTFSSYAMSWVRQAPGKGLEWVSAIS | TSSDLGRYNYVSWYQQHPGKAPKL | variant of |
| | GSGGSTYYADSVKGRFTISRDNSKNT | IIHEVTKRPSGVPDRFSGSKSGNT | 06B_ALCNK1 |
| | LYLQMNSLRAEDTAVYYCASRSLLDY | ASLTVSGLQAEDEADYYCSSYAGN | |
| | WGQGTLVTVSS | YNWVFGGGTKLTVL | |
| | (SEQ ID NO: 57) | (SEQ ID NO: 58) | |
| 12A_ALCNK2 | QVQLVESGGGLVQPGGSLRLSCAASG | QSALTQPPSASGSPGQSVTITCTG | Same VH as H3 |
| | FTFSSYAMSWVRQAPGKGLEWVSAIS | TSSDIGHYNYVSWYHQVPGKAPTL | but a |
| | GSGGSTYYADSVKGRFTISRDNSKDT | LISQVTERPSGVPDRFSGSKSGNT | different VL |
| | LYLQMNSLRAEDTAVYYCASRSLLDY | ASLTVSGLRTEDEGDYYCSSYVGN | |
| | WGQGTLVTVSS | NNYVFGRGTQLTVL | |
| | (SEQ ID NO: 59) | (SEQ ID NO: 60) | |
| 12A_ALCNK2_ | EVQLVESGGGLVQPGGSLRLSCAASG | QSALTQPPSASGSPGQSVTITCTG | VH Germline |
| germ | FTFSSYAMSWVRQAPGKGLEWVSAIS | TSSDIGHYNYVSWYHQVPGKAPTL | variant of |
| | GSGGSTYYADSVKGRFTISRDNSKNT | LISQVTERPSGVPDRFSGSKSGNT | 12A_ALCNK2 |
| | LYLQMNSLRAEDTAVYYCASRSLLDY | ASLTVSGLRTEDEGDYYCSSYVGN | |
| | WGQGTLVTVSS | NNYVFGRGTQLTVL | |
| | (SEQ ID NO: 61) | (SEQ ID NO: 62) | |
| 03A_ALCNK2 | QVQLVESGGGLVQPGGSLRLSCAASG | QSALTQPRSVSGSPGQSVTISCTG | Same VH as H3 |
| | FTFSSYAMSWVRQAPGKGLEWVSAIS | TSGDIGGFNYVSWYRHHPGRAPQL | but a |
| | GSGGSTYYADSVKGRFTISRDNSKDT | LIYDVDKRPSGVPDRFSGSKSGNT | different VL |
| | LYLQMNSLRAEDTAVYYCASRSLLDY | ASLSVSGLQSEDESDYYCYSYAGN | |
| | WGQGTLVTVSS | YHGLFGGGTKLTVL | |
| | (SEQ ID NO: 63) | (SEQ ID NO: 64) | |
| 03A_ALCNK2_ | EVQLVESGGGLVQPGGSLRLSCAASG | QSALTQPRSVSGSPGQSVTISCTG | VH Germline |
| germ | FTFSSYAMSWVRQAPGKGLEWVSAIS | TSGDIGGFNYVSWYRHHPGRAPQL | variant of |
| | GSGGSTYYADSVKGRFTISRDNSKNT | LIYDVDKRPSGVPDRFSGSKSGNT | 03A_ALCNK2 |
| | LYLQMNSLRAEDTAVYYCASRSLLDY | ASLSVSGLQSEDESDYYCYSYAGN | |
| | WGQGTLVTVSS | YHGLFGGGTKLTVL | |
| | (SEQ ID NO: 65) | (SEQ ID NO: 66) | |
| 06H_ALCNK2 | QVQLVESGGGLVQPGGSLRLSCAASG | VIWMTQSPSSVSASVGDRVTITCQ | Same VH as H3 |
| | FTFSSYAMSWVRQAPGKGLEWVSAIS | ASQDISSWLAWYQQQPGKAPKLLI | but a |
| | GSGGSTYYADSVKGRFTISRDNSKDT | YAASTLQTGVPSRFSGSGSGTNFS | different VL |
| | LYLQMNSLRAEDTAVYYCASRSLLDY | LTISSLQSEDFATYYCQQAKSFPS | |
| | WGQGTLVTVSS | ITFGQGTKREIK | |
| | (SEQ ID NO: 67) | (SEQ ID NO: 68) | |
| 06H_ALCNK2_ | EVQLVESGGGLVQPGGSLRLSCAASG | VIWMTQSPSSVSASVGDRVTITCQ | VH Germline |
| germ | FTFSSYAMSWVRQAPGKGLEWVSAIS | ASQDISSWLAWYQQQPGKAPKLLI | variant of |
| | GSGGSTYYADSVKGRFTISRDNSKNT | YAASTLQTGVPSRFSGSGSGTNFS | 06H_ALCNK2 |
| | LYLQMNSLRAEDTAVYYCASRSLLDY | LTISSLQSEDFATYYCQQAKSFPS | |
| | WGQGTLVTVSS | ITFGQGTKREIK | |
| | (SEQ ID NO: 69) | (SEQ ID NO: 70) | |
| 06H_ALCNK2_ | EVQLVESGGGLVQPGGSLRLSCAASG | DIQMTQSPSSVSASVGDRVTITCR | VH and VL |
| germVHVL | FTFSSYAMSWVRQAPGKGLEWVSAIS | ASQDISSWLAWYQQKPGKAPKLLI | Germline |
| | GSGGSTYYADSVKGRFTISRDNSKNT | YAASSLQSGVPSRFSGSGSGTDFT | variant of |
| | LYLQMNSLRAEDTAVYYCASRSLLDY | LTISSLQPEDFATYYCQQAKSFPS | 06H_ALCNK2 |
| | WGQGTLVTVSS | ITFGQGTKREIK | |
| | (SEQ ID NO: 71) | (SEQ ID NO: 72) | |
| 3F1 | EVQLVESGGGLVKPGGSLRLSCAASG | QSALTQPPSASGTPGQRVTISCSG | New ALCAM- |
| | FTLSDYSMNWVRQAPGKGLEWVASIS | SSSNIGSNSVSWYQQLPGTAPKLL | binding clone |
| | SRSSYIYYADSVKGRFTISRDNAKNS | IYSNTHRPSGVPDRFSGSKSGTSA | |
| | LYLGMNSLRAEDTAVYYCTGGQGYSG | SLAISGLLSEDEADYYCASWDDSL | |
| | YDGFQHWGQGTLVTVSS | NGGVFGGGTQLTVL | |
| | (SEQ ID NO: 73) | (SEQ ID NO: 74) | |
| 3F1germ | EVQLVESGGGLVKPGGSLRLSCAASG | QSVLTQPPSASGTPGQRVTISCSG | Similar to |
| | FTLSDYSMNWVRQAPGKGLEWVSSIS | SSSNIGSNSVNWYQQLPGTAPKLL | 3F1 with |
| | SRSSYIYYADSVKGRFTISRDNAKNS | IYSNTQRPSGVPDRFSGSKSGTSA | differences |
| | LYLQMNSLRAEDTAVYYCTGGQGYSG | SLAISGLQSEDEADYYCASWDDSL | in VH and VL |
| | YDGFQHWGQGTLVTVSS | NGGVFGGGTQLTVL | underlined |
| | (SEQ ID NO: 75) | (SEQ ID NO: 76) | |
| 3F1like_04G_ | EVQLVESGGGLVKPGGSLRLSCAASG | QSALTQPPSASGTPGQRVTISCSG | Same VH as |
| ALCNK2 | FTLSDYSMNWVRQAPGKGLEWVASIS | SSSNIGSNSVSWYQQLPGTAPKLL | 3F1 but a |
| | SRSSYIYYADSVKGRFTISRDNAKNS | IYSNTHRPSGVPDRFSGSKSGTSA | different VL |
| | LYLGMNSLRAEDTAVYYCTGGQGYSG | SLAISGLLSEDEADYYCASKDDSL | |
| | YDGFQHWGQGTLVTVSS | NGGVFGGGTQRTVL | |
| | (SEQ ID NO: 77) | (SEQ ID NO: 78) | |
| 3F1like_04G_ | EVQLVESGGGLVKPGGSLRLSCAASG | QSVLTQPPSASGTPGQRVTISCSG | Germline |
| ALCNK2_germ | FTLSDYSMNWVRQAPGKGLEWVSSIS | SSSNIGSNSVNWYQQLPGTAPKLL | variant of |
| | SRSSYIYYADSVKGRFTISRDNAKNS | IYSNTQRPSGVPDRFSGSKSGTSA | 04G_ALCNK2 |
| | LYLQMNSLRAEDTAVYYCTGGQGYSG | SLAISGLQSEDEADYYCASKDDSL | |
| | YDGFQHWGQGTLVTVSS | NGGVFGGGTQRTVL | |
| | (SEQ ID NO: 79) | (SEQ ID NO: 80) | |
| RYRgerm_ | EVQLVESGGGLVQPGGSLRLSCAASG | QSVLTQPPSVSGAPGQRVTISCTG | New EphA2- |
| 102919_15 | FTFSSYSMNWVRQAPGKGLEWVSYIS | SSSNIGAGYDVHWYQQLPGTAPKL | binder |
| | SSSSTIYYADSVKGRFTISRDNAKNS | LIYGNSNRPSGVPDRFSGSKSGTS | |
| | LYLQMNSLRAEDTAVYYCARYRLPNF | ASLAITGLQAEDEADYYCQSYDSS | |
| | WSGYPNYGTDVWGQGTTVTVSS | LSGHVVFGGGTKLTVL | |
| | (SEQ ID NO: 81) | (SEQ ID NO: 82) | |
| RYRgerm_ | EVQLVESGGGLVQPGGSLRLSCAASG | QSVLTQPPSVSGAPGQRVTISCTG | New EphA2- |
| 102919_14 | FTSSSYSMNWVRQAPGKGLEWVSYIS | SSSNIGAGYDVHWYQQLPGTAPKL | binder |
| | SSSSTIYYADSVKGRFTISRDNAKNS | LIYGNSNRPSGVPDRFSGSKSGTS | |
| | LYLQMNSLRAEDTAVYYCARYRLPNF | ASLAITGLQAEDEADYYCQSYDSS | |
| | WSGYPNYGMDVWGQGTTVTVSS | LSGHVVFGGGTKLTVL | |
| | (SEQ ID NO: 83) | (SEQ ID NO: 84) | |
| RYRgerm_ | EVQLVESGGGLVQPGGSLRLSCAASG | QSVLIQPPSVSGAPGQRVTISCIG | New EphA2- |
| 102919_22 | FTFSSYSMNWVRQAPGKGLEWVSYIS | SSSNIGAGYDVHWYQQLPGTAPKL | binder |
| | SSSSTIYYADSVKGRFTISRDNAKNS | LIYGNSNRPSGVPDRFSGSKSGTS | |
| | LYLQMNSLRAEDTAVYYCARYRLPNF | ASLAITGLQAEDEADYYCQSYDSS | |
| | WSGYPNYGMDVWGQGTTVTVSS | LSGHVVFGGGTKLTVL | |
| | (SEQ ID NO: 85) | (SEQ ID NO: 86) | |
| RYRgerm_ | EVQLVESGGGLVQPGGSLRLSCAASG | QSVLIQPPSVSGAPGQRVTISCIG | New EphA2- |
| 102919_33 | FTFSSYSMNWVRQAPGKGLEWVSYIS | SSSNIGAGYDVHWYQQLPGTAPKL | binder |
| | SSSSTIYYADSVKGRFTISRDNAKNS | LIYGNSNRPSGVPDRFSGSKSGTS | |
| | LYLQMNSLRAEDTAVYYCARYRFPDF | ASLAITGLQAEDEADYYCQSYDSS | |
| | WSGYPNYGMDVWGQGTTVTVSS | LSGHVVFGGGTKLTVL | |
| | (SEQ ID NO: 87) | (SEQ ID NO: 88) | |
| RYRgerm | EVQLVESGGGLVQPGGSLRLSCAASG | QSVLIQPPSVSGAPGQRVTISCIG | Original |
| | FTFSSYSMNWVRQAPGKGLEWVSYIS | SSSNIGAGYDVHWYQQLPGTAPKL | EphA2-binder |
| | SSSSTIYYADSVKGRFTISRDNAKNS | LIYGNSNRPSGVPDRFSGSKSGTS | |
| | LYLQMNSLRAEDTAVYYCARYRLPDF | ASLAITGLQAEDEADYYCQSYDSS | |
| | WSGYPNYGMDVWGQGTTVTVSS | LSGHVVFGGGTKLTVL | |
| | (SEQ ID NO: 89) | (SEQ ID NO: 90) | |
| RYR | QVQLQESGGGLVQPGGSLRLSCAASG | QSVLIQPPSVSGAPGQRVTISCIG | |
| | FTFSSYSMNWVRQAPGKGLEWVSYIS | SSSNIGAGYDVHWYQQLPGTAPKL | |
| | SSSSTIYYADSVKGRFTISRDNAKNS | LIYGNSNRPSGVPDRFSGSKSGTS | |
| | LYLQMNSLRAEDTAVYYCARYRLPDF | ASLAITGLQAEDEADYYCQSYDSS | |
| | WSGYPNYGMDVWGQGTTVTVSS | LSGHVVFGGGT | |
| | (SEQ ID NO: 96) | (SEQ ID NO: 97) | |

TABLE 5
Amino acid sequences of the HCDRs and LCDRs of exemplary engineered
antibodies in accordance to some non-limiting embodiments of the disclosure.

| Antibody | CDR1 | CDR2 | CDR3 |
|---|---|---|---|
| 3F1 VH | GFTLSDYS | ISSRSSYI | TGGQGYSGYDGFQH |
| | (SEQ ID NO: 98) | (SEQ ID NO: 99) | (SEQ ID NO: 100) |
| 3F1 VL | SSNIGSNS | SNT | ASWDDSLNGGV |
| | (SEQ ID NO: 101) | (SEQ ID NO: 102) | (SEQ ID NO: 103) |
| RYR VH | AASGFTFSSYSMN | YISSSSSTIY | ARYRLPDFWSGYPNYGMDV |
| | (SEQ ID NO: 104) | (SEQ ID NO: 105) | (SEQ ID NO: 106) |
| RYR VL | TGSSSNIGAGYDVH | YGNSNRPS | QSYDSSLSGHVV |
| | (SEQ ID NO: 107) | (SEQ ID NO: 108) | (SEQ ID NO: 109) |
| RYRgerm_102919_15 | AASGFTFSSYSMN | YISSSSSTIY | ARYRLPNFWSGYPNYGTDV |
| VH | (SEQ ID NO: 104) | (SEQ ID NO: 105) | (SEQ ID NO: 110) |
| RYRgerm_102919_15 | TGSSSNIGAGYDVH | YGNSNRPS | QSYDSSLSGHVV |
| VL | (SEQ ID NO: 107) | (SEQ ID NO: 108) | (SEQ ID NO: 109) |

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

1. de Goeij B E, Lambert J M. New developments for antibody-drug conjugate-based therapeutic approaches. Curr Opin Immunol 2016; 40:14-23 doi 10.1016/j.coi.2016.02.008.
2. Polakis P. Antibody Drug Conjugates for Cancer Therapy. Pharmacol Rev 2016; 68(1):3-19 doi 10.1124/pr.114.009373.
3. Gebleux R, Stringhini M, Casanova R, Soltermann A, Neri D. Non-internalizing antibody-drug conjugates display potent anti-cancer activity upon proteolytic release of monomethyl auristatin E in the subendothelial extracellular matrix. Int J Cancer 2017; 140(7):1670-9 doi 10.1002/ijc.30569.
4. Li J Y, Perry S R, Muniz-Medina V, Wang X, Wetzel L K, Rebelatto M C, et al. A Biparatopic HER2-Targeting Antibody-Drug Conjugate Induces Tumor Regression in Primary Models Refractory to or Ineligible for HER2-Targeted Therapy. Cancer Cell 2016; 29(1):117-29 doi 10.1016/j.ccell.2015.12.008.
5. de Goeij B E, Vink T, Ten Napel H, Breij E C, Satijn D, Wubbolts R, et al. Efficient Payload Delivery by a Bispecific Antibody-Drug Conjugate Targeting HER2 and CD63. Mol Cancer Ther 2016; 15(11):2688-97 doi 10.1158/1535-7163. MCT-16-0364.
6. Andreev J, Thambi N, Perez Bay A E, Delfino F, Martin J, Kelly M P, et al. Bispecific Antibodies and Antibody-Drug Conjugates (ADCs) Bridging HER2 and Prolactin Receptor Improve Efficacy of HER2 ADCs. Mol Cancer Ther 2017; 16(4):681-93 doi 10.1158/1535-7163. MCT-16-0658.
7. DeVay R M, Delaria K, Zhu G, Holz C, Foletti D, Sutton J, et al. Improved Lysosomal Trafficking Can Modulate the Potency of Antibody Drug Conjugates. Bioconjug Chem 2017; 28(4):1102-14 doi 10.1021/acs.bioconjchem.7b00013.
8. Lee N K, Zhang Y, Su Y, Bidlingmaier S, Sherbenou D W, Ha K D, et al. Cell-type specific potent Wnt signaling blockade by bispecific antibody. Sci Rep 2018; 8(1):766 doi 10.1038/s41598-017-17539-z.
9. Bruns C J, Harbison M T, Kuniyasu H, Eue I, Fidler I J. In vivo selection and characterization of metastatic variants from human pancreatic adenocarcinoma by using orthotopic implantation in nude mice. Neoplasia 1999; 1(1):50-62.
10. Su Y, Liu Y, Behrens C R, Bidlingmaier S, Lee N K, Aggarwal R, et al. Targeting CD46 for both adenocarcinoma and neuroendocrine prostate cancer. JCI Insight 2018; 3(17):pii: 121497 doi 10.1172/jci.insight.121497.
11. Lee N K, Bidlingmaier S, Su Y, Liu B. Modular Construction of Large Non-Immune Human Antibody Phage-Display Libraries from Variable Heavy and Light Chain Gene Cassettes. Methods Mol Biol 2018; 1701:61-82 doi 10.1007/978-1-4939-7447-4_4.
12. Rogosch T, Kerzel S, Hoi K H, Zhang Z, Maier R F, Ippolito G C, et al. Immunoglobulin analysis tool: a novel tool for the analysis of human and mouse heavy and light chain transcripts. Front Immunol 2012; 3:176 doi 10.3389/fimmu.2012.00176.
13. Smith K, Garman L, Wrammert J, Zheng N Y, Capra J D, Ahmed R, et al. Rapid generation of fully human monoclonal antibodies specific to a vaccinating antigen. Nat Protoc 2009; 4(3):372-84 doi 10.1038/nprot.2009.3.
14. Sherbenou D W, Aftab B T, Su Y, Behrens C R, Wiita A, Logan A C, et al. Antibody-drug conjugate targeting CD46 eliminates multiple myeloma cells. J Clin Invest 2016; 126(12):4640-53 doi 10.1172/JC185856.
15. Orcutt K D, Ackerman M E, Cieslewicz M, Quiroz E, Slusarczyk A L, Frangioni J V, et al. A modular IgG-scFv bispecific antibody topology. Protein Eng Des Sel 2010; 23(4):221-8 doi 10.1093/protein/gzp077.
16. Ha K D, Bidlingmaier S M, Zhang Y, Su Y, Liu B. High-content analysis of antibody phage-display library selection outputs identifies tumor selective macropinocytosis-dependent rapidly internalizing antibodies. Mol Cell Proteomics 2014; 13(12):3320-31 doi 10.1074/mcp.M114.039768.
17. Junutula J R, Raab H, Clark S, Bhakta S, Leipold D D, Weir S, et al. Site-specific conjugation of a cytotoxic drug to an antibody improves the therapeutic index. Nat Biotechnol 2008; 26(8):925-32 doi 10.1038/nbt.1480.
18. Jiao J, Hindoyan A, Wang S, Tran L M, Goldstein A S, Lawson D, et al. Identification of CD166 as a surface marker for enriching prostate stem/progenitor and cancer initiating cells. PLoS One 2012; 7(8):e42564 doi 10.1371/journal.pone.0042564.
19. Yan M, Yang X, Wang L, Clark D, Zuo H, Ye D, et al. Plasma membrane proteomics of tumor spheres identify CD166 as a novel marker for cancer stem-like cells in head and neck squamous cell carcinoma. Mol Cell Proteomics 2013; 12(11):3271-84 doi 10.1074/mcp.M112.025460.
20. Moon B S, Jeong W J, Park J, Kim T I, Min do S, Choi K Y. Role of oncogenic K-Ras in cancer stem cell activation by aberrant Wnt/beta-catenin signaling. J Natl Cancer Inst 2014; 106(2):djt373 doi 10.1093/jnci/djt373.
21. Behrens C R, Liu B. Methods for site-specific drug conjugation to antibodies. MAbs 2014; 6(1):46-53 doi 10.4161/mabs.26632.
22. Fu Y, Ho M. DNA damaging agent-based antibody-drug conjugates for cancer therapy. Antib Ther 2018; 1(2):33-43 doi 10.1093/abt/tby007.
23. Panowski S, Bhakta S, Raab H, Polakis P, Junutula J R. Site-specific antibody drug conjugates for cancer therapy. MAbs 2014; 6(1):34-45 doi 10.4161/mabs.27022.
24. Ha K D, Bidlingmaier S M, Su Y, Lee N K, Liu B. Identification of Novel Macropinocytosing Human Antibodies by Phage Display and High-Content Analysis. Methods Enzymol 2017; 585:91-110 doi 10.1016/bs.mie.2016.10.004.
25. Chang K, Pastan I. Molecular cloning of mesothelin, a differentiation antigen present on mesothelium, mesotheliomas, and ovarian cancers. Proc Natl Acad Sci USA 1996; 93(1):136-40.
26. Silver D A, Pellicer I, Fair W R, Heston W D, Cordon-Cardo C. Prostate-specific membrane antigen expression in normal and malignant human tissues. Clin Cancer Res 1997; 3(1):81-5.
27. Kinoshita Y, Kuratsukuri K, Landas S, Imaida K, Rovito P M, Jr., Wang C Y, et al. Expression of prostate-specific membrane antigen in normal and malignant human tissues. World J Surg 2006; 30(4):628-36 doi 10.1007/s00268-005-0544-5.
28. Wang K, Wei G, Liu D. CD19: a biomarker for B cell development, lymphoma diagnosis and therapy. Exp Hematol Oncol 2012; 1(1):36 doi 10.1186/2162-3619-1-36.
29. Vainshtein I, Roskos L K, Cheng J, Sleeman M A, Wang B, Liang M. Quantitative measurement of the target-mediated internalization kinetics of biopharmaceuticals. Pharm Res. 2015 January; 32(1):286-99.
30. Li N, Hill K S, Elferink L A. Analysis of receptor tyrosine kinase internalization using flow cytometry. Methods Mol Biol. 2008; 457:305-17.
31. Pahara J, Shi H, Chen X, Wang Z. Dimerization drives PDGF receptor endocytosis through a C-terminal hydrophobicmotif shared by EGF receptor. Exp Cell Res. 2010; 316:2237-50.
32. Mazor Y, Barnea I, Keydar I, Benhar I. Antibody internalization studied using a novel IgG binding toxin fusion. J Immunol Methods. 2007; 321:41-59.
33. Schaefer G et al. A two-in-one antibody against HER3 and EGFR has superior inhibitory activity compared with monospecific antibodies. Cancer Cell. 2011 Oct. 18; 20(4):472-86. doi: 10.1016/j.ccr.2011.09.003
34. Lee C V, Koenig P, Fuh G. A two-in-one antibody engineered from a humanized interleukin 4 antibody through mutation in heavy chain complementarity-determining regions. MAbs. 2014; 6(3):622-627. doi:10.4161/mabs.28483)

What is claimed is:

1. An engineered antibody or functional fragment thereof comprising:
a first antigen binding moiety capable of binding to a cell-surface guide antigen having a first rate of cellular internalization; and
a second antigen binding moiety capable of binding to a cell-surface effector antigen having a second rate of cellular internalization,
wherein the internalization property of the engineered antibody or functional fragment thereof is determined by a relative surface density ratio of the guide antigen to the effector antigen, and
wherein one of the two cellular internalization rates is at least 50%, at least 70%, at least 80%, or at least 90% greater than the other rate.

2. The engineered antibody or functional fragment thereof of claim 1, wherein the cell-surface guide antigen is an internalizing cell surface antigen.

3. The engineered antibody or functional fragment thereof of claim 1, wherein the cell-surface effector antigen is a non-internalizing cell surface antigen.

4. The engineered antibody or functional fragment thereof of any one of claims 1 to 2, wherein the relative surface density ratio of the guide antigen to the effector antigen is greater than a threshold value.

5. The engineered antibody or functional fragment thereof of any one of claims 1 to 2, wherein the relative surface density ratio of the guide antigen to the effector antigen is below a threshold value.

6. The engineered antibody or functional fragment thereof of any one of claims 1 to 5, wherein the threshold value is about 1:1, about 1:2, about 1:3, about 1:4, about 1:5, about 1:10, about 1:20, or about 1:30.

7. The engineered antibody or functional fragment thereof of any one of claims 1 to 6, wherein the first antigen binding moiety and the second antigen binding moiety are independently selected from the group consisting of an antigen-binding fragment (Fab), a single-chain variable fragment (scFv), a full-length immunoglobulin, a nanobody, a single domain antibody (sdAb), a VNAR domain, and a VHH domain, a multispecific antibody, a diabody, or a functional fragment thereof.

8. The engineered antibody or functional fragment thereof of any one of claims 1 to 7, wherein the guide antigen and the effector antigen are independently selected from the group consisting of activated leukocyte cell adhesion molecule (ALCAM), neural cell adhesion molecule (NCAM), calcium-activated chloride channel 2 (CaCC), carbonic anhydrase IX, carcinoembroyonic antigen (CEA), cathepsin G, CD19, CD20, CD22, CD30, CD33, CD38, CD44, CD44v6, CD46, CD52, CD71, CD73, CD272, CD276, B-cell maturation antigen (BCMA), epithelial cell adhesion molecule (EpCAM), ephrin type-A receptor 2 (EphA2), ephrin type-A receptor 3 (EphA3), ephrin type-A receptor 4 (EphA4), ephrin B2, receptor tyrosine kinase like orphan receptor 1 (ROR1), folate receptor, FLT3 (CD135), KIT (CD117), CD213A2, IL-1Ra, PRSS21, VEGFR2, CD24, PDGFR-beta, SSEA-4, epidermal growth factor receptor (EGFR), Erb-B2 receptor tyrosine kinase 2 (ErbB2), Erb-B2 receptor tyrosine kinase 3 (ErbB3), Erb-B2 receptor tyrosine kinase 4 (ErbB4), folate binding proteins (folate receptors), ganglioside, gangliosides, gp100, gpA33, immature laminin receptor, intercellular adhesion molecule 1 (ICAM-1), Lewis-Y, mesothelin, prostate stem cell antigen (PSCA), mucin 16 (MUC16 or CA-125), mucin 1 cell-surface associated (MUC1), mucin 2 oligomeric mucus gel-forming (MUC2), mucins, prostate membrane specific antigen (PSMA), TEM1/CD248, TEM7R, CLDN6, thyroid stimulating hormone receptor (TSHR), GPRC5D, CD97, CD179a, anaplastic lymphoma kinase (ALK or CD246), immunoglobulin lambda like polypeptide 1 (IGLL1), P-selectin, c-Met, fibroblast growth factor receptors (FGFRs), insulin-like growth factor 1 receptor (IGF-1R), tumor-associated calcium signal transducer 2 (Trop-2), and tumor associated glycoprotein 72 (TAG-72).

9. The engineered antibody or functional fragment thereof of any one of claims 1 to 8, wherein the guide antigen is a cancer-associated antigen selected from the group consisting of CD19, CD22, HER2 (ErbB2/neu), mesothelin, PSCA, CD123, CD30, CD71, CD171, CS-1, CLECL1, CD33, EGFRvIII, GD2, GD3, BCMA, PSMA, receptor tyrosine kinase like orphan receptor 1 (ROR1), folate receptor, FLT3 (CD135), TAG72, CD38, CD44v6, CD46, CEA, EpCAM, CD272, B7H3 (CD276), KIT (CD117), CD213A2, IL-1Ra, PRSS21, VEGFR2, CD24, PDGFR-beta, SSEA-4, CD20, MUC1, MUC16, EGFR, ErbB2, ErbB3, ErbB4, NCAM, prostatic acid phosphatase (PAP), ephrin B2, fibroblast activation protein (FAP), EphA2, c-Met, fibroblast growth factor receptors (FGFRs), insulin-like growth factor 1 receptor (IGF-1R), GM3, TEM1/CD248, TEM7R, CLDN6, thyroid stimulating hormone receptor (TSHR), GPRC5D, CD97, CD179a, anaplastic lymphoma kinase (ALK or CD246), and immunoglobulin lambda like polypeptide 1 (IGLL1).

10. The engineered antibody or functional fragment thereof of any one of claims 1 to 9, wherein the effector antigen is selected from the group consisting of ALCAM, EpCAM, folate binding proteins, PSMA, PSCA, mesothelin, CD19, CD20, CD22, CD30, CD33, CD38, CD44, CD46, ICAM-1, CD55, CD59, CD70, CD71, CD73, CD97, BCMA, CD272, CD276, MUC1, MUC16, NCAM, CD24, EphA2, EphA3, EphA4, Ephrin B2, CEA, c-Met, FGFRs, IGF-1R, VEGFRs, PDGFRs, Trop-2, TAG-72, P-selectin, EGFR, ErbB2, ErbB3, and ErbB4.

11. The engineered antibody or functional fragment thereof of any one of claims 1 to 10, wherein the antibody or functional fragment thereof is conjugated or covalently bound to at least one moiety-of-interest (MOI) selected from the group consisting of therapeutic moieties, diagnostic agents, and moieties that improve pharmacokinetics.

12. The engineered antibody or functional fragment thereof of claim 11, wherein the at least one MOI is selected from the group consisting of an anticancer agent, an anti-autoimmune disease agent, an anti-inflammatory agent, an anti-bacterial agent, an antimicrobial agent, an antibiotic, an anti-infectious disease agent, and an antiviral agent.

13. The engineered antibody or functional fragment thereof of claim 12, wherein the at least one MOI is selected from the group consisting of cytotoxic anti-cancer agents, DNA chelators, microtubule inhibitors, topoisomerase inhibitors, translation initiation inhibitors, ribosome inactivating molecules, nuclear transport inhibitors, RNA splicing inhibitors, RNA polymerase inhibitors, and DNA polymerase inhibitors.

14. The engineered antibody or functional fragment thereof of claim 13, wherein the cytotoxic anti-cancer agent is selected from the group consisting of auristatins, dolastatins, tubulysins, maytansinoids, taxanes, vinca alkaloids, amatoxins, anthracyclines, calicheamycins, camptothecins, irinotecan, SN-38, combretastatins, duocarmycins, enediynes, epothilones, ethylenimines, mytomycins, pyrrolobenzodiazepines (PBDs), and calicheamicin.

15. The engineered antibody or functional fragment thereof of any one of claims 11 to 14, wherein the at least one moiety-of-interest (MOI) is conjugated or covalently bound to a constant region of the engineered antibody or functional fragment thereof.

16. The engineered antibody or functional fragment thereof of claim 15, wherein the at least one moiety-of-interest (MOI) is conjugated or covalently bound to a heavy chain constant (CH1) region of the engineered antibody or functional fragment thereof.

17. The engineered antibody or functional fragment thereof of claim 15, wherein the at least one moiety-of-interest (MOI) is conjugated or covalently bound to a light chain constant (CL) region of the engineered antibody or functional fragment thereof.

18. The engineered antibody or functional fragment thereof of any one of claims 11 to 17, wherein the mean number of MOIs per antibody (mean DAR) ranges from 1 to 20.

19. The engineered antibody or functional fragment thereof of claim 18, wherein the mean DAR is about 1 to about 5, about 2 to about 6, about 3 to about 7, about 3 to about 8, about 4 to about 9, about 5 to about 10, about 10 to about 15, about 15 to about 20, or about 10 to about 20.

20. The engineered antibody or functional fragment thereof of any one of claims 1 to 19, comprising:
a first antigen binding moiety capable of binding to an ephrin receptor A2 (EphA2) expressed on the surface of a cell; and
a second antigen binding moiety capable of binding to an activated leukocyte cell adhesion molecule (ALCAM) expressed on the surface of the same cell.

21. The engineered antibody or functional fragment thereof of claim 20, wherein surface density ratio of EphA2 to ALCAM is greater than a threshold value of about 1:5.

22. The engineered antibody or functional fragment thereof of any one of claims 1 to 21, wherein the engineered antibody or functional fragment thereof comprises an amino acid sequence having at least 80% sequence identity to any one of the amino acid sequences identified in Table 4.

23. The engineered antibody or functional fragment thereof of claim 22, wherein the first antigen binding moiety comprises a heavy chain variable (VH) region having at least 80% sequence identity to a VH sequence identified in Table 4.

24. The engineered antibody or functional fragment thereof of claim 23, wherein the first antigen binding moiety comprises a VH region having at least 80% sequence identity to SEQ ID NO: 81 or SEQ ID NO: 96.

25. The engineered antibody or functional fragment thereof of any one of claims 22 to 24, wherein the VH region of the first antigen binding moiety comprises three complementary determining regions HCDR1, HCDR2, and HCDR3 as identified in the Sequence Listing.

26. The engineered antibody or functional fragment thereof of claim 25, wherein the VH region of the first antigen binding moiety comprises HCDR1, HCDR2, and HCDR3 comprising:
(a) SEQ ID NO: 104, SEQ ID NO: 105, and SEQ ID NO: 106, respectively; or
(b) SEQ ID NO: 104, SEQ ID NO: 105, and SEQ ID NO: 110, respectively.

27. The engineered antibody or functional fragment thereof of any one of claims 22 to 26, wherein the first antigen binding moiety comprises a light chain variable (VL) region having at least 80% sequence identity to a VL sequence identified in Table 4.

28. The engineered antibody or functional fragment thereof of claim 27, wherein the first antigen binding moiety comprises a VL region having at least 80% sequence identity to SEQ ID NO: 82 or SEQ ID NO: 97.

29. The engineered antibody or functional fragment thereof of any one of claims 22 to 28, wherein the VL region of the first antigen binding moiety comprises CDRs as identified in the Sequence Listing.

30. The engineered antibody or functional fragment thereof of claim 29, wherein the VL region of the first antigen binding moiety comprises LCDR1, LCDR2, and LCDR3 comprising SEQ ID NO: 107, SEQ ID NO: 108, and SEQ ID NO: 109, respectively.

31. The engineered antibody or functional fragment thereof of any one of claims 22 to 30, wherein the second antigen binding moiety comprises a VH region having at least 80% sequence identity to a VH sequence identified in Table 4.

32. The engineered antibody or functional fragment thereof of claim 31, wherein the second antigen binding moiety comprises a VH region having at least 80% sequence identity to SEQ ID NO: 73 or SEQ ID NO: 75.

33. The engineered antibody or functional fragment thereof of any one of claims 22 to 32, wherein the VH region of the second antigen binding moiety comprises three HCDRs as identified in the Sequence Listing.

34. The engineered antibody or functional fragment thereof of claim 33, wherein the VH region of the second antigen binding moiety comprises HCDR1, HCDR2, and HCDR3 comprising SEQ ID NO: 98, SEQ ID NO: 99, and SEQ ID NO: 100, respectively.

35. The engineered antibody or functional fragment thereof of any one of claims 22 to 34, wherein the second antigen binding moiety comprises a VL region having at least 80% sequence identity to a VL sequence identified in Table 4.

36. The engineered antibody or functional fragment thereof of claim 35, wherein the second antigen binding moiety comprises a VL region having at least 80% sequence identity to SEC ID NO: 74 or SEQ ID NO: 76.

37. The engineered antibody or functional fragment thereof of any one of claims 22 to 36, wherein the VL region of the second antigen binding moiety comprises three CDRs as identified in the Sequence Listing.

38. The engineered antibody or functional fragment thereof of claim 37, wherein the VL region of the second antigen binding moiety comprises LCDR1, LCDR2, and LCDR3 comprising SEQ ID NO: 101, SEQ ID NO: 102, and SEQ ID NO: 103, respectively.

39. A recombinant nucleic acid molecule comprising a nucleic acid sequence that encodes an engineered antibody or functional fragment thereof according to any one of claims 1 to 38.

40. The recombinant nucleic acid molecule of claim 39, wherein the recombinant nucleic acid molecule is operably linked to a heterologous nucleic acid sequence.

41. The recombinant nucleic acid molecule of any one of claims 39 to 40, wherein the recombinant nucleic acid molecule is further defined as an expression cassette or a vector.

42. A recombinant cell comprising:
an engineered antibody or functional fragment thereof according to any one of claims 1 to 36; and/or
a nucleic acid molecule according to any one of claims 39 to 41;

43. The recombinant cell of claim 42, wherein the recombinant cell is a prokaryotic cell or a eukaryotic cell.

44. A cell culture comprising at least one recombinant cell according to any one of claims 42 to 43 and a culture medium.

45. A pharmaceutical composition comprising one or more of the following:
an engineered antibody or functional fragment thereof according to any one of claims 1 to 38;
a nucleic acid molecule according to any one of claims 39 to 41; and
a recombinant cell according to any one of claims 42 to 43,
and a pharmaceutically acceptable carrier.

46. A method for modulating cellular internalization, comprising administering to a cell one or more of the following:
an engineered antibody or functional fragment thereof according to any one of claims 1 to 38;
a nucleic acid molecule according to any one of claims 39 to 41; and
a pharmaceutical composition according to claim 45.

47. A method for modulating cellular internalization, the method comprises administering to a cell an engineered antibody or functional fragment thereof comprising:
a first antigen binding moiety capable of binding to a cell-surface guide antigen having a first rate of cellular internalization; and
a second antigen binding moiety capable of binding to a cell-surface effector antigen having a second rate of cellular internalization,
wherein the internalization property of the engineered antibody or functional fragment thereof is determined by a relative surface density ratio of the guide antigen to the effector antigen, and
wherein one of the two cellular internalization rates is at least 50%, at least 70%, at least 80%, or at least 90% greater than the other rate.

48. A method for modulating cell-type selective signaling in a subject, the method comprises administering to the subject an engineered antibody or functional fragment thereof comprising:
a first antigen binding moiety capable of binding to a cell-surface guide antigen, wherein the guide antigen is expressed in the subject in a cell-type selective manner and has a first rate of cellular internalization; and
a second antigen binding moiety capable of binding to a cell-surface effector antigen having a second rate of cellular internalization,
wherein the internalization property of the engineered antibody or functional fragment thereof is determined by a relative surface density ratio of the guide antigen to the effector antigen; and
wherein one of the two cellular internalization rates is at least 50%, at least 70%, at least 80%, or at least 90% greater than the other rate.

49. A method for treating a health condition or disease in a subject in need thereof, the method comprises administering to the subject a therapeutically effective amount of an engineered antibody or functional fragment thereof comprising:
a first antigen binding moiety capable of binding to a cell-surface guide antigen having a first rate of cellular internalization; and
a second antigen binding moiety capable of binding to a cell-surface effector antigen having a second rate of cellular internalization,
wherein the internalization property of the engineered antibody or functional fragment thereof is determined by a relative surface density ratio of the guide antigen to the effector antigen; and
wherein one of the two cellular internalization rates is at least 50%, at least 70%, at least 80%, or at least 90% greater than the other rate.

50. The method of claim 49, wherein the health condition or disease is a cancer.

51. A method for killing a cancer cell, the method comprises administering to said cell an engineered antibody or functional fragment thereof comprising:
a first antigen binding moiety capable of binding to a cell-surface guide antigen having a first rate of cellular internalization; and
a second antigen binding moiety capable of binding to a cell-surface effector antigen having a second rate of cellular internalization.

52. The method of any one of claims 50 to 51, wherein the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

53. The method of any one of claims 46 to 52, wherein the cell-surface guide antigen is an internalizing cell surface antigen.

54. The method of any one of claims 46 to 52, wherein the cell-surface effector antigen is a non-internalizing cell surface antigen.

55. The method of any one of claims 46 to 54, wherein the relative surface density ratio of the guide antigen to the effector antigen is greater than a threshold value.

56. The method of any one of claims 46 to 54, wherein the relative surface density ratio of the guide antigen to the effector antigen is below a threshold value.

57. The method of any one of claims 46 to 56, wherein the threshold value is about 1:1, about 1:2, about 1:3, about 1:4, about 1:5, about 1:10, about 1:20, or about 1:30.

58. The method of any one of claims 46 to 57, further comprising modulating cell surface density of the guide antigen and/or cell surface density of the effector antigen.

59. The method of any one of claims 46 to 58, wherein the internalization property of the engineered antibody or functional fragment thereof is converted from non-internalizing to internalizing.

60. The method of any one of claims 46 to 58, wherein the internalization property of the engineered antibody or functional fragment thereof is converted from internalizing to non-internalizing.

61. The method of any one of claims 46 to 60, wherein the first antigen binding moiety and the second antigen binding moiety are independently selected from the group consisting of an antigen-binding fragment (Fab), a single-chain variable fragment (scFv), a full-length immunoglobulin, a nanobody, a single domain antibody (sdAb), a VNAR domain, and a VHH domain, a multispecific antibody, a diabody, or a functional fragment thereof.

62. The method of any one of claims 46 to 61, wherein the expression of the guide antigen and/or the effector antigen is cell-type selective.

63. The method of any one of claims 46 to 62, wherein the guide antigen and the effector antigen are independently selected from the group consisting of activated leukocyte cell adhesion molecule (ALCAM), neural cell adhesion molecule (NCAM), calcium-activated chloride channel 2 (CaCC), carbonic anhydrase IX, carcinoembroyonic antigen (CEA), cathepsin G, CD19, CD20, CD22, CD30, CD33, CD38, CD44, CD44v6, CD46, CD52, CD71, CD73, CD272, CD276, B-cell maturation antigen (BCMA), epithelial cell adhesion molecule (EpCAM), ephrin type-A receptor 2 (EphA2), ephrin type-A receptor 3 (EphA3), ephrin type-A receptor 4 (EphA4), ephrin B2, receptor tyrosine kinase like orphan receptor 1 (ROR1), folate receptor, FLT3 (CD135), KIT (CD117), CD213A2, IL-1Ra, PRSS21, VEGFR2, CD24, PDGFR-beta, SSEA-4, epidermal growth factor receptor (EGFR), Erb-B2 receptor tyrosine kinase 2 (ErbB2), Erb-B2 receptor tyrosine kinase 3 (ErbB3), Erb-B2 receptor tyrosine kinase 4 (ErbB4), folate binding proteins (folate receptors), ganglioside, gangliosides, gp100, gpA33, immature laminin receptor, intercellular adhesion molecule 1 (ICAM-1), Lewis-Y, mesothelin, prostate stem cell antigen (PSCA), mucin 16 (MUC16 or CA-125), mucin 1 cell surface associated (MUC1), mucin 2 oligomeric mucus gel-forming (MUC2), mucins, prostate membrance specific antigen (PSMA), TEM1/CD248, TEM7R, CLDN6, thyroid stimulating hormone receptor (TSHR), GPRC5D, CD97, CD179a, anaplastic lymphoma kinase (ALK or CD246), immunoglobulin lambda like polypeptide 1 (IGLL1), P-selectin, c-Met, fibroblast growth factor receptors (FGFRs), insulin-like growth factor 1 receptor (IGF-1R), tumor-associated calcium signal transducer 2 (Trop-2), and tumor associated glycoprotein 72 (TAG-72).

64. The method of any one of claims 46 to 63, wherein the guide antigen is a cancer-associated antigen selected from the group consisting of CD19, CD22, HER2 (ErbB2/neu), mesothelin, PSCA, CD123, CD30, CD71, CD171, CS-1, CLECL1, CD33, EGFRvIII, GD2, GD3, BCMA, PSMA, receptor tyrosine kinase like orphan receptor 1 (ROR1), folate receptor, FLT3 (CD135), TAG72, CD38, CD44v6, CD46, CEA, EpCAM, CD272, B7H3 (CD276), KIT (CD117), CD213A2, IL-1Ra, PRSS21, VEGFR2, CD24, PDGFR-beta, SSEA-4, CD20, MUC1, MUC16, EGFR, ErbB2, ErbB3, ErbB4, NCAM, prostatic acid phosphatase (PAP), ephrin B2, fibroblast activation protein (FAP), EphA2, c-Met, fibroblast growth factor receptors (FGFRs), insulin-like growth factor 1 receptor (IGF-1R), GM3, TEM1/CD248, TEM7R, CLDN6, thyroid stimulating hormone receptor (TSHR), GPRC5D, CD97, CD179a, anaplastic lymphoma kinase (ALK or CD246), and immunoglobulin lambda like polypeptide 1 (IGLL1).

65. The method of any one of claims 46 to 64, wherein the effector antigen is selected from the group consisting of ALCAM, EpCAM, Folate binding proteins, PSMA, PSCA, mesothelin, CD19, CD20, CD22, CD30, CD33, CD38, CD44, CD46, ICAM-1, CD55, CD59, CD70, CD71, CD73, CD97, BCMA, CD272, CD276, MUC1, MUC16, NCAM, CD24, EphA2, EphA3, EphA4, Ephrin B2, CEA, c-Met, FGFRs, IGF-1R, VEGFRs, PDGFRs, Trop-2, TAG-72, P-selectin, EGFR, ErbB2, ErbB3, and ErbB4.

66. The method of any one of claims 46 to 65, wherein the antibody or functional fragment thereof is conjugated or covalently bound to at least one moiety-of-interest (MOI) selected from the group consisting of therapeutic moieties, diagnostic agents, and moieties that improve pharmacokinetics.

67. The method of claim 66, wherein the at least one MOI is selected from the group consisting of an anticancer agent, an anti-autoimmune disease agent, an anti-inflammatory agent, an anti-bacterial agent, an antimicrobial agent, an antibiotic, an anti-infectious disease agent, and an antiviral agent.

68. The method of claim 67, wherein the at least one MOI is selected from the group consisting of cytotoxic anti-cancer agents, DNA chelators, microtubule inhibitors, topoisomerase inhibitors, translation initiation inhibitors, ribosome inactivating molecules, nuclear transport inhibitors, RNA splicing inhibitors, RNA polymerase inhibitors, and DNA polymerase inhibitors.

69. The method of claim 68, wherein the cytotoxic anti-agent is selected from the group consisting of auristatins, dolastatins, tubulysins, maytansinoids, taxanes, vinca alkaloids, amatoxins, anthracyclines, calicheamycins, camptothecins, irinotecan, SN-38, combretastatins, duocarmycins, enediynes, epothilones, ethylenimines, mytomycins, pyrrolobenzodiazepines (PBDs), and calicheamicin.

70. The method of any one of claims 66 to 69, wherein the at least one moiety-of-interest (MOI) is conjugated or covalently bound to a constant region of the engineered antibody or functional fragment thereof.

71. The method of claim 70, wherein the at least one moiety-of-interest (MOI) is conjugated or covalently bound to a CH1 region of the engineered antibody or functional fragment thereof.

72. The method of claim 70, wherein the at least one moiety-of-interest (MOI) is conjugated or covalently bound to a CL region of the engineered antibody or functional fragment thereof.

73. The method of any one of claims 46 to 72, wherein the mean number of MOIs per antibody (DAR) ranges from 1 to 20.

74. The method of claim 73, wherein the mean DAR is about 1 to about 5, about 2 to about 6, about 3 to about 7, about 3 to about 8, about 4 to about 9, about 5 to about 10, about 10 to about 15, about 15 to about 20, or about 10 to about 20.

75. The method of any one of claims 46 to 74, comprising:
a first antigen binding moiety capable of binding to an ephrin receptor A2 (EphA2) expressed on the surface of a cell; and
a second antigen binding moiety capable of binding to an activated leukocyte cell adhesion molecule (ALCAM) expressed on the surface of the same cell.

76. The method of claim 75, wherein the surface density ratio of EphA2 to ALCAM is greater than a threshold value of about 1:5.

77. The method of any one of claims 46 to 76, wherein engineered antibody or functional fragment thereof comprises an amino acid sequence having at least 80% sequence identity to any one of the amino acid sequences identified in Table 4.

78. A method of killing a tumor cell in a subject, the method comprises administering to said tumor cell an engineered antibody or functional fragment thereof comprising:
a first antigen binding moiety capable of binding to an ephrin receptor A2 (EphA2) expressed on the surface of said tumor cell; and
a second antigen binding moiety capable of binding to an activated leukocyte cell adhesion molecule (ALCAM) expressed on the surface of the same tumor cell.

79. The method of claim 78, wherein the surface density ratio of EphA2 to ALCAM is greater than a threshold value of about 1:5.

80. The method of any one of claims 46 to 79, wherein the engineered antibody or functional fragment thereof comprises an amino acid sequence having at least 80% sequence identity to any one of the amino acid sequences identified in Table 4.

81. The method of claim 80, wherein the first antigen binding moiety comprises a VH region having at least 80% sequence identity to a VH sequence identified in Table 4.

82. The method of claim 81, wherein the first antigen binding moiety comprises a VH region having at least 80% sequence identity to SEQ ID NO: 81 or SEQ ID NO: 96.

83. The method of any one of claims 80 to 82, wherein the VH region of the first antigen binding moiety comprises three CDRs as identified in the Sequence Listing.

84. The method of claim 83, wherein the VH region of the first antigen binding moiety comprises HCDR1, HCDR2, and HCDR3 comprising:
SEQ ID NO: 104, SEQ ID NO: 105, and SEQ ID NO: 106, respectively; or
SEQ ID NO: 104, SEQ ID NO: 105, and SEQ ID NO: 110, respectively.

85. The method of any one of claims 80 to 84, wherein the first antigen binding moiety comprises a VL region having at least 80% sequence identity to a VL sequence identified in Table 4.

86. The method of claim 85, wherein the first antigen binding moiety comprises a VL region having at least 80% sequence identity to SEQ ID NO: 82 or SEQ ID NO: 97.

87. The method of any one of claims 80 to 86, wherein the VL region of the first antigen binding moiety comprises three CDRs as identified in the Sequence Listing.

88. The method of claim 87, wherein the VL region of the first antigen binding moiety comprises LCDR1, LCDR2, and LCDR3 comprising SEQ ID NO: 107, SEQ ID NO: 108, and SEQ ID NO: 109, respectively.

89. The method of any one of claims 80 to 88, wherein the second antigen binding moiety comprises a VH region having at least 80% sequence identity to a VH sequence identified in Table 4.

90. The method of claim 89, wherein the second antigen binding moiety comprises a VH region having at least 80% sequence identity to SEQ ID NO: 73 or SEQ ID NO: 75.

91. The method of any one of claims 80 to 90, wherein the VH region of the second antigen binding moiety comprises three CDRs as identified in the Sequence Listing.

92. The method of claim 91, wherein the VH region of the second antigen binding moiety comprises HCDR1, HCDR2, and HCDR3 comprising SEQ ID NO: 98, SEQ ID NO: 99, and SEQ ID NO: 100, respectively.

93. The method of any one of claims 80 to 92, wherein the second antigen binding moiety comprises a VL region having at least 80% sequence identity to a VL sequence identified in Table 4.

94. The method of claim 93, wherein the second antigen binding moiety comprises a VL region having at least 80% sequence identity to SEC ID NO: 74 or SEQ ID NO: 76.

95. The method of any one of claims 80 to 94, wherein the VL region of the second antigen binding moiety comprises three CDRs as identified in the Sequence Listing.

96. The method of claim 95, wherein the VL region of the second antigen binding moiety comprises LCDR1, LCDR2, and LCDR3 comprising SEQ ID NO: 101, SEQ ID NO: 102, and SEQ ID NO: 103, respectively.


SEQUENCE LISTING (110 sequences)

SEQ ID NO: 1 - protein, 115 aa, Artificial sequence
  Synthetic polypeptide
  misc_feature (26)..(33): HCDR1
  misc_feature (51)..(58): HCDR2
  misc_feature (97)..(104): HCDR3
QVQLVESGGGLVQPGGSLRLSCAASGFTFSSYAMSWVRQAPGKGLEWVSAISGSGGSTYYADSVKGRFTISRDNSKDTLYLQMNSLRAEDTAVYYCASRSLLDYWGQGTLVTVSS

SEQ ID NO: 2 - protein, 107 aa, Artificial sequence
  Synthetic polypeptide
  misc_feature (26)..(31): LCDR1
  misc_feature (49)..(51): LCDR2
  misc_feature (88)..(97): LCDR3
NFMLTQDPAVSVALGQTVRITCQGDSLRSYYASWYQQKPGQAPLLVIYGKNNRPSGIPDRFSGSSSGNTASLTITGAQAEDEADYYCNSRDSSGNPVFGGGTKVTVL

SEQ ID NO: 3 - protein, 115 aa, Artificial sequence
  Synthetic polypeptide
  misc_feature (26)..(33): HCDR1
  misc_feature (51)..(58): HCDR2
  misc_feature (97)..(104): HCDR3
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCASRSLLDYWGQGTLVTVSS

SEQ ID NO: 4 - protein, 107 aa, Artificial sequence
  Synthetic polypeptide
  misc_feature (26)..(31): LCDR1
  misc_feature (49)..(51): LCDR2
  misc_feature (88)..(97): LCDR3
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPVFGGGTKVTVL

SEQ ID NO: 5 - protein, 115 aa, Artificial sequence
  Synthetic polypeptide
  misc_feature (26)..(33): HCDR1
  misc_feature (51)..(58): HCDR2
  misc_feature (97)..(104): HCDR3
EVQLVESGGGLVQPGGSQRLSCAASGFTFSSYAMSWVRQAPGKGLEWVSAISGSGGSTYYADSVKGRFTISRDNSKDTLYLQMNSLRAEDTAVYYCASRSLLDYWGQGTLVTVSS

SEQ ID NO: 6 - protein, 107 aa, Artificial sequence
  Synthetic polypeptide
  misc_feature (26)..(31): LCDR1
  misc_feature (49)..(51): LCDR2
  misc_feature (88)..(97): LCDR3
QSALTQDPAVSVALGQTVRITCQGDSLRSYYASWYQQKPGQAPLLVIYGKNNRPSGIPDRFSGSSSGNTASLTITGAQAEDEADYYCNSRDSSGNPVFGGGTKLTVL

SEQ ID NO: 7 - protein, 115 aa, Artificial sequence
  Synthetic polypeptide
  misc_feature (26)..(33): HCDR1
  misc_feature (51)..(58): HCDR2
  misc_feature (97)..(104): HCDR3
EVQLVESGGGLVQPGGSLRLSCAASGFTFSSYAMSWVRQAPGKGLEWVSAISGSGGSTYYADSVKGRFTISRDNSKDTLYLQMNSLRAEDTAVYYCASRSLLDYWGQGTLVTVSS

SEQ ID NO: 8 - protein, 107 aa, Artificial sequence
  Synthetic polypeptide
  misc_feature (26)..(31): LCDR1
  misc_feature (49)..(51): LCDR2
  misc_feature (88)..(97): LCDR3
QSVLTQDPAVSVALGQTVRITCQGDSLRSYYASWYQQKPGQAPVLVIYGKNNRPSGIPDRFSGSSSGNTASLTITGAQAEDEADYYCNSRDSSGNHVFGGGTQLTVL

SEQ ID NO: 9 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 10 - protein, 107 aa, Artificial sequence
  Synthetic polypeptide
  misc_feature (26)..(31): LCDR1
  misc_feature (49)..(51): LCDR2
  misc_feature (88)..(97): LCDR3
NFMLTQDPAVSVALGQTVRITCQGDSLRSYYASWYQQKPGQAPVLVIYGKNNRPSGIPDRFSGSSSGNTASLTITGAQAEDEADYYCNSRDSSGNHLFGGGTKLTVL

SEQ ID NO: 11 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 12 - protein, 107 aa, Artificial sequence
  Synthetic polypeptide
  misc_feature (26)..(31): LCDR1
  misc_feature (49)..(51): LCDR2
  misc_feature (88)..(97): LCDR3
NFMLTQDPAVSVALGQTVRITCQGESLRRYYGSWYHQRPGQAPLLVFYGKNRRPSGIPDRFSGSSSGDTATLTITGAQAEDEGAFYCNSRDGSANHFFGGGTQLTVL

SEQ ID NO: 13 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 14 - protein, 110 aa, Artificial sequence
  Synthetic polypeptide
  misc_feature (26)..(34): LCDR1
  misc_feature (52)..(54): LCDR2
  misc_feature (91)..(100): LCDR3
QSALTQPASVSGSPGQSITISCTGTSTDIGYYNYVSWYQQHPGKAPKLMIYDVSKRPSGVSNRFSGSKSGNTASLTISGLQAEDEADYYCSSFTSSTTYVFGTGTQLTVL

SEQ ID NO: 15 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (91)..(100): HCDR3

SEQ ID NO: 16 - protein, 111 aa, Artificial sequence
  Synthetic polypeptide
  misc_feature (26)..(34): LCDR1
  misc_feature (52)..(54): LCDR2
  misc_feature (91)..(101): LCDR3
QSALTQPASMSGSPGQSITMSCTGTSSDVGGSNYVSWYQQHPGKAPKLIIYDVTNRPSGVSNRFSGSKSGNTASLTISGLQAEDEADYYCSSFTSSTTLVVFGGGTKITVL

SEQ ID NO: 17 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 18 - protein, 111 aa, Artificial sequence
  Synthetic polypeptide
  misc_feature (26)..(34): LCDR1
  misc_feature (52)..(54): LCDR2
  misc_feature (91)..(101): LCDR3
QSALTQPASVSGSPGQSITISCTGTSSDVGGYNYVSWYQQHPGKAPKLMIYDVSNRPSGVSNRFSGSKSGNTASLTISGLQAEDEADYYCSSFTSSSTLYVFGTGTQLTVL

SEQ ID NO: 19 - protein, 115 aa, Artificial sequence; identical to SEQ ID NO: 3
  Synthetic polypeptide
  misc_feature H(26)..(33): CDR1
  misc_feature (51)..(58): HCDR2
  misc_feature (97)..(104): HCDR3

SEQ ID NO: 20 - protein, 111 aa, Artificial sequence; identical to SEQ ID NO: 18
  Synthetic polypeptide
  misc_feature (26)..(34): LCDR1
  misc_feature (52)..(54): LCDR2
  misc_feature (91)..(101): LCDR3

SEQ ID NO: 21 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 22 - protein, 111 aa, Artificial sequence
  Synthetic polypeptide
  misc_feature (26)..(34): LCDR1
  misc_feature (52)..(54): LCDR2
  misc_feature (91)..(101): LCDR3
QSALTQPASVSGSPGQSITISCTGTSSDVGGYNYVSWHQQHPGKAPKLMIYDVSNRPSGVSNRFSGSKSGNTASLTISGLQAEDEADYYCSSYTSSSSLYVFGTGTQLTVL

SEQ ID NO: 23 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 24 - protein, 110 aa, Artificial sequence
  Synthetic polypeptide
  misc_feature (26)..(34): LCDR1
  misc_feature (52)..(54): LCDR2
  misc_feature (91)..(100): LCDR3
QSALTQPASVSGSPGQSVTISCTGTNSDVGAYNYVSWYQQHPGKAPKLMIYDVSNRPSGVSNRFSGSKSGNTASLTISGLQTEDEADYYCTSYTSSNTRVFGGGTQLTVL

SEQ ID NO: 25 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 26 - protein, 110 aa, Artificial sequence; identical to SEQ ID NO: 24
  Synthetic polypeptide
  misc_feature (26)..(34): LCDR1
  misc_feature (52)..(54): LCDR2
  misc_feature (91)..(100): LCDR3

SEQ ID NO: 27 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 28 - protein, 111 aa, Artificial sequence
  Synthetic polypeptide
  misc_feature (26)..(34): LCDR1
  misc_feature (52)..(54): LCDR2
  misc_feature (91)..(100): LCDR3
QSALTQPPSASGSPGQSVTISCTGTSSDVGRYNLVSWYQRHPGKVPKLIIYEVTKRPSGVPDRFSGSKSGNTASLTVSGLQAEDEADYYCSSHGSSNAFYVFGTGTKLTVL

SEQ ID NO: 29 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 30 - protein, 111 aa, Artificial sequence
  Synthetic polypeptide
  misc_feature (26)..(34): LCDR1
  misc_feature (52)..(54): LCDR2
  misc_feature (91)..(101): LCDR3
QSALTQPASVSGSPGQSITISCTGTSRDVGGYDYVSWYQQHPGKAPKFIIYDVNKRPSGVSNRFSGSKSGNTASLTISGLQPEDEADYICSSFTMYSTPVIFGGGTQLTVL

SEQ ID NO: 31 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 32 - protein, 111 aa, Artificial sequence; identical to SEQ ID NO: 30
  Synthetic polypeptide
  misc_feature (26)..(34): LCDR1
  misc_feature (52)..(54): LCDR2
  misc_feature (91)..(101): LCDR3

SEQ ID NO: 33 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 34 - protein, 111 aa, Artificial sequence
  Synthetic polypeptide
  misc_feature (26)..(34): LCDR1
  misc_feature (52)..(54): LCDR2
  misc_feature (91)..(101): LCDR3
QSALTQPASVSGSPGQSITISCTGTSNDVGNFNYVSWYQQHPGKAPKLMIYDVTNRPSGVPDRFSGSKSGNTASLTISGLQAEDEAHYYCNSYTNSDALILFGTGTKLTVL

SEQ ID NO: 35 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 36 - protein, 111 aa, Artificial sequence
  Synthetic polypeptide
  misc_feature (26)..(34): LCDR1
  misc_feature (52)..(54): LCDR2
  misc_feature (91)..(101): LCDR3
QSALTQPPSASGSPGQSVTISCTGSSSDLGRYDYVSWYQQHPGKGPKLMIYARNKRPSGVPDRFSGSKSGSTASLTIAGLQAEDEAVYYCSSYAGSNNFVVFGAGTQLTVL

SEQ ID NO: 37 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 38 - protein, 111 aa, Artificial sequence; identical to SEQ ID NO: 36
  Synthetic polypeptide
  misc_feature (26)..(34): LCDR1
  misc_feature (52)..(54): LCDR2
  misc_feature (91)..(101): LCDR3

SEQ ID NO: 39 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 40 - protein, 106 aa, Artificial sequence
  Synthetic polypeptide
  misc_feature (26)..(31): LCDR1
  misc_feature (49)..(51): LCDR2
  misc_feature (88)..(96): LCDR3
NFMLTQDPAVSVAMGQTVTITCQGDSLRRYYASWYQKRPGQAPLLVFYGSNSRPSGVPDRISASFTWDKASLTITGAQAEDEADYYCSSMSGDLVVFGGGTKLTVL

SEQ ID NO: 41 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 42 - protein, 106 aa, Artificial sequence; identical to SEQ ID NO: 40
  Synthetic polypeptide
  misc_feature (26)..(31): LCDR1
  misc_feature (49)..(51): LCDR2
  misc_feature (88)..(96): LCDR3

SEQ ID NO: 43 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 44 - protein, 106 aa, Artificial sequence
  Synthetic polypeptide
  misc_feature (26)..(31): LCDR1
  misc_feature (49)..(51): LCDR2
  misc_feature (88)..(96): LCDR3
NFMLTQPPSLSVPPGQTASISCTADRLGSRYVSWYQKKPGQSPVLIIYQDSKRPSVIPERFSGSNSGHTATLTISGTQPVDEADYFCQTWDSGTVAFGGGTQLTVL

SEQ ID NO: 45 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 46 - protein, 109 aa, Artificial sequence
  Synthetic polypeptide
  misc_feature (27)..(33): LCDR1
  misc_feature (51)..(53): LCDR2
  misc_feature (91)..(99): LCDR3
RQSALTQPRSVSGSPGQSVAISCSGDNTEIYNYVAWYQQLPGQAPKLLIYDATERPSGVPDRFSGSKSGTTASLTISGLQAEDEADYYCFSHEGSFSGVFGGGTQLTVL

SEQ ID NO: 47 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 48 - protein, 109 aa, Artificial sequence; identical to SEQ ID NO: 46
  Synthetic polypeptide
  misc_feature (27)..(33): LCDR1
  misc_feature (51)..(53): LCDR2
  misc_feature (91)..(99): LCDR3

SEQ ID NO: 49 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 50 - protein, 110 aa, Artificial sequence
  Synthetic polypeptide
  misc_feature (26)..(34): LCDR1
  misc_feature (52)..(54): LCDR2
  misc_feature (91)..(100): LCDR3
QSALTQPRSVSGSPGQSVTISCTGTYSDVGGYRYVSWYQQHPDKAPKLIIYDVDTRPSGVPDRFSGSKSGNTASLTISGLQAEDDSDYYCFSYAGSLTGVFGGGTQLTVL

SEQ ID NO: 51 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 52 - protein, 110 aa, Artificial sequence
  Synthetic polypeptide
  misc_feature (26)..(34): LCDR1
  misc_feature (52)..(54): LCDR2
  misc_feature (91)..(100): LCDR3
QSALTQPASVSGSPGQSITISCTGTSSDLGNYNLVSWYQQHPGRAPKLLIYEVTKRPSGVSDRFSGSKSGNTASLAISGLQAEDEGDYYCASYAGSDFVIFGGGTKLTVL

SEQ ID NO: 53 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 54 - protein, 110 aa, Artificial sequence; identical to SEQ ID NO: 52
  Synthetic polypeptide
  misc_feature (26)..(34): LCDR1
  misc_feature (52)..(54): LCDR2
  misc_feature (91)..(100): LCDR3

SEQ ID NO: 55 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 56 - protein, 110 aa, Artificial sequence
  Synthetic polypeptide
  misc_feature (26)..(34): LCDR1
  misc_feature (52)..(54): LCDR2
  misc_feature (91)..(100): LCDR3
QSALTQPPSASGSPGQSVTISCTGTSSDLGRYNYVSWYQQHPGKAPKLIIHEVTKRPSGVPDRFSGSKSGNTASLTVSGLQAEDEADYYCSSYAGNYNWVFGGGTKLTVL

SEQ ID NO: 57 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 58 - protein, 110 aa, Artificial sequence; identical to SEQ ID NO: 56
  Synthetic polypeptide
  misc_feature (26)..(34): LCDR1
  misc_feature (52)..(54): LCDR2
  misc_feature (91)..(100): LCDR3

SEQ ID NO: 59 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 60 - protein, 110 aa, Artificial sequence
  Synthetic polypeptide
  misc_feature (26)..(34): LCDR1
  misc_feature (52)..(54): LCDR2
  misc_feature (91)..(100): LCDR3
QSALTQPPSASGSPGQSVTITCTGTSSDIGHYNYVSWYHQVPGKAPTLLISQVTERPSGVPDRFSGSKSGNTASLTVSGLRTEDEGDYYCSSYVGNNNYVFGRGTQLTVL

SEQ ID NO: 61 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 62 - protein, 110 aa, Artificial sequence; identical to SEQ ID NO: 60
  Synthetic polypeptide
  misc_feature (26)..(34): LCDR1
  misc_feature (52)..(54): LCDR2
  misc_feature (91)..(100): LCDR3

SEQ ID NO: 63 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR3

SEQ ID NO: 64 - protein, 110 aa, Artificial sequence
  Synthetic polypeptide
  misc_feature (26)..(34): LCDR1
  misc_feature (52)..(54): LCDR2
  misc_feature (91)..(100): LCDR3
QSALTQPRSVSGSPGQSVTISCTGTSGDIGGFNYVSWYRHHPGRAPQLLIYDVDKRPSGVPDRFSGSKSGNTASLSVSGLQSEDESDYYCYSYAGNYHGLFGGGTKLTVL

SEQ ID NO: 65 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 66 - protein, 110 aa, Artificial sequence; identical to SEQ ID NO: 64
  Synthetic polypeptide
  misc_feature (26)..(34): LCDR1
  misc_feature (52)..(54): LCDR2
  misc_feature (91)..(100): LCDR3

SEQ ID NO: 67 - protein, 115 aa, Artificial sequence; identical to SEQ ID NO: 1
  Synthetic polypeptide
  misc_feature (26)..(33): HCDR1
  misc_feature (51)..(58): HCDR2
  misc_feature (97)..(104): HCDR2

SEQ ID NO: 68 - protein, 108 aa, Artificial sequence
  Synthetic polypeptide
  misc_feature (27)..(32): LCDR1
  misc_feature (50)..(52): LCDR2
  misc_feature (89)..(98): LCDR3
VIWMTQSPSSVSASVGDRVTITCQASQDISSWLAWYQQQPGKAPKLLIYAASTLQTGVPSRFSGSGSGTNFSLTISSLQSEDFATYYCQQAKSFPSITFGQGTKREIK

SEQ ID NO: 69 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 70 - protein, 108 aa, Artificial sequence; identical to SEQ ID NO: 68
  Synthetic polypeptide
  misc_feature (27)..(32): LCDR1
  misc_feature (50)..(52): LCDR2
  misc_feature (89)..(98): LCDR3

SEQ ID NO: 71 - protein, 115 aa, Artificial sequence; identical to SEQ ID NO: 3
  Synthetic polypeptide
  misc_feature (26)..(33): HCDR1
  misc_feature (51)..(58): HCDR2
  misc_feature (97)..(104): HCDR3

SEQ ID NO: 72 - protein, 108 aa, Artificial sequence
  Synthetic polypeptide
  misc_feature (27)..(32): LCDR1
  misc_feature (50)..(52): LCDR2
  misc_feature (89)..(98): LCDR3
DIQMTQSPSSVSASVGDRVTITCRASQDISSWLAWYQQKPGKAPKLLIYAASSLQSGVPSRFSGSGSGTDFTLTISSLQPEDFATYYCQQAKSFPSITFGQGTKREIK

SEQ ID NO: 73 - protein, 121 aa, Artificial sequence
  Synthetic polypeptide
  misc_feature (26)..(33): HCDR1
  misc_feature (51)..(58): HCDR2
  misc_feature (97)..(110): HCDR3
EVQLVESGGGLVKPGGSLRLSCAASGFTLSDYSMNWVRQAPGKGLEWVASISSRSSYIYYADSVKGRFTISRDNAKNSLYLGMNSLRAEDTAVYYCTGGQGYSGYDGFQHWGQGTLVTVSS

SEQ ID NO: 74 - protein, 110 aa, Artificial sequence
  Synthetic polypeptide
  misc_feature (26)..(33): LCDR1
  misc_feature (51)..(53): LCDR2
  misc_feature (90)..(100): LCDR3
QSALTQPPSASGTPGQRVTISCSGSSSNIGSNSVSWYQQLPGTAPKLLIYSNTHRPSGVPDRFSGSKSGTSASLAISGLLSEDEADYYCASWDDSLNGGVFGGGTQLTVL

SEQ ID NO: 75 - protein, 121 aa, Artificial sequence
  Synthetic polypeptide
  misc_feature (26)..(33): HCDR1
  misc_feature (51)..(58): HCDR2
  misc_feature (97)..(110): HCDR3
EVQLVESGGGLVKPGGSLRLSCAASGFTLSDYSMNWVRQAPGKGLEWVSSISSRSSYIYYADSVKGRFTISRDNAKNSLYLQMNSLRAEDTAVYYCTGGQGYSGYDGFQHWGQGTLVTVSS

SEQ ID NO: 76 - protein, 110 aa, Artificial sequence
  Synthetic polypeptide
  misc_feature (26)..(33): LCDR1
  misc_feature (51)..(53): LCDR2
  misc_feature (90)..(100): LCDR3
QSVLTQPPSASGTPGQRVTISCSGSSSNIGSNSVNWYQQLPGTAPKLLIYSNTQRPSGVPDRFSGSKSGTSASLAISGLQSEDEADYYCASWDDSLNGGVFGGGTQLTVL

SEQ ID NO: 77 - protein, 121 aa, Artificial sequence; identical to SEQ ID NO: 73
  Synthetic polypeptide
  misc_feature (26)..(33): HCDR1
  misc_feature (51)..(58): HCDR2
  misc_feature (97)..(110): HCDR3

SEQ ID NO: 78 - protein, 110 aa, Artificial sequence
  Synthetic polypeptide
  misc_feature (26)..(33): LCDR1
  misc_feature (51)..(53): LCDR2
  misc_feature (90)..(100): LCDR3
QSALTQPPSASGTPGQRVTISCSGSSSNIGSNSVSWYQQLPGTAPKLLIYSNTHRPSGVPDRFSGSKSGTSASLAISGLLSEDEADYYCASKDDSLNGGVFGGGTQRTVL

SEQ ID NO: 79 - protein, 121 aa, Artificial sequence; identical to SEQ ID NO: 75
  Synthetic polypeptide
  misc_feature (26)..(33): HCDR1
  misc_feature (51)..(58): HCDR2
  misc_feature (97)..(110): HCDR3

SEQ ID NO: 80 - protein, 110 aa, Artificial sequence
  Synthetic polypeptide
  misc_feature (26)..(33): LCDR1
  misc_feature (51)..(53): LCDR2
  misc_feature (90)..(100): LCDR3
QSVLTQPPSASGTPGQRVTISCSGSSSNIGSNSVNWYQQLPGTAPKLLIYSNTQRPSGVPDRFSGSKSGTSASLAISGLQSEDEADYYCASKDDSLNGGVFGGGTQRTVL

SEQ ID NO: 81 - protein, 126 aa, Artificial sequence
  Synthetic polypeptide
  misc_feature (23)..(35): HCDR1
  misc_feature (50)..(59): HCDR2
  misc_feature (97)..(115): HcDR3
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPNFWSGYPNYGTDVWGQGTTVTVSS

SEQ ID NO: 82 - protein, 112 aa, Artificial sequence
  Synthetic polypeptide
  misc_feature (23)..(36): LCDR1
  misc_feature (51)..(58): LCDR2
  misc_feature (91)..(102): LCDR3
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGHVVFGGGTKLTVL

SEQ ID NO: 83 - protein, 126 aa, Artificial sequence
  Synthetic polypeptide
  misc_feature (23)..(35): HCDR1
  misc_feature (50)..(59): HCDR2
  misc_feature (97)..(115): HCDR3
EVQLVESGGGLVQPGGSLRLSCAASGFTSSSYSMNWVRQAPGKGLEWVSYISSSSSTIYYADSVKGRFTISRDNAKNSLYLQMNSLRAEDTAVYYCARYRLPNFWSGYPNYGMDVWGQGTTVTVSS

SEQ ID NO: 84 - protein, 112 aa, Artificial sequence; identical to SEQ ID NO: 82
  Synthetic polypeptide
  misc_feature (23)..(36): LCDR1
  misc_feature (51)..(58): LCDR2
  misc_feature (91)..(102): LCDR3

SEQ ID NO: 85 - protein, 126 aa, Artificial sequence
  Synthetic polypeptide
  misc_feature (23)..(35): HCDR1
  misc_feature (50)..(59): HCDR2
  misc_feature (97)..(115): HCDR3
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPNFWSGYPNYGMDVWGQGTTVTVSS

SEQ ID NO: 86 - protein, 112 aa, Artificial sequence; identical to SEQ ID NO: 82
  Synthetic polypeptide
  misc_feature (23)..(36): LCDR1
  misc_feature (51)..(58): LCDR2
  misc_feature (91)..(102): LCDR3

SEQ ID NO: 87 - protein, 126 aa, Artificial sequence
  Synthetic polypeptide
  misc_feature (23)..(35): HCDR1
  misc_feature (50)..(59): HCDR2
  misc_feature (97)..(115): HCDR3
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARYRFPDFWSGYPNYGMDVWGQGTTVTVSS

SEQ ID NO: 88 - protein, 112 aa, Artificial sequence; identical to SEQ ID NO: 82
  Synthetic polypeptide
  misc_feature (23)..(36): LCDR1
  misc_feature (51)..(58): LCDR2
  misc_feature (91)..(102): LCDR3

SEQ ID NO: 89 - protein, 126 aa, Artificial sequence
  Synthetic polypeptide
  misc_feature (23)..(35): HCDR1
  misc_feature (50)..(59): HCDR2
  misc_feature (97)..(115): HCDR3
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPDFWSGYPNYGMDVWGQGTTVTVSS

SEQ ID NO: 90 - protein, 112 aa, Artificial sequence; identical to SEQ ID NO: 82
  Synthetic polypeptide
  misc_feature (23)..(36): LCDR1
  misc_feature (51)..(58): LCDR2
  misc_feature (91)..(102): LCDR3

SEQ ID NO: 91 - DNA, 759 bp, Artificial sequence
  Synthetic polynucleotide
  misc_feature: encodes the peptide sequence of RYRgerm_102919_15
gaggtgcagctggttgagtcgggaggaggtttggtacagcctggaggttccctgagactctcctgtgcagcctctggattcaccttctcgagctatagcatgaactgggtccgccaggctccaggtaagggactggagtgggtttcatacatttctagttcaagtagtaccatatactacgcagactctgtgaagggacgattcaccatctccagagacaatgccaagaactcactgtatctgcaaatgaacagcctgagagccgaggacacggctgtgtattactgtgcgaggtatcgattaccgaatttttggagtggttaccctaactacggtacggacgtctggggacaaggaacaacagtcacagtttcatcaggtggaggaggttcaggtggaggtggctctggaggtggtggatctcagtctgttttgacgcagccgccctcagtgtctggtgccccagggcagagggtcaccatctcctgcactggaagcagctccaacatcggtgcaggttatgatgtacactggtaccagcagcttccaggaacagcccccaaactcctcatctatggtaacagcaatcggccctcaggagttcctgaccgattctctggatccaagtctggtacctcagcctccctggccatcactggactccaggctgaggatgaggctgattattactgccagtcctatgacagcagcctgtccggacatgtggtattcggtggaggaaccaagctgaccgtccta

SEQ ID NO: 92 - DNA, 759 bp, Artificial sequence
  Synthetic polynucleotide
  misc_feature: encodes the peptide sequence of RYRgerm_102919_14
gaggtgcagctggttgagtcgggaggaggtttggtacagcctggaggttccctgagactctcctgtgcagcctctggattcacctcctcgagctatagcatgaactgggtccgccaggctccaggtaagggactggagtgggtttcatacatttctagttcaagtagtaccatatactacgcagactctgtgaagggacgattcaccatctccagagacaatgccaagaactcactgtatctgcaaatgaacagcctgagagccgaggacacggctgtgtattactgtgcgaggtatcgattaccgaatttttggagtggttaccctaactacggtatggacgtctggggacaaggaacaacagtcacagtttcatcaggtggaggaggttcaggtggaggtggctctggaggtggtggatctcagtctgttttgacgcagccgccctcagtgtctggtgccccagggcagagggtcaccatctcctgcactggaagcagctccaacatcggtgcaggttatgatgtacactggtaccagcagcttccaggaacagcccccaaactcctcatctatggtaacagcaatcggccctcaggagttcctgaccgattctctggatccaagtctggtacctcagcctccctggccatcactggactccaggctgaggatgaggctgattattactgccagtcctatgacagcagcctgtccggacatgtggtattcggtggaggaaccaagctgaccgtccta

SEQ ID NO: 93 - DNA, 759 bp, Artificial sequence
  Synthetic polynucleotide
  misc_feature: encodes the peptide sequence of RYRgerm_102919_22
gaggtgcagctggtggagtctggtggaggattggtacagcctggaggttccctgagactctcctgcgcagcctctggattcaccttctcgagctatagcatgaactgggtccgccaggctccaggtaagggactggagtgggtttcatacatttctagttcaagtagtaccatatactacgcagactctgtgaagggacgattcaccatctccagagacaatgccaagaactcactgtatctgcaaatgaacagcctgagagccgaggacacggctgtgtattactgtgcgaggtatcgattaccgaatttttggagtggttaccctaactacggtatggacgtctggggacaaggaacaacagtcacagtttcatcaggtggaggaggttcaggtggaggtggctctggaggtggtggatctcagtctgttttgacgcagccgccctcagtgtctggtgccccagggcagagggtcaccatctcctgcactggaagcagctccaacatcggtgcaggttatgatgtacactggtaccagcagcttccaggaacagcccccaaactcctcatctatggtaacagcaatcggccctcaggagttcctgaccgattctctggatccaagtctggtacctcagcctccctggccatcactggactccaggctgaggatgaggctgattattactgccagtcctatgacagcagcctgtccggacatgtggtattcggtggaggaaccaagctgaccgtccta

SEQ ID NO: 94 - DNA, 759 bp, Artificial sequence
  Synthetic polynucleotide
  misc_feature: encodes the peptide sequence of RYRgerm_102919_33
gaggtgcagctggttgagtcgggaggaggtttggtacagcctggaggttccctgagactctcctgtgcagcctctggattcaccttctcgagctatagcatgaactgggtccgccaggctccaggtaagggactggagtgggtttcatacatttctagttcaagtagtaccatatactacgcagactctgtgaagggacgattcaccatctccagagacaatgccaagaactcactgtatctgcaaatgaacagcctgagagccgaggacacggctgtgtattactgtgcgaggtatcgatttccggatttttggagtggttaccctaactacggtatggacgtctggggacaaggaacaacagtcacagtttcatcaggtggaggaggttcaggtggaggtggctctggaggtggtggatctcagtctgttttgacgcagccgccctcagtgtctggtgccccagggcagagggtcaccatctcctgcactggaagcagctccaacatcggtgcaggttatgatgtacactggtaccagcagcttccaggaacagcccccaaactcctcatctatggtaacagcaatcggccctcaggagttcctgaccgattctctggatccaagtctggtacctcggcctccctggccatcactggactccaggctgaggatgaggctgattattactgccagtcctatgacagcagcctgtccggacatgtggtattcggtggaggaaccaagctgaccgtccta

SEQ ID NO: 95 - protein, 110 aa, Artificial sequence
  Synthetic polypeptide
  misc_feature (26)..(34): LCDR1
  misc_feature (52)..(54): LCDR2
  misc_feature (91)..(100): LCDR3
QSALTQPPSASGSPGQSVTISCTGTSSDVGGYNYVSWYQRHPGKAPKLMIYEVNKRPSGVPDRFSGSKSGDTASLTVSGLQAEDEADYYCSSYAGSPYVVFGGGTQLTVL

SEQ ID NO: 96 - protein, 126 aa, Artificial sequence
  Synthetic polypeptide
QVQLQESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPDFWSGYPNYGMDVWGQGTTVTVSS

SEQ ID NO: 97 - protein, 107 aa, Artificial sequence
  Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGHVVFGGGT

SEQ ID NO: 98 - protein, 8 aa, Artificial sequence
  Synthetic polypeptide
GFTLSDYS

SEQ ID NO: 99 - protein, 8 aa, Artificial sequence
  Synthetic polypeptide
ISSRSSYI

SEQ ID NO: 100 - protein, 14 aa, Artificial sequence
  Synthetic polypeptide
TGGQGYSGYDGFQH

SEQ ID NO: 101 - protein, 8 aa, Artificial sequence
  Synthetic polypeptide
SSNIGSNS

SEQ ID NO: 102 - protein, 3 aa, Artificial sequence
  Synthetic polypeptide
  misc_feature: CDR2 OF 3F1 VL
SNT

SEQ ID NO: 103 - protein, 11 aa, Artificial sequence
  Synthetic polypeptide
ASWDDSLNGGV

SEQ ID NO: 104 - protein, 13 aa, Artificial sequence
  Synthetic polypeptide
AASGFTFSSYSMN

SEQ ID NO: 105 - protein, 10 aa, Artificial sequence
  Synthetic polypeptide
YISSSSSTIY

SEQ ID NO: 106 - protein, 19 aa, Artificial sequence
  Synthetic polypeptide
ARYRLPDFWSGYPNYGMDV

SEQ ID NO: 107 - protein, 14 aa, Artificial sequence
  Synthetic polypeptide
TGSSSNIGAGYDVH

SEQ ID NO: 108 - protein, 8 aa, Artificial sequence
  Synthetic polypeptide
YGNSNRPS

SEQ ID NO: 109 - protein, 12 aa, Artificial sequence
  Synthetic polypeptide
QSYDSSLSGHVV

SEQ ID NO: 110 - protein, 19 aa, Artificial sequence
  Synthetic polypeptide
ARYRLPNFWSGYPNYGTDV